(19) United States
(12) Patent Application Publication
(10) Pub. No.: US 2015/0254421 A1
(43) Pub. Date: Sep. 10, 2015

(54) METHODS OF DIAGNOSING AMYLOID PATHOLOGIES USING ANALYSIS OF AMYLOID-BETA ENRICHMENT KINETICS

(71) Applicant: Washington University, St. Louis, MO (US)

(72) Inventors: Randall Bateman, St. Louis, MO (US); Bruce W. Patterson, St. Louis, MO (US); Donald L. Elbert, St. Louis, MO (US)

(21) Appl. No.: 14/717,453

(22) Filed: May 20, 2015

Related U.S. Application Data
(63) Continuation-in-part of application No. PCT/US2013/071042, filed on Nov. 20, 2013.
(60) Provisional application No. 61/728,692, filed on Nov. 20, 2012.

(51) Int. Cl. G06F 19/00; A61B 5/00

(52) U.S. Cl. CPC G06F 19/3437; A61B 5/4088

(57) ABSTRACT

A method of diagnosing an amyloid pathology in the central nervous system of a patient using measurements of enrichment kinetics of at least one amyloid-β isoform is provided. In addition, a model to predict enrichment kinetics of at least one amyloid-β isoform, methods of calibrating the model, and methods of using the model to diagnosing an amyloid pathology in the central nervous system of a patient are provided.

### REFERENCE TO RELATED APPLICATIONS

This application claims priority to PCT Patent Application PCT/US2013/071042 filed on Nov. 20, 2013, which claims priority to U.S. Provisional Patent Application No. 61/728,692 filed on Nov. 20, 2012, and entitled “METHODS OF DIAGNOSING AMYLOID PATHOLOGIES USING ANALYSIS OF AMYLOID-BETA ENRICHMENT KINETICS”, each of which is hereby incorporated herein by reference in its entirety.

### GOVERNMENTAL RIGHTS IN THE INVENTION

This invention was made with government support under 5P01AG026276-S1 awarded by the National Institute on Aging, and R-01-NS065667 awarded by the National Institutes of Health. The government has certain rights in the invention.

### FIELD OF THE INVENTION

This disclosure generally relates to methods of diagnosing an amyloid pathology in the central nervous system of a patient using measurements of enrichment kinetics of at least one amyloid-β isoform. In addition, this disclosure relates to methods of developing and using a mathematical model to predict enrichment kinetics of at least one amyloid-β isoform and to diagnose an amyloid pathology in the central nervous system of a patient using the model.

### REFERENCE TO SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821(f).

### BACKGROUND OF THE INVENTION

Alzheimer's Disease (AD) is the most common cause of dementia and is an increasing public health problem. AD, like other central nervous system (CNS) degenerative diseases, is characterized by disturbances in protein production, accumulation, and clearance. In AD, dysregulation in the metabolism of the protein, amyloid-beta (Aβ), is indicated by a massive buildup of this protein in the brains of those with the disease. Because of the severity and increasing prevalence of this disease in the population, it is urgent that better treatments be developed.

The pathogenic causes of Alzheimer's disease are not fully understood, partly due to the difficulty in demonstrating the steps that lead to dementia in humans. Although rare, autosomal dominant AD (ADAD) can be predicted with near 100% certainty in individuals with specific mutations in presenilin 1 (PSEN1), presenilin 2 (PSEN2), or the amyloid precursor protein (APP). Recent findings suggest that a series of ADAD pathophysiological changes occur in the brain decades before clinical dementia manifests. However, the mechanisms by which these mutations lead to AD pathophysiology are not well understood.

The amyloid hypothesis predicts that AD is caused by increased production or decreased clearance of Aβ in the brain, resulting in amyloidosis (the deposition of amyloid proteins in an organ or tissue) and AD's pathologic hallmark of amyloid plaques, which are principally composed of Aβ42. An APP mutation which reduces Aβ production is associated with a strong protective effect against AD, while duplication of APP or mutations which are thought to increase Aβ or Aβ42 cause dominantly inherited AD. Aβ is cleaved from the c-terminal fragment of APP (C99) by PSEN1 and PSEN2, the enzymatic components of gamma-secretase. In cell culture and in plasma, PSEN mutations have been associated with increased Aβ42:Aβ40 ratio, which is hypothesized to increase the risk of amyloidosis. However, others have found that neither the Aβ42:Aβ40 ratio nor Aβ42 levels are increased in vitro. Further, findings of paradoxically reduced cerebrospinal fluid (CSF) Aβ42 concentrations in ADAD patients do not appear to directly support the predicted increased Aβ42 production as an etiologic mechanism in dominantly inherited AD.

Sporadic AD may be characterized by decreased Aβ clearance measured by stable isotope labeling kinetics (SILK). Both sporadic AD and ADAD are associated with lower CSF Aβ42 concentrations and Aβ42:Aβ40 ratios. However, PSEN ADAD mutations are hypothesized to cause increased Aβ42 production, although direct evidence for increased production of Aβ42 in humans has not been reported.

A need exists, therefore, for a method for modeling the in vivo kinetics of Aβ. In particular, a method is needed for modeling the in vivo fractional synthesis rate and clearance rate of proteins associated with a neurodegenerative disease, e.g., the metabolism of Aβ in AD. Such a model may serve as a useful tool in research directed to the characterization and treatment of the underlying processes of AD.

### SUMMARY OF THE INVENTION

The present disclosure generally relates to systems and methods of modeling and calibrating models for the metabolism and trafficking of CNS biomolecules in a patient.

In one aspect, a method of calibrating a compartmental model for the steady-state kinetics of a biomolecule includes obtaining data values for a level of a labeled moiety in a patient as a function of time. A fraction of the biomolecule is the labeled moiety. The method includes modeling a metabolic pathway of the biomolecule with a compartmental model based on the obtained data values for the labeled moiety, plotting a result of the compartmental model, and comparing a plot of the result to another plot of measured data values. If the plot of the model results matches the other plot of measured data values then the model is sufficiently calibrated. Conversely, if the plot of the result does not match the other plot of the measured data values, then at least one rate constant of the compartmental model is modified. The metabolic pathway of the biomolecule is remodeled using the at least one modified rate constant. The remodeled result of the compartmental model is plotted and compared to the other plot of the measured data values. The actions of comparing and modifying at least one rate constant are repeated, as necessary, to produce a plot the matches the plot of measured data values.

In various other aspects, the method may be performed on one or more computing devices. The computing devices may be distributed across a network or stand-alone devices. In one aspect, the computing device may be used to permit a user to modify and compare plots simultaneously, and in near real time.

In a second aspect, a method for detecting amyloid pathology in the central nervous system of a patient provided that includes: i) determining one or more kinetic parameters of Aβ42 and at least one other Aβ peptide, (ii) comparing the Aβ42 kinetic parameter and the same kinetic parameter for a second Aβ measurement, and (iii) determining whether a subject has amyloid pathology based on a difference between the two kinetic parameters. The kinetic parameter may be selected from the group consisting of fractional synthesis rate, peak time, peak enrichment, initial downturn monoexponential slope, terminal monoexponential slope, and a combination thereof. Two or more kinetic parameters may be determined, three or more kinetic parameters may determined, four or more kinetic parameters may be determined, or at least five kinetic parameters may be determined. The kinetic parameter may be fractional synthesis rate and the Aβ42 fractional synthesis rate may be faster than the fractional synthesis rate for the second Aβ measurement, the kinetic parameter may be peak time and the Aβ42 peak time may be earlier than the peak time for the second Aβ measurement, the kinetic parameter may be peak enrichment and the Aβ42 peak enrichment may be lower than the peak enrichment for the second Aβ measurement, the kinetic parameter may be initial downturn monoexponential slope and the initial Aβ42 slope may be faster than the initial slope for the second Aβ measurement, or the kinetic parameter may be terminal monoexponential slope and the terminal Aβ42 slope may be slower than the terminal slope for the second Aβ measurement. The one or more kinetic parameters may be determined by stable isotope labeling kinetics. A labeled amino acid may be administered to the subject hourly for a time period selected from the group consisting of 6 to 12 hours, 6 to 9 hours, and 9 to 12 hours. The amount of labeled peptide and the amount of unlabeled peptide may be detected by a means selected from the group consisting of mass spectrometry, tandem mass spectrometry, and a combination thereof. The one or more kinetic parameters may be determined using a mathematical model for the enrichment kinetics of Aβ. The method may further include calculating the isotopic enrichment of Aβ42 compared to the second Aβ measurement at a single timepoint after administration of the labeled amino acid to the patient. The second Aβ measurement may be selected from the group consisting of an Aβ peptide other than Aβ42 and total Aβ. The Aβ peptide other than Aβ42 may be Aβ38 or Aβ40. The method may further include (i) calculating the ratio between the Aβ42 kinetic parameter and the same kinetic parameter for the second Aβ measurement, and (ii), comparing the ratio calculated in (i) to a threshold value, wherein a value lower than the threshold indicates the patient has amyloid plaques.

In a third aspect, a method to diagnose an amyloid pathology in a patient is provided. The method includes (i) creating a mathematical model for the steady-state kinetics of Aβ including a set of model parameters (ii) calculating ten times kₑₓ₄₂ and adding that to the FTR ratio, and (iii) comparing the value from (ii) to a threshold value, wherein a value lower than the threshold value indicates a subject has Alzheimer's Disease. The set of model parameters includes: kₑₓ₄₂, a rate constant for an irreversible loss for Aβ42, and a rate constant for an irreversible loss for Aβ40. kₑₓ₄₂ describes the rate of entry of Aβ42 into the exchange compartment and the FTR ratio is the ratio of the rate constants for irreversible loss for Aβ42 versus Aβ40. The amyloid pathology may be selected from the group consisting of amyloid plaques, altered Aβ kinetics (such as Aβ amyloidosis), and Alzheimer's Disease.

In a fourth aspect, a method of calibrating a model to estimate a time course of enrichment kinetics of at least one Aβ isoform is provided. The method includes: a) obtaining data values for an amount of a labeled moiety introduced into a patient as a function of time, wherein a fraction of the at least one Aβ isoforms includes the labeled moiety; b) modeling a metabolic pathway of the at least one Aβ isoform with the model based on the obtained data values to calculate a set of model parameters and an estimated time course of enrichment kinetics of the at least one amyloid; and c) comparing the estimated time course of enrichment kinetics of the at least one Aβ isoform to a measured time course of enrichment kinetics of the at least one Aβ isoform obtained from the patient. If the estimated time course of enrichment kinetics matches the measured time course of enrichment kinetics, the model determines that the compartmental model is calibrated. If the estimated time course of enrichment kinetics does not match the measured time course of enrichment kinetics the model may modify at least one of the set of model parameters and remodel metabolic pathway of the Aβ peptide using the modified model parameters to calculate a new estimated time course of enrichment of the at least one amyloid; these steps may be repeated until the compartmental model is calibrated.

In a fifth aspect, an amyloid kinetics modeling system for estimating a time course of enrichment kinetics of at least one Aβ isoform is provided. The system may include: a) at least one processor; and b) a CRM containing an amyloid kinetics application including a plurality of modules executable on the at least one processor. The plurality of modules may include: i) a plasma module to represent infusion of a labeled moiety into the plasma of a patient and to represent transport of the labeled moiety across the blood brain barrier (BBB) of the patient; ii) a brain tissue module to represent incorporation of the labeled moiety into APP and formation of C99; iii) an amyloid kinetics module to represent cleavage of the C99 to form at least one Aβ isoform and subsequent kinetics of the at least one Aβ isoform within the brain of the patient; iv) a CSF module to represent transport of the at least one Aβ isoform into the CSF of the patient; v) a model tuning module to iteratively adjust a set of model parameters defining a dynamic response of the model to an input time history of plasma leucine enrichment into the plasma module in order to optimize a match between predicted enrichment kinetics and measured enrichment kinetics of the at least one Aβ isoform in the patient; and vi) a GUI module to generate one or more forms used to receive inputs to the system and to deliver output from the system. The plasma module includes a plasma amino acid compartment including a plasma concentration of at least one amino acid, wherein the plasma concentration of the at least one amino acid may be determined using an input including a time history of an infusion of a labeled amino acid into a patient. The brain tissue module includes: a) an APP compartment including a total amount of APP; b) an APP incorporation rate including a rate of incorporation of the at least one amino acid from the plasma amino acid compartment into an APP molecule in the APP compartment; c) a C99 compartment including a total amount of C99 c-terminal fragments; d) a C99 formation rate including a rate of formation of the C99 c-terminal fragments in the C99 compartment from the APP molecules; and e) a C99 clearance rate including a rate of disappearance of the C99 c-terminal fragments from the C99 compartment. The amyloid kinetics module includes: a) a soluble Aβ42 isoform compartment including an amount of a soluble Aβ42 isoform; b) an Aβ42 isoform formation rate including a rate of formation of soluble Aβ42 isoform from the C99 c-terminal fragments; c) an Aβ42 isoform clearance rate including a rate of disappearance of Aβ42 isoforms from the soluble Aβ compartment; d) an Aβ42 incorporation rate including a rate of transformation of the soluble Aβ42 isoform to an incorporated Aβ42 isoform; and e) a recycled Aβ42 compartment including a total amount of incorporated Aβ42 isoform. The CSF module includes a) a CSF Aβ42 compartment including a total amount of CSF Aβ42 isoforms; b) a CSF Aβ42 transfer rate including a rate of transfer of soluble Aβ42 isoform from the soluble Aβ42 compartment to the CSF Aβ42 compartment; and c) a CSF Aβ42 clearance rate including a rate of disappearance of CSF Aβ42 from the CSF Aβ42 pool. The amyloid kinetics module may further include: a) a soluble comparison Aβ isoform compartment including an amount of a soluble comparison Aβ isoform; b) a comparison Aβ isoform formation rate including a rate of formation of soluble comparison Aβ isoform from the C99 c-terminal fragments; and c) a comparison Aβ isoform clearance rate including a rate of disappearance of soluble comparison Aβ isoforms from the soluble comparison Aβ isoform compartment. The CSF module may further include: a) a CSF comparison Aβ isoform compartment including a total amount of CSF comparison Aβ isoforms; b) a CSF comparison Aβ isoform transfer rate including a rate of transfer of soluble comparison Aβ isoform from the soluble comparison Aβ isoform compartment to the CSF comparison Aβ isoform compartment; and c) a CSF comparison Aβ isoform clearance rate including a rate of disappearance of CSF comparison Aβ isoform from the CSF comparison Aβ isoform compartment. The comparison Aβ isoform may be chosen from Aβ38 and Aβ40.

In a sixth aspect, a system for estimating the kinetics of amyloid-beta (Aβ) in the CNS of a patient is disclosed that includes: at least one processor; and a CNS Aβ kinetic model application including a plurality of modules executable using the at least one processor. The modules may include: a) a plasma amino acid module to estimate a plasma amino acid compartment including a plasma concentration of at least one amino acid; b) an APP incorporation module to estimate an APP incorporation rate including a rate of incorporation of the at least one amino acid from the plasma amino acid compartment into an APP molecule in an APP compartment; c) an APP module to estimate the APP compartment including a total amount of APP molecules; d) a C99 formation module to estimate a C99 formation rate including a rate of formation of a C99 c-terminal fragment in a C99 compartment from the APP molecules; e) a C99 clearance module to estimate a C99 clearance rate including a rate of disappearance of the C99 c-terminal fragment from the C99 compartment; e) a C99 module to estimate the C99 compartment including a total amount of the C99 c-terminal fragments; f) a free Aβ formation module to estimate at least one free Aβ isoform formation rate, each free Aβ isoform formation rate including a rate of formation of a free Aβ isoform in a free Aβ compartment from the C99 c-terminal fragments; g) a free Aβ clearance module to estimate at least one free Aβ isoform clearance rate, each free Aβ isoform clearance rate including a rate of disappearance of one of the free Aβ isoforms from the free Aβ compartment; h) a free Aβ module to estimate the free Aβ compartment including the total amount of all free Aβ isoforms; i) a free Aβ recycling module to estimate at least one free Aβ incorporation rate, each free Aβ incorporation rate including a rate of transformation of a free Aβ isoform to an incorporated Aβ isoform in a recycled Aβ compartment, and at least one Aβ recycling rate, each Aβ recycling rate including a rate of recycling an incorporated Aβ isoform in the recycled Aβ compartment back into a free Aβ isoform in the free Aβ compartment; j) a CSF Aβ transfer module to estimate at least one CSF Aβ transfer rate, each Aβ transfer rate including a rate of transfer of one free Aβ isoform from the free Aβ compartment to a CSF Aβ compartment; k) a CSF Aβ clearance module to estimate at least one CSF Aβ clearance rate, each CSF Aβ clearance rate including a rate of disappearance of one CSF Aβ isoform from the CSF Aβ compartment; and I) a CSF Aβ module to estimate the CSF Aβ compartment including the total amount of CSF Aβ isoforms. The Aβ isoforms may be chosen from Aβ38, Aβ40, and Aβ42. At least a portion of the plasma amino acid compartment may include a plasma concentration of at least one labeled amino acid. At least a portion of the APP compartment may include an amount of enriched APP molecules incorporating the at least one labeled amino acid. At least a portion of the C99 compartment may further include an amount of enriched C99 c-terminal fragments formed from the amount of enriched APP molecules. At least a portion of the Aβ isoforms may further include an amount of enriched Aβ isoforms formed from the amount of enriched C99 c-terminal fragments. The CSF Aβ transfer module may further estimate at least one CSF Aβ delay, each CSF Aβ delay including a delay in the transfer of one free Aβ isoform from the free Aβ compartment to the CSF Aβ compartment. The at least one CSF Aβ transfer rate may be represented by a fluid flow of ISF within the brain.

In a seventh aspect, a method of using a model of amyloid β (Aβ) isoform enrichment kinetics is provided that includes: a) obtaining from a patient measured Aβ enrichment kinetics data including a time course of concentration of a labeled moiety infused into the patient, a measured time course of Aβ42 enrichment kinetics in the CSF of the patient, and a measured time course of at least one other comparison Aβ isoform enrichment kinetics in the patient; b) inputting the measured Aβ enrichment kinetics data into the model, wherein the model represents enrichment kinetics of Aβ42 and the at least one other comparison Aβ isoform; c) obtaining a set of model parameters from the model; d) calculating a model index including a mathematical combination of at least two model parameters from the model; e) comparing the model index to a pre-selected threshold range; and f) identifying a disease state of the patient if the model index falls outside of the threshold range. The disease state may be identified as Alzheimer's if the model index falls outside of the threshold range. The severity of the disease state may be identified by comparing the model index to a pre-selected correlation of the disease state with the model index. The correlation of the disease state may be a correlation of the model index with PIB imaging values obtained from a population of patients with a range of disease states. The measured Aβ enrichment kinetics data from a patient may be obtained by the SILK method. The labeled moiety may be labeled leucine. The at least one other comparison Aβ isoform may be chosen from Aβ38 and Aβ40. The model parameters may be chosen from: concentration of Aβ isoforms, rates of transfer, rates of irreversible loss, rates of exchange, rates of delay, and combinations thereof. The model index may be calculated using a rate of irreversible loss of Aβ42 and a rate of transfer of Aβ42. The model parameters may be obtained by iteratively varying the model parameters until a best fit of the estimated Aβ enrichment kinetics to the measured Aβ enrichment kinetics is obtained.

In an eighth aspect, an amyloid kinetics modeling system for estimating a time course of enrichment kinetics of at least one Aβ isoform is provided that includes: a) at least one processor; and b) a CRM containing an amyloid kinetics application including a plurality of modules executable on the at least one processor. The plurality of modules may include: i) a plasma module to represent infusion of a labeled moiety into the plasma of a patient and to represent transport of the labeled moiety across the blood brain barrier (BBB) of the patient; ii) a brain tissue module to represent incorporation of the labeled moiety into APP and formation of C99; iii) an amyloid kinetics module to represent cleavage of the C99 to form at least one Aβ isoform and subsequent kinetics of the at least one Aβ isoform within the brain of the patient; iv) a CSF module to represent transport of the at least one Aβ isoform into the CSF of the patient; v) a blood enrichment module to represent transport of the at least one Aβ isoform into the blood of the patient; v) a model tuning module to iteratively adjust a set of model parameters defining a dynamic response of the model to an input time history of plasma leucine enrichment into the plasma module in order to optimize a match between predicted enrichment kinetics and measured enrichment kinetics of the at least one Aβ isoform in the patient; and vi) a GUI module to generate one or more forms used to receive inputs to the system and to deliver output from the system. The plasma module includes a plasma amino acid compartment including a plasma concentration of at least one amino acid, wherein the plasma concentration of the at least one amino acid may be determined using an input including a time history of an infusion of a labeled amino acid into a patient. The brain tissue module includes: a) an APP compartment including a total amount of APP; b) an APP incorporation rate including a rate of incorporation of the at least one amino acid from the plasma amino acid compartment into an APP molecule in the APP compartment; c) a C99 compartment including a total amount of C99 c-terminal fragments; d) a C99 formation rate including a rate of formation of the C99 c-terminal fragments in the C99 compartment from the APP molecules; and e) a C99 clearance rate including a rate of disappearance of the C99 c-terminal fragments from the C99 compartment. The amyloid kinetics module includes: a) a soluble Aβ42 isoform compartment including an amount of a soluble Aβ42 isoform; b) an Aβ42 isoform formation rate including a rate of formation of soluble Aβ42 isoform from the C99 c-terminal fragments; c) an Aβ42 isoform clearance rate including a rate of disappearance of Aβ42 isoforms from the soluble Aβ compartment; d) an Aβ42 incorporation rate including a rate of transformation of the soluble Aβ42 isoform to an incorporated Aβ42 isoform; and e) a recycled Aβ42 compartment including a total amount of incorporated Aβ42 isoform. The CSF module includes: a) a CSF Aβ42 compartment including a total amount of CSF Aβ42 isoforms; b) a CSF Aβ42 transfer rate including a rate of transfer of soluble Aβ42 isoform from the soluble Aβ42 compartment to the CSF Aβ42 compartment; and c) a CSF Aβ42 clearance rate including a rate of disappearance of CSF Aβ42 from the CSF Aβ42 pool. The amyloid kinetics module may further include: a) a soluble comparison Aβ isoform compartment including an amount of a soluble comparison Aβ isoform; b) a comparison Aβ isoform formation rate including a rate of formation of soluble comparison Aβ isoform from the C99 c-terminal fragments; and c) a comparison Aβ isoform clearance rate including a rate of disappearance of soluble comparison Aβ isoforms from the soluble comparison Aβ isoform compartment. The CSF module may further include: a) a CSF comparison Aβ isoform compartment including a total amount of CSF comparison Aβ isoforms; b) a CSF comparison Aβ isoform transfer rate including a rate of transfer of soluble comparison Aβ isoform from the soluble comparison Aβ isoform compartment to the CSF comparison Aβ isoform compartment; and c) a CSF comparison Aβ isoform clearance rate including a rate of disappearance of CSF comparison Aβ isoform from the CSF comparison Aβ isoform compartment. The blood enrichment module includes: a) a blood Aβ42 compartment including a total amount of blood Aβ42 isoforms; b) a blood Aβ42 transfer rate including a rate of transfer of soluble Aβ42 isoform from the soluble Aβ42 compartment to the blood Aβ42 compartment; and c) a blood Aβ42 clearance rate including a rate of disappearance of blood Aβ42 from the blood Aβ42 pool. The comparison Aβ isoform may be chosen from Aβ38 and Aβ40.

In a ninth aspect, an amyloid kinetics modeling system for estimating a time course of enrichment kinetics of at least one Aβ isoform is provided that may include: a) at least one processor; and b) a CRM containing an amyloid kinetics application including a plurality of modules executable on the at least one processor. The plurality of modules may include: i) a plasma module to represent infusion of a labeled moiety into the plasma of a patient and to represent transport of the labeled moiety across the blood brain barrier (BBB) of the patient; ii) a brain tissue module to represent incorporation of the labeled moiety into APP and formation of C99; iii) an amyloid kinetics module to represent cleavage of the C99 to form at least one Aβ isoform and subsequent kinetics of the at least one Aβ isoform within the brain of the patient; v) a blood enrichment module to represent transport of the at least one Aβ isoform into the blood of the patient; v) a model tuning module to iteratively adjust a set of model parameters defining a dynamic response of the model to an input time history of plasma leucine enrichment into the plasma module in order to optimize a match between predicted enrichment kinetics and measured enrichment kinetics of the at least one Aβ isoform in the patient; and vi) a GUI module to generate one or more forms used to receive inputs to the system and to deliver output from the system. The plasma module includes a plasma amino acid compartment including a plasma concentration of at least one amino acid, wherein the plasma concentration of the at least one amino acid may be determined using an input including a time history of an infusion of a labeled amino acid into a patient. The brain tissue module includes: a) an APP compartment including a total amount of APP; b) an APP incorporation rate including a rate of incorporation of the at least one amino acid from the plasma amino acid compartment into an APP molecule in the APP compartment; c) a C99 compartment including a total amount of C99 c-terminal fragments; d) a C99 formation rate including a rate of formation of the C99 c-terminal fragments in the C99 compartment from the APP molecules; and e) a C99 clearance rate including a rate of disappearance of the C99 c-terminal fragments from the C99 compartment. The amyloid kinetics module includes: a) a soluble Aβ42 isoform compartment including an amount of a soluble Aβ42 isoform; b) an Aβ42 isoform formation rate including a rate of formation of soluble Aβ42 isoform from the C99 c-terminal fragments; c) an Aβ42 isoform clearance rate including a rate of disappearance of Aβ42 isoforms from the soluble Aβ compartment; d) an Aβ42 incorporation rate including a rate of transformation of the soluble Aβ42 isoform to an incorporated Aβ42 isoform; and e) a recycled Aβ42 compartment including a total amount of incorporated Aβ42 isoform. The amyloid kinetics module may further include: a) a soluble comparison Aβ isoform compartment including an amount of a soluble comparison Aβ isoform; b) a comparison Aβ isoform formation rate including a rate of formation of soluble comparison Aβ isoform from the C99 c-terminal fragments; and c) a comparison Aβ isoform clearance rate including a rate of disappearance of soluble comparison Aβ isoforms from the soluble comparison Aβ isoform compartment. The blood enrichment module includes: a) a blood Aβ42 compartment including a total amount of blood Aβ42 isoforms; b) a blood Aβ42 transfer rate including a rate of transfer of soluble Aβ42 isoform from the soluble Aβ42 compartment to the blood Aβ42 compartment; and c) a blood Aβ42 clearance rate including a rate of disappearance of blood Aβ42 from the blood Aβ42 pool. The comparison Aβ isoform may be chosen from Aβ38 and Aβ40.

### BRIEF DESCRIPTION OF THE DRAWINGS

The application file contains at least one photograph executed in color. Copies of this patent application publication with color photographs will be provided by the Office upon request and payment of the necessary fee.

FIG. 1 is a schematic diagram illustrating the processing of amyloid precursor protein (APP) [SEQ. ID. NO. 1] into amyloid-β (Aβ) within a cell.

FIG. 2 is a schematic diagram illustrating the processing of Aβ and paths the Aβ isoforms may take in vivo.

FIG. 3 is a simplified diagram illustrating the overall architecture of a compartment model for the metabolism and trafficking of Aβ.

FIG. 4 is a detailed diagram illustrating the detailed architecture of a compartment model for the metabolism and trafficking of Aβ with measured Aβ concentrations at the CSF.

FIG. 5 is a graph summarizing a time course of plasma leucine enrichment normalized to the enrichment plateau during and after labeled leucine infusion.

FIGS. 6A-6B illustrate an average Aβ isotropic kinetic time course profile in CSF of non-mutation carriers as an isotropic enrichment ratio (FIG. 6A) and as enrichments normalized to plasma leucine plateau enrichments with a model fit line (FIG. 6B). FIGS. 6C-6D illustrate an average Aβ isotropic kinetic time course profile in CSF of PIB− mutation carriers as an isotropic enrichment ratio (FIG. 6C) and as enrichments normalized to plasma leucine plateau enrichments with a model fit line (FIG. 6D). FIGS. 6E-6F illustrate an average Aβ isotropic kinetic time course profile in CSF of PIB+ mutation carriers as an isotropic enrichment ratio (FIG. 6E) and as enrichments normalized to plasma leucine plateau enrichments with a model fit line (FIG. 6F).

FIG. 7 is a block diagram illustrating a computing environment for calibrating and executing a compartment model according to one embodiment.

FIG. 8 is a block diagram illustrating a computing device for calibrating and executing a compartment model according to one embodiment.

FIG. 9 is a block diagram illustrating a data source that may be used when calibrating and executing a compartment model according to one embodiment.

FIG. 10 is a block diagram illustrating a computing device for calibrating and executing a compartment model according to one embodiment.

FIG. 11 is a flowchart illustrating one method of calibrating a compartmental model according to one embodiment.

FIG. 12 is a diagram illustrating a modified architecture of a compartment model for the metabolism and trafficking of Aβ in one aspect.

FIG. 13 is a diagram illustrating flow of Aβ42 from the ventricles to the brain surface/CSF.

FIGS. 14A-14B are graphs summarizing the pressure (FIG. 14A) and velocity of flow (FIG. 14B) from the ventricles to the brain surface/CSF.

FIG. 15 is a flowchart illustrating a method of using the kinetic model to identify a patient's disease state.

FIG. 16 is a block diagram illustrating the modules of an amyloid kinetics modeling system in an aspect.

FIG. 17 is a schematic diagram illustrating the nodes of a flow model in one aspect.

FIG. 18 is a schematic diagram illustrating a detailed architecture of a single node of a flow model in one aspect.

FIG. 19A-B depict two graphs showing a monoexponential slope fit to the descending enrichment on the back end of the kinetic tracer curve for Aβ42. FIG. 19A illustrates that the entire back end of the peak is monoexponential to the end of the time course (36 h). In contrast, FIG. 19B illustrates that there is evidence of a 2nd, slower exponential tail to the peak; in these cases, an initial rapid slope that visually excludes the slower tail is selected. The graphs show the natural log of enrichment vs. time; the monoexponential slope FCR is the negative of the slope.

FIG. 20A-C depicts three graphs showing that a comparison of isotopic enrichments around the midpoint on the back end of the kinetic tracer curve is able to discriminate the PIB groups highly significantly. FIG. 20A shows the ratio of Aβ42 percent labeled/Aβ40 percent labeled at 23 hours graphed on the y-axis and PIB staining graphed on the x-axis. A threshold ratio of 0.9 is indicated by the dashed line. FIG. 20B shows the average of the ratio of Aβ42 percent labeled/Aβ40 percent labeled at 23 hours and 24 hours graphed on the y-axis and PIB staining graphed on the x-axis. A threshold ratio of 0.9 is indicated by the dashed line. FIG. 20C shows the calculated values of ten times kₑₓ₄₂ added to ratio of the rate constants for irreversible loss for Aβ42 versus Aβ40 (10×kₑₓ₄₂ FTR ratio) plotted as a function of PIB staining. A threshold ratio of 1.75 is indicated by the dashed line. MC+=patients with PSEN1 or PSEN2 mutations that were PIB positive by PET; MC−=patients with PSEN1 or PSEN2 mutations that were PIB negative by PET; NC=non-carrier mutation carrier sibling controls.

FIG. 21 is a detailed diagram illustrating the detailed architecture of a compartment model for the metabolism and trafficking of Aβ with measured Aβ concentrations at the blood.

FIG. 22 is of a block diagram illustrating a machine in the example form of a computer system 500 within which instructions 506 for causing the machine to perform any one or more of the methodologies discussed herein may be executed by one or more hardware processors 502.

FIG. 23 provides Eqn. (11.3.7).

FIG. 24 provides Eqn. (11.3.8).

FIG. 25A-B depicts two graphs showing the morphology of Aβ isotopic labeling curves. Isotopically labeled leucine was infused into human volunteers for 9 hours, while cerebrospinal fluid (CSF) was collected via lumbar puncture hourly for 36 hours. The tracer-to-tracee ratio (TTR) of Aβ peptides was measured by mass spectrometry and converted to fractional labeling of Aβ peptides. This was then normalized by the mean fractional labeling of leucine in blood plasma during the infusion period. FIG. 25A depicts an Aβ labeling curve for presenilin-1 mutation carriers with amyloid plaques validated by PET PIB. Note that the Aβ42 isotopic labeling curve is markedly different from those of Aβ38 and Aβ40. FIG. 25B depicts an Aβ labeling curve for non-mutation carriers with without amyloid plaques. Note that the isotopic labeling curves are similar for Aβ42, Aβ40, and Aβ38.

FIG. 26A-D depicts four graphs showing predicted time course for Aβ precursors. The predicted time course of plasma leucine, APP, C99 and Aβ42 in the brain compartment are shown in FIG. 26A and FIG. 26C. The predicted time course of Aβ42 in the brain, first delay compartment, second delay compartment, and third delay compartment are shown in FIG. 26B and FIG. 26D. The third delay compartment corresponds to the lumber sub-arachnoid space from which CSF was sampled. The rate equations were solved numerically with re-optimized parameters listed in Appendix H for a presenilin-1 mutation carrier with plaques validated by PET PIB in FIG. 26A and FIG. 26B, and a non-carrier without plaques in FIG. 26C and FIG. 26D.

FIG. 27A-F depicts two drawings and four graphs showing fractional synthesis rate (FSR) and fractional clearance rate (FCR) for compartmental models with multiple pathways. In FIG. 27A, a simple model of a single precursor with constant labeling fraction during the labeling phase, which produces two products. In FIG. 27B, the labeling kinetics of each product show that variation of the production rate constants (k₁ and k₂) have no effect on labeling kinetics. Variation of the clearance rate constants (v₁ and v₂) has the only impact on labeling kinetics, and FSR and FCR are both provide good estimates of v₁ or v₂. In FIG. 27C, a two-step model in which precursor A is maintained at constant concentration during the labeling phase, but produces a precursor B, which then produces two products. In FIG. 27D, with v₁ and v₂ given the same value, the values of k₁ and k₂ were set to different values, however, their sum remained constant. Regardless of the individual values of k₁ and k₂ the labeling curves for both products overlapped. FCR was close to but slightly lower than v₁ and v₂, while FSR was difficult to associate with any of the parameters. In FIG. 27E, the values of k₁ and k₂ were set to different values, however, their sum remained constant. The value of v₁ was set to twice that of v₂. Regardless of the individual values of k₁ and k₂ the labeling curves for each product overlapped. FCR was a close to but slightly lower than v₁ or v₂. FSR was 47% higher when the clearance rate constant was twice as large. In FIG. 27F, production rate constants k₁ and k₂ were set equal to each other, but their sum was varied while setting v₁ and v₂ equal to each other. Changes in k₁+k₂ led to distinct labeling curves. FCR approached the value of v₁ and v₂ when k₁+k₂ became larger, but was much lower than v₁ and v₂ when k₁+k₂ was lower. FSR increased by 28% when k₁+k₂ was doubled.

FIG. 28A-B depicts two graphs showing sensitivity analysis of exact solution to the compartmental model. The rate equations corresponding to the compartmental model were solved analytically for the labeled fraction of Aβ42 in the third delay compartment (p_{Ab42d3L}), which corresponds to the fraction of labeled Aβ42 found in the lumbar CSF. The derivative of this function with respect to the listed parameters was taken and plotted as a function of time. Also plotted are the measured (‘Meas p’) and predicted (‘Model p’) fractional labeling, multiplied by 6 for readability. FIG. 28A depicts data from a presenilin-1 mutation carrier with plaques validated by PET PIB scans. FIG. 28B depicts data from a non-carrier without plaques.

FIG. 29A-B depicts two graphs showing changes in model predictions with changes in parameters. The indicated parameter values were increased by 0.1 h⁻¹. The rate equations were solved numerically with all other rate constants at their original values. FIG. 29A depicts data from a presenilin-1 mutation carrier with plaques validated by PET PIB scans. FIG. 29B depicts data from a non-carrier without plaques. The observed trends help to visualize the results of the sensitivity analysis shown in FIG. 28.

FIG. 30A-D depicts four graphs showing sensitivity analysis of time derivative of exact solution. The time derivative of the labeling time course between 5 and 14 h has previously been used to estimate production rate constants of kinetic systems (reference [7]). In FIG. 30A and FIG. 30B, ‘slope’ of the labeling curve (dp_{Ab42d3L}/dt; multiplied by 10 for readability) shows that the data are not well-described by a straight line between 5 and 14 h. In FIG. 30C and FIG. 30D, the sensitivity of dp_{Ab42d3L}/dt with respect to changes in the various parameters was evaluated. Also plotted are the measured (‘Meas p’) and predicted (‘Model p’) fractional labeling. FIG. 30A and FIG. 30C depict data from a presenilin-1 mutation carrier with plaques validated by PET PIB scans. FIG. 30B and FIG. 30D depict data from a non-carrier without plaques.

FIG. 31A-C depict three graphs showing sensitivity analysis of the monoexponential FCR. The time derivative of the logarithm of the labeling time course was previously used to estimate ‘clearance’ kinetics between 24 and 36 h (reference [7]). In FIG. 31A, the time derivative of −ln(p_{Ab42d3L}) is the instantaneous ‘monoexponential slope’ or FCR is shown for each subject. This is relatively constant for the non-carrier between 24 and 36 h, but varies considerably for the mutation carrier. In FIG. 31B and FIG. 31C, the sensitivity of d(−ln(p_{Ab42d3L}))/dt to changes in parameter values was evaluated. Also plotted are the measured (‘Meas p’) and predicted (‘Model p’) fractional labeling, scaled by 4 for readability. FIG. 31B depicts data from a presenilin-1 mutation carrier with plaques validated by PET PIB scans. FIG. 31C depicts data from a non-carrier without plaques.

Corresponding reference characters and labels indicate corresponding elements among the views of the drawings. The headings used in the figures should not be interpreted to limit the scope of the claims.

### DETAILED DESCRIPTION

Provided herein are methods for modeling the in vivo kinetics and metabolism of a CNS biomolecule, in particular one or more amyloid-β (Aβ) isoforms. As used herein, the term “CNS biomolecule” refers to a biomolecule synthesized in the central nervous system (CNS). A skilled artisan will appreciate that while a biomolecule may be synthesized in the CNS, the biomolecule may be transported to other compartments of the body, such that the biomolecule may be detected in the CNS, peripheral nervous system, or outside the nervous system (e.g. in the blood). The kinetic model may be developed and/or calibrated utilizing measured data from patients including, but not limited to the blood and/or the cerebrospinal fluid (CSF) of the patients. Blood, as defined herein, may refer to whole blood, plasma, serum, and any other blood fraction known in the art. This disclosure further provides methods for developing a model by determining and predicting steady state metabolic kinetic parameters. In addition, this disclosure additionally provides methods for modeling in vivo metabolism of one or more Aβ isoforms to determine concentrations of the Aβ isoforms at various states, fractional turnover rates of the one or more Aβ isoforms, and production rates of the one or more Aβ isoforms. Also provided are methods for using the model to identify a patient's disease state and predict aspects of Aβ isoform enrichment kinetics and/or concentrations within a patient. In particular, this disclosure relates to methods of modeling Aβ turnover kinetics in a kinetic model. In an aspect, the kinetic model may be a steady state compartmental model, a flow model, or any combination thereof without limitation. In an aspect, the kinetic model may be used to model the metabolism of any CNS biomolecule.

The method of developing the model may include, but is not limited to, measuring a concentration of a labeled moiety introduced into a patient over a period of time. The labeled moiety may be incorporated into an Aβ precursor within the patient. The method may further include measuring concentrations in a biological sample of the Aβ isoforms incorporating the labeled moiety in the patient, and incorporating the measured data into known or hypothesized relationships and/or metabolic processes. In an aspect, the model may predict the measured values. The model may be developed by calibrating the predicted values against measured values and adjusting a set of model parameters to provide a best fit of the predicted enrichment kinetics of the one or more Aβ isoforms in the CNS to the measured kinetics from the patient. In an aspect, the model may output model parameters specific for each patient.

The concentrations of the one or more Aβ precursors and/or one or more Aβ isoforms and associated metabolic processes in the brain may be represented within the model. In one aspect, this representation within the model may include a compartment, a rate constant, flow equation, and/or any other mathematical representation known in the art without limitation. In an aspect, the concentration in a compartment may be calculated by multiplying the concentration in the previous compartment by a transfer rate constant between the two compartments minus any irreversible loss. Different aspects of the model may be differentiated by different numbers of compartments or types of compartments, the order of the compartments, the equations governing the trafficking and flow of Aβ isoforms, the Aβ isoform being modeled, or any other aspect for modeling the metabolism of a CNS biomolecule.

In another aspect, the kinetic model may represent the movement of soluble Aβ isoforms within the brain as a flow from the ventricles to the brain surface and into the CSF and/or blood. In an aspect, the movement of an Aβ isoform in the brain interstitial fluid (ISF) may be represented by at least one fluid flow equation. In another aspect, the flow of Aβ isoforms may be represented as a transfer between nodes distributed spatially between the point where the Aβ isoform may enter the ISF and the surface of the brain.

In an aspect, the concentration of a labeled moiety and measured concentrations of labeled Aβ isoforms in the CSF and/or blood may be used to develop a model of the metabolism of the labeled Aβ and to determine the rate constants associated with each compartment or flow equation. In addition, the model may be used to calculate predicted concentrations of the Aβ isoforms in the CSF, in the brain, in the blood, or at any other location in a patient. Non-limiting examples of how the model of in vivo Aβ metabolism may be used include identifying the disease state of a patient, fitting a curve of measured data acquired from a patient, predicting the metabolism, processing, and/or concentration of Aβ and its isoforms in a patient, identifying sensitive pathway components to help design drugs or understand a CNS disease, and investigating changes in the kinetics of the isoforms that may be induced by investigational drugs.

Detailed descriptions of various aspects of the methods of modeling the in vivo metabolism of Aβ are provided herein below.

### I. Methods of Developing a Model of the In Vivo Metabolism of Aβ

In various aspects, a method to develop a model to represent the synthesis of one or more Aβ isoforms in the central nervous system in vivo and to predict the turnover and production rates of the one or more Aβ isoforms in one or more patients is provided. Data from patients, including time course amounts of a labeled moiety and the concentration of at least one Aβ isoform, may be used in the development of the model.

### (a) Degenerative Diseases

In various aspects, the model may be used to predict the turnover and production rates of at least one Aβ isoform in a patient. In an aspect, the model may be used to predict the effects of the dysregulation of Aβ isoform turnover and production rates in a subject with Aβ amyloidosis. The term “Aβ amyloidosis” refers to Aβ deposition in a subject that may result from differential metabolism (e.g. increased production, reduced clearance, or both). Aβ amyloidosis is clinically defined as evidence of Aβ deposition in the brain either by amyloid imaging (e.g. PiB PET) or by decreased cerebrospinal fluid (CSF) Aβ42 or Aβ42/40 ratio. See, for example, Klunk W E et al. Ann Neurol 55(3) 2004, and Fagan A M et al. Ann Neurol 59(3) 2006, each hereby incorporated by reference in its entirety. Subjects with Aβ amyloidosis are also at an increased risk of developing a disease associated with Aβ amyloidosis. Diseases associated with Aβ amyloidosis include, but are not limited to, Alzheimer's Disease (AD), cerebral amyloid angiopathy, Lewy body dementia, and inclusion body myositis. Non-limiting examples of symptoms associated with Aβ amyloidosis may include impaired cognitive function, altered behavior, abnormal language function, emotional dysregulation, seizures, dementia, and impaired nervous system structure or function.

In another aspect, the model may be used to predict the effects of the dysregulation of Aβ isoform turnover and production rates resulting from a degenerative disease in a patient. Any degenerative disease characterized by the dysregulation in the turnover and production rate of any CNS biomolecule including, but not limited to at least one Aβ isoform may be predicted using the model without limitation. By way of non-limiting example, Alzheimer's Disease (AD) is a debilitating disease characterized by accumulation of amyloid plaques in the central nervous system resulting from increased production, decreased clearance, or a combination of increased production and decreased clearance of Aβ protein. While AD is an exemplary disease that may be diagnosed or monitored by various aspects of this disclosure, this disclosure is not limited to AD. It is envisioned that the method may be used in modeling the kinetics, diagnosis, and assessment of treatment efficacy of several neurological and neurodegenerative diseases, disorders, or processes including, but not limited to, AD, Parkinson's Disease, stroke, frontal temporal dementias (FTDs), Huntington's Disease, progressive supranuclear palsy (PSP), corticobasal degeneration (CBD), aging-related disorders and dementias, Multiple Sclerosis, Prion Diseases (e.g. Creutzfeldt-Jakob Disease, bovine spongiform encephalopathy or Mad Cow Disease, and scrapie), Lewy Body Disease, and Amyotrophic Lateral Sclerosis (ALS or Lou Gehrig's Disease). It is also envisioned that the method of modeling in vivo kinetics of a CNS disease may be used to study the normal physiology, metabolism, and function of the CNS.

The in vivo metabolism of at least one Aβ isoform or other CNS biomolecule may be modeled in any human patient without limitation. In one aspect, the human patient may be of an advanced age including, but not limited to, human patients older than about 85. Alternatively, the in vivo metabolism of CNS biomolecules may be modeled in other mammalian patients without limitation. In another aspect, the patient may be a companion animal such as a dog or cat. In another alternative aspect, the patient may be a livestock animal such as a cow, pig, horse, sheep or goat. In yet another alternative embodiment, the patient may be a zoo animal. In another aspect, the patient may be a research animal such as a non-human primate or a rodent.

### (b) Overview of Aβ Metabolism and Labeling

In various aspects, the architecture of the model may be developed using any known or hypothesized pathways and/or mechanisms of Aβ biometabolism without limitation.

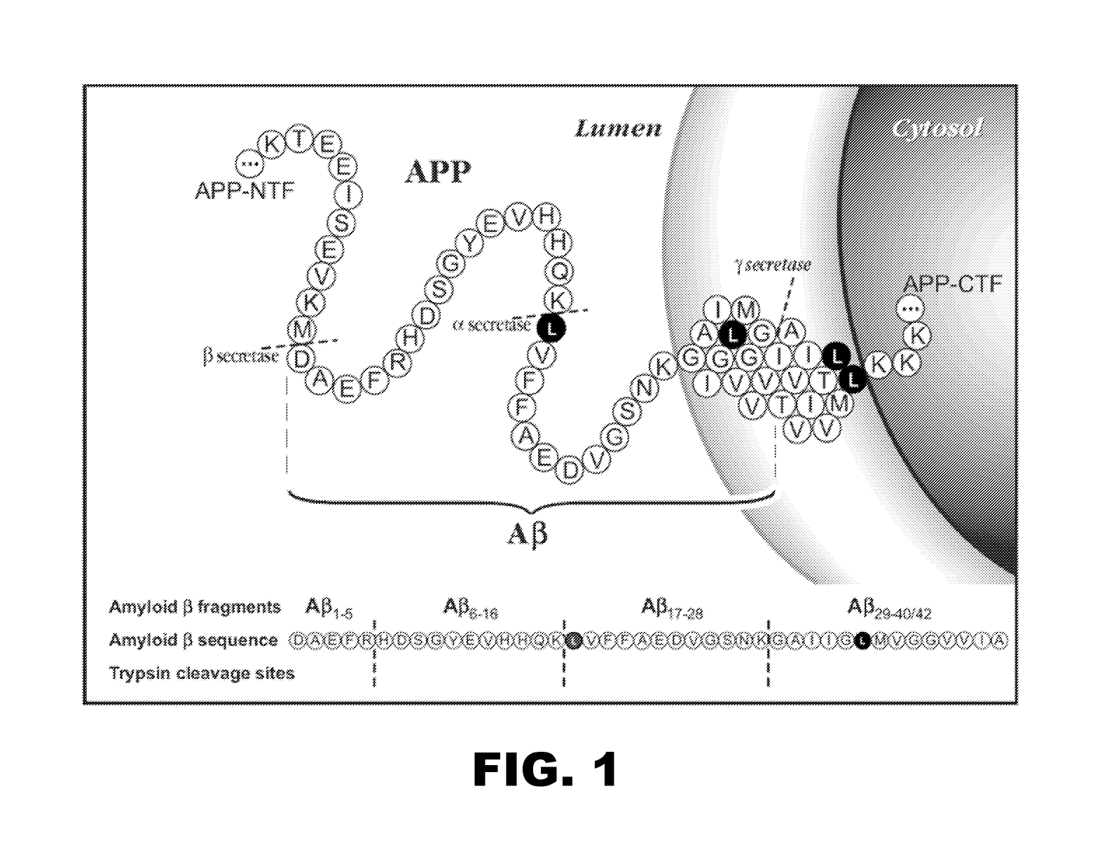

Without being limited to any particular theory, amino acids, including labeled amino acids, may be incorporated into amyloid precursor protein (APP) in neural cells. Amyloid precursor protein (APP) is a transmembrane protein expressed in many cells and may be concentrated in neurons and neuronal synapses. APP may be processed by α-, β-, and/or γ-secretases, creating peptides of varying length including, but not limited to, Aβ. C99 forms the c-terminal fragment of APP and is cleaved by the action of β-secretase. Aβ is a peptide of 36-43 amino acids located within the membrane-spanning domain of APP. Aβ is typically formed by the cleavage of APP by the β- and γ-secretases in succession or by the cleavage of C99 by α-secretase. γ-secretase includes enzymatic components PSEN1 and PSEN2. Varying isoforms of Aβ (e.g. Aβ38, Aβ40, Aβ42) may be produced through further processing and cleavage in the endoplasmic reticulum, the trans-Golgi network, or other areas of post-processing. FIG. 1 depicts a schematic illustrating the processing of APP into Aβ within a cell and indicates the locations where the secretases cleave APP. The amino acid sequence of Aβ (SEQ ID NO: 1) is shown at the bottom.

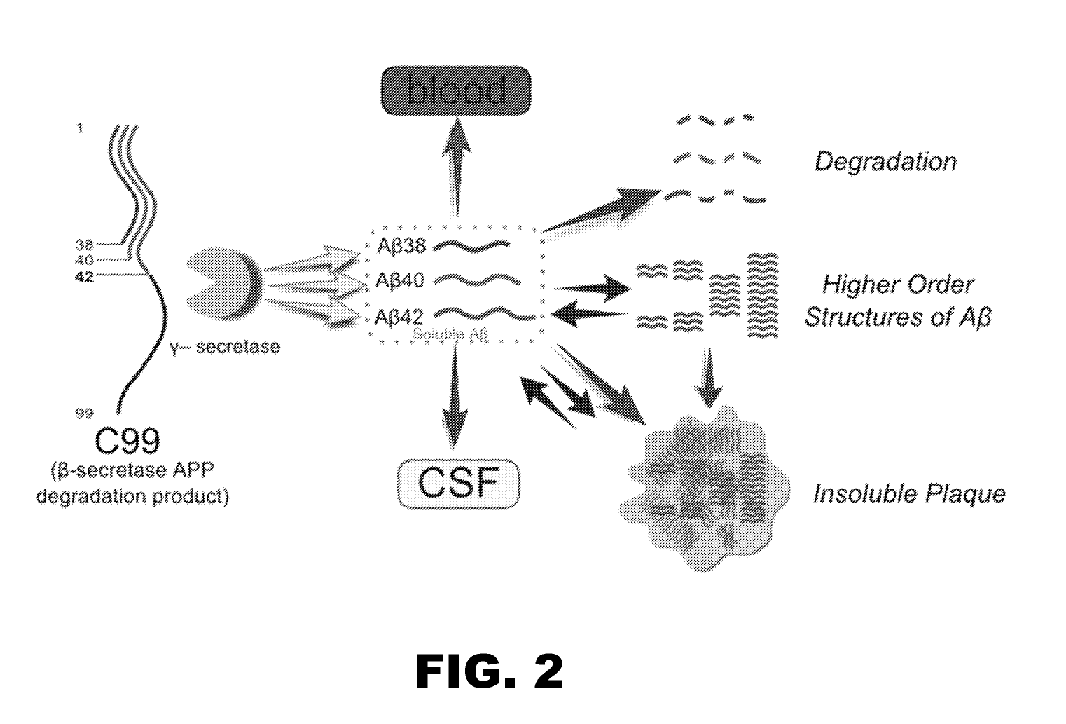

Because APP and C99 are cell-associated proteins, these proteins are not considered soluble and are not transported within the brain via flow mechanisms. However, after cleavage by α-secretase, Aβ peptides can flow within the brain's interstitial fluid (ISF). The Aβ peptides may be degraded within the brain, taken up in reversible higher order structures (e.g. micelles), taken up irreversibly into plaques, transported across the blood-brain barrier to the blood stream, and/or transported out of the brain as the ISF merges with the CSF, as illustrated in FIG. 2. There may be some recycling of the higher order structures and the plaques with the soluble Aβ isoform monomers, whereas degradation and exiting the blood brain barrier may irreversibly remove at least a portion of the soluble Aβ isoform monomers from the brain. ISF in the brain may be derived from the brain capillaries and from the ventricles. Without being limited to any particular theory, the pressure in the ventricles is typically higher than the pressure in the CSF, thereby inducing an outward flow of fluid from the ventricles to the surface of the brain and to the CSF.

To track the formation and kinetics of Aβ in vivo, newly formed APP may be labeled by incorporation of a labeled moiety during protein production. The labeled APP may then be cleaved into labeled Aβ isoforms. In an aspect, the labeled moiety may be an amino acid with a stable isotope of carbon, nitrogen, or any other isotope that may be incorporated into amino acids during protein production. Because leucine is more easily capable of crossing the blood brain barrier compared to other amino acids, leucine may be better-suited for use with CNS biomolecules and Aβ. Referring back to FIG. 1, labeled leucines (L) within Aβ are indicated in black.

### (c) CNS Biomolecule

The method for developing a model may include representing the metabolism of any biomolecule derived from the CNS in vivo including, but not limited to, at least one Aβ isoform. The CNS biomolecule may include, but is not limited to, a protein, a lipid, a nucleic acid, a carbohydrate, or any CNS biomolecule known in the art. Any CNS biomolecule may be represented, so long as the CNS biomolecule may be labeled during in vivo synthesis and a sample may be collected from which their metabolism may be measured. In an aspect, the CNS biomolecule is a protein synthesized in the CNS. Non-limiting examples of suitable proteins to be modeled include: amyloid-β (Aβ), Aβ isoforms and other variants, soluble amyloid precursor protein (APP), apolipoprotein E (isoforms 2, 3, or 4), apolipoprotein J (also called clusterin), Tau (another protein associated with AD), glial fibrillary acidic protein, alpha-2 macroglobulin, synuclein, S100B, Myelin Basic Protein (implicated in multiple sclerosis), prions, interleukins, TDP-43, superoxide dismutase-1, huntingtin, and tumor necrosis factor (TNF). Additional CNS biomolecules that may be targeted include products of, or proteins or peptides that interact with, GABAergic neurons, noradrenergic neurons, histaminergic neurons, seratonergic neurons, dopaminergic neurons, cholinergic neurons, and glutaminergic neurons.

The method may model the metabolism of APP in one aspect. In an additional aspect, the CNS biomolecule whose in vivo metabolism is modeled may be amyloid-beta (Aβ) protein. In another aspect, isoforms of Aβ (e.g., Aβ40, Aβ42, Aβ38 and/or others) may be modeled. In a further aspect, digestion products of Aβ (e.g., Aβ₆₋₁₆, Aβ₁₇₋₂₈) may be modeled. In an aspect, the model may represent the metabolism of more than one CNS biomolecule at a time. In one aspect, the CNS biomolecule may include, but is not limited to, C99, APP, Aβ38, Aβ40, Aβ42, and any other Aβ isoform.

### (d) Labeled Moiety

In an aspect, the plasma concentration of a labeled moiety may be input into the model. In one aspect, the labeled moiety plasma concentration may be used to develop the model and determine the model parameters.

When the method is employed to model the metabolism of a protein, the labeled moiety may be an amino acid. Those of skill in the art will appreciate that at least several amino acids may be used to provide the label of a CNS biomolecule. Generally, the choice of amino acid is based on a variety of factors such as: (1) the amino acid generally is present in at least one residue of the protein or peptide of interest; (2) the amino acid is generally able to quickly reach the site of protein synthesis and rapidly equilibrate across the blood-brain barrier; (3) the amino acid ideally may be an essential amino acid (not produced by the body), so that a higher percent of labeling may be achieved; (4) the amino acid label generally does not influence the metabolism of the protein of interest (e.g., very large doses of leucine may affect muscle metabolism); and (5) the relatively wide availability of the desired amino acid (i.e., some amino acids are much more expensive or harder to manufacture than others).

In an aspect, the amino acid leucine may be used to label proteins that are synthesized in the CNS. Non-essential amino acids may also be used; however, measurements may be less accurate. In one aspect, ¹³C₆-phenylalanine, which contains six ¹³C atoms, may be used to label a neurally derived protein. In an aspect, ¹³C₆-leucine may be used to label a neurally derived protein. In an exemplary aspect, ¹³C₆-leucine may be used to label amyloid-β.

There are numerous commercial sources of labeled amino acids, containing both non-radioactive isotopes and radioactive isotopes. Generally, the labeled amino acids may be produced either biologically or synthetically. Biologically produced amino acids may be obtained from an organism (e.g., kelp/seaweed) grown in an enriched mixture of ¹³C, ¹⁵N, or another isotope that is incorporated into amino acids as the organism produces proteins. The amino acids are then separated and purified. Alternatively, amino acids may be made using any known synthetic chemical processes. The labeled moiety may be administered to a patient using any one of at least several methods known in the art. Non-limiting examples of suitable methods of administration include intravenous, intra-arterial, subcutaneous, intraperitoneal, intramuscular, and oral administration. In one aspect, the labeled moiety is administered to the patient using intravenous infusion.

The labeled moiety may be administered slowly over a period of time or as a large single dose depending upon the type of analysis chosen (e.g., steady state or bolus). To achieve steady-state levels of the labeled CNS biomolecule, the labeling time generally should be of sufficient duration so that the labeled CNS biomolecule may be reliably quantified. The labeling time sufficient for reliable quantification of steady state levels of a labeled Aβ in a blood sample is typically less than required time for reliable quantification of steady state levels of Aβ in a CSF sample. See for example, U.S. Pat. No. 7,892,845 and U.S. Ser. No. 13/669,497, each hereby incorporated by reference in its entirety. This duration may be selected to be sufficient to result in saturation of the biochemical pathways associated with the synthesis of the CNS biomolecule. In one aspect, the duration may be sufficient to result in the saturation of the biochemical pathways associated with the synthesis and kinetics of at least one Aβ isoform in the brain of a patient, including, but not limited to: APP synthesis, cleavage of C99 and the at least one Aβ isoform, the transport of the at least one Aβ isoform to the CSF, and the transport of the at least one Aβ isoform to the blood. In another aspect, the saturation of the biochemical pathways may be indicated by the detection of stabilized levels of the at least one Aβ isoform in the CSF and/or blood as measured in a patient.

In an aspect, the labeled moiety is administered intravenously for an amount of time that is less than the half-life of Aβ in blood or CSF. In other aspect, the labeled moiety is administered intravenously for an amount of time that is greater than the half-life of Aβ in blood or CSF. For example, the labeled moiety may be administered intravenously over a duration of minutes to hours, including, but not limited to, for at least 10 minutes, at least 20 minutes, at least 30 minutes, at least 1.0 hour, at least 1.5 hours, at least 2.0 hours, at least 2.5 hours, at least 3.0 hours, at least 3.5 hours, at least 4.0 hours, at least 4.5 hours, at least 5.0 hours, at least 5.5 hours, at least 6.0 hours, at least 6.5 hours, at least 7.0 hours, at least 7.5 hours, at least 8.0 hours, at least 8.5 hours, at least 9.0 hours, at least 9.5 hours, at least 10.0 hours, at least 10.5 hours, 1 at least 1.0 hours, at least 11.5 hours, or at least 12 hours. In another aspect, the labeled moiety may be administered intravenously over a period ranging from about 6 hours to about 18 hours. In another aspect, the labeled moiety may be administered intravenously over a period of about 9 hours. In another aspect, the labeled moiety may be administered intravenously over a period of about 3 hours. In yet another aspect, a labeled moiety is administered orally as multiple doses. The multiple doses may be administered sequentially or an amount of time may elapse between each dose. The amount of time between each dose may be a few seconds, a few minutes, or a few hours. In each of the above embodiments, the labeled moiety can be labeled leucine, labeled phenylalanine, or any other labeled amino acid that is capable of crossing the blood-brain barrier.

Those of skill in the art will appreciate that the amount (or dose) of the labeled moiety can and will vary. Generally, the amount is dependent on (and estimated by) the following factors. (1) The type of analysis desired. For example, to achieve a steady state of about 15% labeled leucine in plasma requires about 2 mg/kg/hr over 9 hr after an initial bolus of about 3 mg/kg over 10 min. In contrast, if no steady state is required, a bolus of labeled leucine (e.g., about 400 mg to about 800 mg of labeled leucine) may be given. (2) The Aβ variant under analysis. For example, if the Aβ variant is being produced rapidly, then less labeling time may be needed and less label may be needed—perhaps as little as 100 mg or less as a bolus. And (3) the sensitivity of the technology to detect label. For example, as the sensitivity of label detection increases, the amount of label that is needed may decrease.

In another aspect, a labeled moiety is administered orally as a single bolus. In another aspect, a labeled moiety is administered intravenously as a single bolus. In still another aspect, a labeled moiety is administered intravenously as an infusion for about 1 hour. All three methods of administration (oral bolus, IV bolus, and IV infusion) work equally well in terms of providing a reliable measure of amyloid beta metabolism. An intravenous bolus of a labeled moiety and an oral bolus of labeled moiety are easier to administer than an intravenous infusion, and also results in maximal levels of free label at an earlier time point (e.g. about 5 to about 10 minutes, and about 30 to about 60 minutes, respectively, for labeled leucine). In each of the above embodiments, the labeled moiety can be labeled leucine, labeled phenylalanine, or any other labeled amino acid that is capable of crossing the blood brain barrier.

### (e) Biological Sample

The method of developing the model may include obtaining a biological sample from a patient so that the in vivo metabolism of the labeled CNS biomolecule may be determined. Information from a patient's biological sample may be used as an input in the method of developing and/or calibrating a model of in vivo metabolism of a CNS biomolecule.

Suitable biological samples include, but are not limited to, cerebral spinal fluid (CSF), blood plasma, blood serum, urine, saliva, perspiration, and tears. In one aspect, biological samples may be taken from the CSF. In an alternate aspect, biological samples may be collected from the urine. In another aspect, biological samples may be collected from the blood.

Cerebrospinal fluid may be obtained by lumbar puncture with or without an indwelling CSF catheter (a catheter is preferred if multiple collections are made over time). Blood may be collected by veni-puncture with or without an intravenous catheter. Urine may be collected by simple urine collection or more accurately with a catheter. Saliva and tears may be collected by direct collection using standard good manufacturing practice (GMP) methods.

In general, when the CNS biomolecule is a protein, the method of developing and/or calibrating the model may include obtaining a first biological sample to be taken from the patient prior to administration of the labeled moiety to provide a baseline for the patient. After administration of the labeled amino acid or protein, one or more samples generally may be taken from the patient. As will be appreciated by those of skill in the art, the number of samples and when they may be taken generally depend upon a number of factors such as: the type of analysis, type of administration, the protein of interest, the rate of metabolism, the type of detection, etc.

In general, samples obtained during the labeling phase may be used to determine the rate of synthesis of the Aβ variant, and samples taken during the clearance phase may be used to determine the clearance rate of the Aβ variant. Labeled Aβ increases during labeling and then decreases after the labeling has stopped. In one aspect, the CNS biomolecule may be a protein including, but not limited to at least one Aβ isoform and one or more samples of CSF may be taken hourly for 36 hours. Alternatively, the samples may be taken every other hour or even less frequently. Typically, biological samples obtained during the first 12 hours of sampling (i.e., 12 hrs after the start of labeling (IV bolus or infusion) may be used to determine the rate of synthesis of the protein, and biological samples taken during the final 12 hours of sampling (i.e., 24-36 hrs after the initial infusion of labeled moieties) may be used to determine the clearance rate of the protein. In another aspect, a single sample may be taken after labeling for a period of time, such as 12 hours, to estimate the synthesis rate, but this may be less accurate than multiple samples. In another aspect, the CNS biomolecule may be a protein including, but not limited to at least one Aβ isoform and one or more samples of blood may be taken hourly for 24 hours. Alternatively, the samples may be taken every other hour or even less frequently. Typically, blood samples obtained during the first 4 hours of sampling (i.e., about 1 minute to about 4 hrs after administration of an IV or oral bolus, about 10 minutes to about 4 hrs after administration of an IV or oral bolus, about 30 minutes to about 4 hrs after administration of an IV or oral bolus, about 1 minute to about 3 hrs after administration of an IV or oral bolus, about 10 minutes to about 3 hrs after administration of an IV or oral bolus, or about 30 minutes to about 3 hrs after administration of an IV or oral bolus) may be used to determine the rate of synthesis of the protein, and blood samples taken during the final 20 hours after administration of an IV or oral bolus (i.e., about 4 hours to about 12 hours after administration of an IV or oral bolus, about 12 hours to about 24 hours after administration of an IV or oral bolus, about 18 hours to about 24 hours after administration of an IV or oral bolus, or about 4 hours to about 24 hours after administration of an IV or oral bolus) may be used to determine the clearance rate of the protein. In another aspect, a single sample may be taken after administration of an IV or oral bolus, such as at about 3 hours, to estimate the synthesis rate, but this may be less accurate than multiple samples. In yet a further aspect, samples may be taken from an hour to days or even weeks apart depending upon the protein's synthesis and clearance rate.

### (f) Developing a Model

The method of developing a kinetic model may include developing a model that may fit experimental findings in a manner consistent with known molecular biology and physiologic structures. In an aspect, the kinetic model may be a comprehensive steady state compartmental model that uses tracer kinetics to determine the rate constants within the model. In another aspect, the model may account for the time course of at least one Aβ isoform in vivo. In yet another aspect, the model may mathematically represent the one-dimensional flow of soluble Aβ isoforms in the brain from the ventricles to the CSF and/or blood. In this aspect, the flow may be due to the pressure difference between the ventricles and the brain surface.

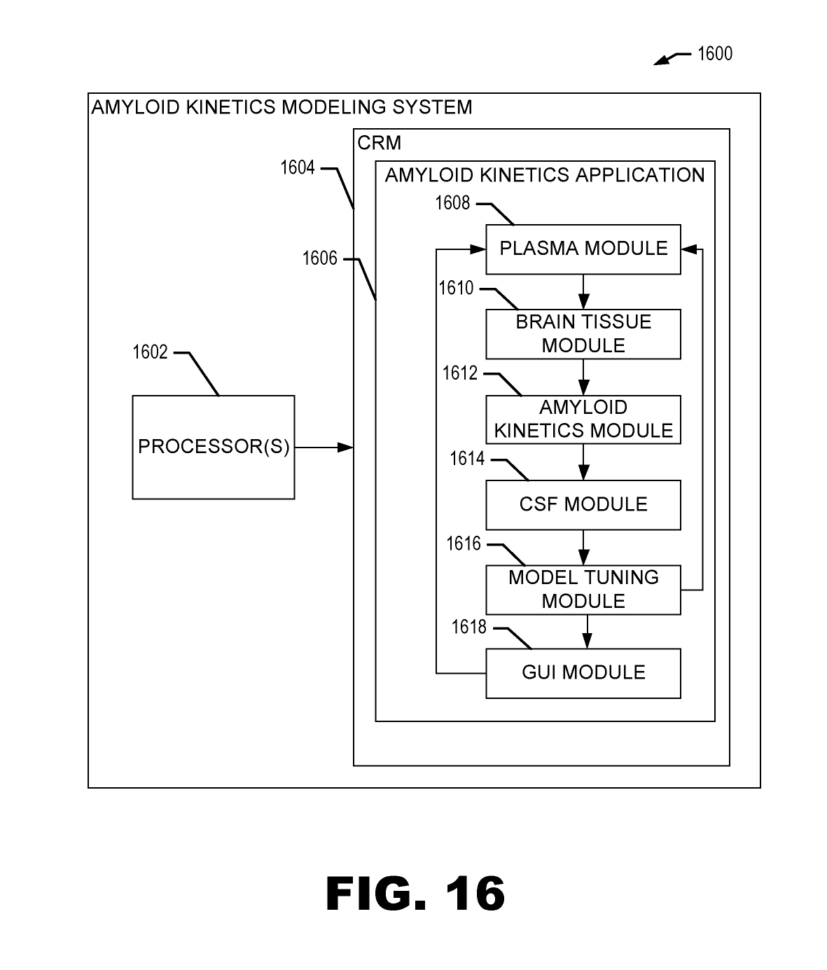

FIG. 16 is a block diagram of an amyloid kinetics modeling system 1600 in one aspect. The amyloid kinetics modeling system 1600 may include one or more processors 1602 and a machine-readable or computer-readable medium (CRM) 1604 containing an amyloid kinetics application 1606. The amyloid kinetics application 1606 includes a plurality of modules executable on the one or more processors 1602.

The plasma module 1608 represents the infusion of the labeled moiety into the plasma of a patient and the transport of the labeled moiety across the blood brain barrier (BBB). The brain tissue module 1610 represents the incorporation of the labeled moiety into APP and the formation of C99. The amyloid kinetics module 1612 represents the cleavage of the C99 to form at least one Aβ isoform and the subsequent kinetics of the at least one Aβ isoform within the brain including, but not limited to, recycling, fractional turnover, incorporation into plaques, transport across the blood brain barrier (BBB), and breakdown of the at least one Aβ isoform. The CSF module 1614 represents transport of the at least one Aβ isoform into the CSF. The model tuning module 1616 may iteratively adjust a set of parameters defining the dynamic response of the model to the input time history of plasma leucine enrichment into the plasma module 1608 in order to optimize the match between the predicted CSF enrichment kinetics and the measured CSF enrichment kinetics of the at least one Aβ isoform in the patient.

In an aspect, the amyloid kinetics application 1606 may further include a blood enrichment module (not shown). The blood enrichment module represents transport of the at least one Aβ isoform into the blood. In an additional aspect, the amyloid kinetics application 1606 may include the blood enrichment module in the place of the CSF module 1614.

The GUI module 1618 may generate one or more forms to receive inputs to the system 1600 such as the time history of plasma leucine enrichment and the measured CSF enrichment kinetics of the at least one Aβ isoform in the patient. The GUI module 1618 may further receive additional user inputs such as defined ranges for parameters defining the dynamic response of the model and other values used to specify the operation of the system 1600. The GUI module 1618 may also generate one or more forms used to display outputs of the application 1606 including, but not limited to graphs of the predicted CSF enrichment kinetics of the at least one Aβ isoform, listings of model parameters, predictions of a disease state of a patient, and any other relevant output.

Any method of modeling may be used to implement any one or more of the modules 1608-1614 without limitation. Non-limiting examples of suitable modeling methods include compartmental models, flow models, mathematical equations, fluid dynamic flow equations, diffusion equations, any other suitable modeling method known in the art. In one aspect, the modules 1608-1614 may be implemented using compartmental models. In another aspect, the modules 1608-1614 may be implemented using a combination of compartmental models and flow models.

(i) Compartmental Model

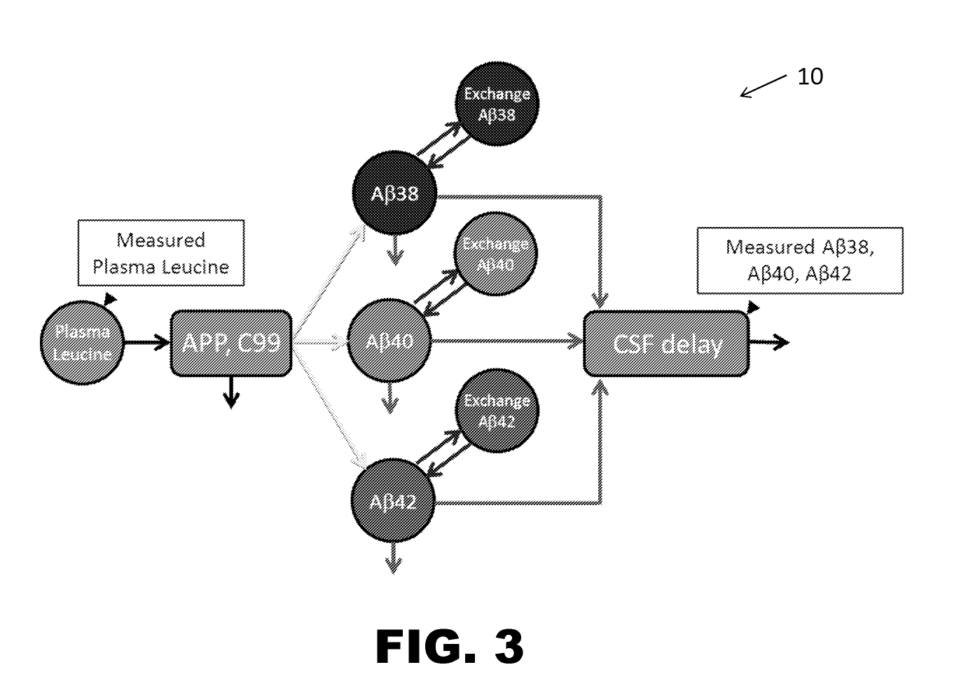

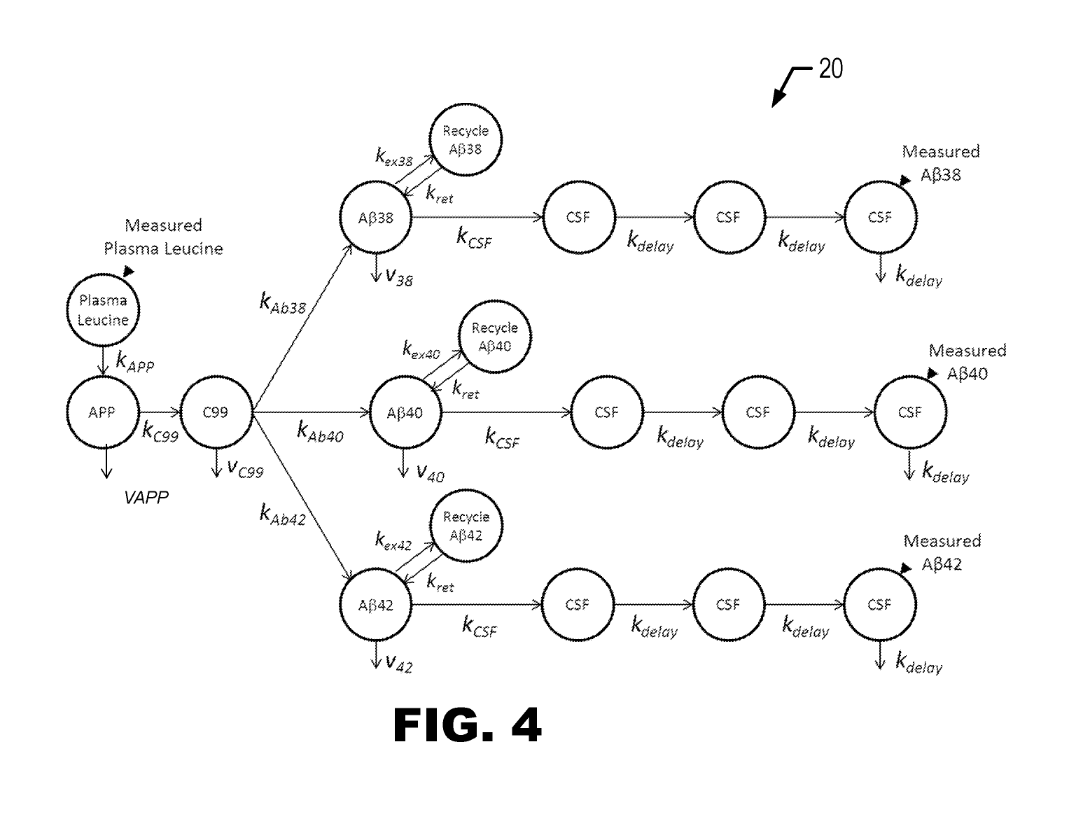

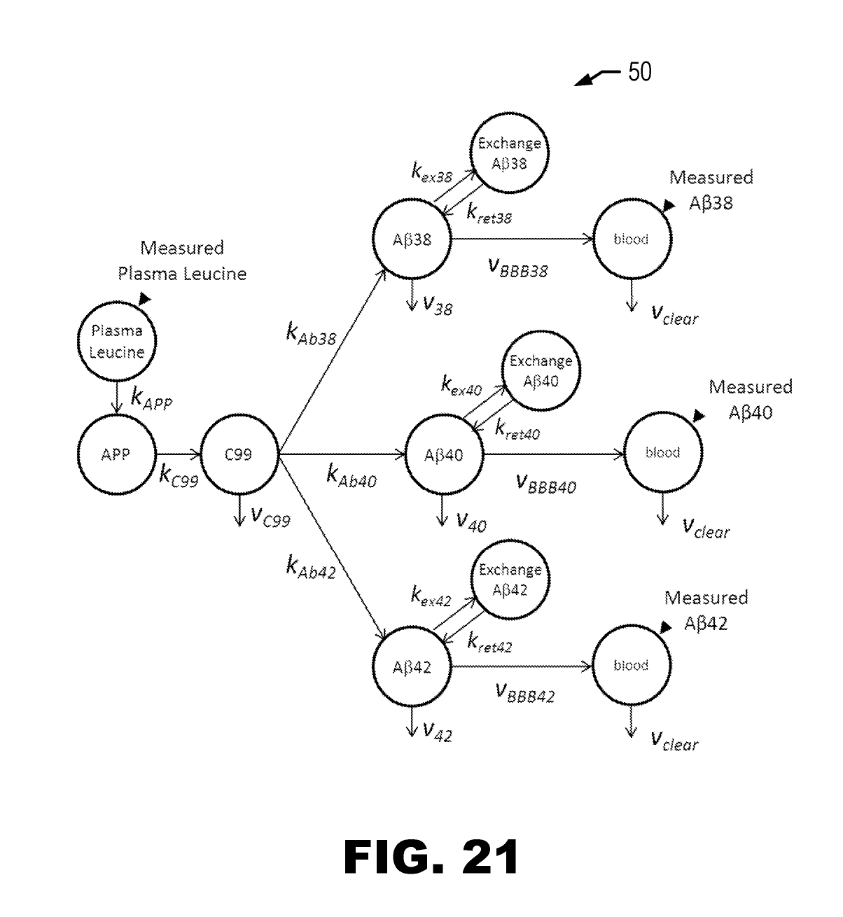

FIG. 3 is a schematic diagram showing the overall architecture of a model 10 of Aβ kinetics using a compartmental model in an aspect. FIG. 4 is a diagram of the full architecture of a model 20 of Aβ kinetics using a compartmental model in another aspect. In this other aspect, the model 20 may include a series of interconnected compartments with first order rate constants that describe the transfer of labeled species between compartments. The compartments may represent different forms of Aβ or different locations of Aβ isoforms along a metabolic pathway. FIG. 21 is a schematic diagram showing the overall architecture of an additional model 50 of Aβ kinetics using a compartmental model in an aspect

The kinetic model may account for the full time course of Aβ38, Aβ40, and Aβ42 enrichments and CSF concentrations in one aspect. In an aspect, the model may describe fundamental processes that affect Aβ kinetics including, but not limited to: production, reversible exchange, and irreversible loss, and may account for the effect of the kinetics of these processes on CSF concentrations of Aβ.

The model may be implemented on any software or device without limitation. In an aspect, modeling may be performed using SAAM II software (Resource for Kinetic Analysis, University of Washington, Seattle). In various aspects, the number, order, and location of compartments may vary. In various other aspects, the interconnections between the various compartments may vary. In various additional aspects, functions other than first-order rate constants may be used to represent the movement of a quantity from one compartment to another. Non-limiting examples of suitable functions include linear functions, exponential functions, differential equations, logarithmic equations, and any other known kinetic and/or rate equation known in the art. The functions may be constant with respect to other variables within the model, or the functions may include other variables generated within the model. For example, the rate of synthesis of an Aβ isoform may be influenced by the concentration of soluble Aβ isoform already produced in an aspect.

The kinetic model may include a compartment for the concentration of a labeled moiety. In one aspect, the kinetic model may include a compartment for labeled plasma leucine. In another aspect, the kinetic model may include at least one compartment for APP. In other aspects, the kinetic model may include compartments for iAPP and mAPP. In yet another aspect, the kinetic model may include a compartment for C99. The kinetic model may include parallel arms for different CNS biomolecules or Aβ isoforms. In an aspect, the kinetic model may include three parallel arms with corresponding compartments, one for each Aβ isoform (Aβ42, Aβ40, Aβ38), as illustrated in FIG. 4. In another aspect, the kinetic model may include a reversible exchange compartment for at least one Aβ isoform. In one aspect, the kinetic model may include a reversible exchange compartment for Aβ42. In other aspects, the kinetic model may include at least one delay compartment for the transport of the Aβ isoforms from the brain to the CSF. The compartments may be connected by rate constants for the rate of transfer from one compartment to the next. In yet another aspect, the model may account for irreversible loss of C99 and each soluble Aβ isoform that may not be recovered in the CSF.

The method of developing the kinetic model may include acquiring data from various patients to input into the development of the model. In one aspect, the enrichment of the labeled moiety and labeled Aβ isoform peptides may be measured at frequent time intervals (indicated by solid triangles in FIGS. 3, 4, and 21). In an aspect, the labeled moiety may be plasma ¹³C₆-leucine. In another aspect, the measured values for each patient may be used to optimize the parameters of the model for each patient. The model parameter values may be averaged for each patient type or disease state including, but not limited to non-carriers/normal controls (NC), mutation carriers (MC) PIB−, mutation carriers PIB+, and other neurological disease states.

Referring to FIG. 4, the model may include, but is not limited to, compartments for plasma leucine, APP, C99, Aβ38, Aβ40, Aβ42, CSF/delay, recycling, and any other compartment that may be necessary to model the metabolism of Aβ. In an aspect, a “forcing function” may be used to describe the time course of plasma ¹³C₆-leucine enrichment using a linear interpolation of ¹³C₆-leucine enrichment between measured plasma samples. Each Aβ isoform may be optimally described by a single turning over compartment coupled with a long time delay that may include one or more sub-compartments. In an aspect, delay compartments representing APP and C99 peptides may be placed in front of the compartments that represent the brain “soluble” Aβ peptides. Without being limited to any particular theory, these delay compartments may be added because in vivo tracer studies in mice indicated that APP and C99 have relatively long half-lives (about 3 hours) that should contribute to the overall time delay before labeled Aβ is detected at the lumbar sampling site. Other compartments may be placed after the “soluble” Aβ compartments to represent perfusion of labeled peptides through brain tissue, flow within the ISF, and heterogeneous CSF fluid transport processes. Since preliminary modeling indicates that a single time delay process could be identified within the data, the turnover rates APP, C99, and each of the three CSF delay compartments may be set to a single adjustable parameter that affects the overall time delay in an aspect.

The kinetic model may take into consideration that some of the C99 and soluble Aβ peptides may be metabolized to fates other than Aβ peptides that appear at the CSF sampling site in an aspect. Without being limited to any particular theory, the physiologic nature of these other losses for soluble Aβ peptides may be unknown at this time, but the model may include all processes that remove soluble peptides irreversibly, e.g. deposition into plaques, cellular uptake, proteolytic degradation, and/or transfer into the blood. In an aspect, the model may include an irreversible loss of each soluble Aβ isoform that was not recovered in CSF.

In an aspect, a reversible exchange compartment in exchange with the “soluble” Aβ peptide may be added to the model to optimally fit the sigmoid shape of the CSF Aβ enrichment time courses after the peak enrichment. The reversible exchange may represent possible recycling of Aβ isoforms to and/or from plaques, the exchange of labeled Aβ for unlabeled Aβ, the recycling of higher order Aβ structures, or any other reversible exchange of Aβ. In one aspect, a reversible exchange compartment may be included for Aβ42. In an aspect, the exchange process may only be added for an isoform if it improves the Akaike Information Criteria (AIC) of the fit as provided by SAAM II software.

In another aspect, a scaling factor (SF) may be applied to each of the Aβ isoform enrichments after the kinetic model has first been developed if it improves the AIC. Without being limited to any particular theory, the SF may account for small amounts of isotopic dilution between plasma leucine and the biosynthetic precursor pool (generally less than about 5%) or to correct for minor calibration errors (generally less than about 10%) in the measurement of isotope enrichments of plasma leucine and/or Aβ peptides.

One principle parameter obtained with the model is the fractional turnover rate (pools/h) of the “soluble” Aβ peptides, i.e. the sum of the fractional rate of loss of these compartments to CSF and other losses from the system. Based on the calibrated kinetic parameters that describe the shape and magnitude of the CSF Aβ enrichment time course, the model may determine the rate constant (pools/h) for production of each Aβ peptide isoform from their common C99 precursor to accurately project the measured baseline CSF Aβ peptide concentrations. The model may project the steady state masses (ng) within and the flux rates (ng/h) between all compartments for each Aβ isoform.

The rate constants for transfer between compartments in the model may be calibrated for each patient by utilizing the labeled moiety time course and the measured time course of the Aβ isoforms in the biological sample. The model parameters to be calibrated may include, but are not limited to, transfer rate constants for APP, C99, Aβ38, Aβ40, and Aβ42; irreversible loss rate constants for C99, Aβ38, Aβ40, Aβ42, and CSF; exchange rate constants for Aβ38, Aβ40, and Aβ42; return rate constants; delay rate constants; and scaling factors. In another aspect, a database, similar to the data source shown and described below with reference to FIG. 9, and containing one or more optimal rate constants may be created. In one aspect, the calibrated rate constants may be obtained by developing an optimal model for each patient with a disease state. The database may also include values for all other necessary model parameters for a particular CNS biomolecule or Aβ isoforms for both the normal and various disease states. In an aspect, the model parameters and database may be used to calculate a model index and threshold respectively, as described herein below. As such, the values within the database may be used to identify a patient's disease state or predict and/or calibrate the kinetic model of desired CNS biomolecules in future patients, as discussed herein below.

Referring to FIG. 21, at least a fraction of Aβ isoforms in the brain may be transferred to the blood stream, generally across the blood brain barrier (BBB) in another aspect. In this other aspect, clearance from the brain, represented by V₃₈, V₄₀ and V₄₂, may include degradation and transfer to the CSF, while vBBB₃₈, vBBB₄₀ and vBBB₄₂ represent clearance to the blood or plasma of Aβ 38, Aβ 40 and Aβ 42, respectively. The blood/plasma mathematical model 50 may be fit to isotope enrichment data of Aβ isoforms collected from blood/plasma using the same methodology by which the CSF mathematical model is used to fit isotope enrichment data of Aβ isotopes collected from the CSF.

In an aspect, the model may include a representation of transfers of at least a fraction of the Aβ isoforms in the brain to the CSF. In an aspect, the model may include a representation of transfers of at least a fraction of the Aβ isoforms in the brain to the blood. In an additional aspect, the model may include a representation of transfers of at least a fraction of the Aβ isoforms in the brain to the CSF as well as a representation of transfers of at least an additional fraction of the Aβ isoforms in the brain to the blood.

In various aspects, the architecture of the model may be developed using the data measured from various patients as described above. The results of alternative model architectures that may vary in the number, order, location, and/or interconnections between compartments may be compared using a figure of merit, and the model architecture associated with the most favorable figure of merit may be selected. Non-limiting examples of suitable figures of merit include Akaike information criterion, Bayesian information criterion, Deviance information criterion, Focused information criterion, Hannan-Quinn information criterion, and any other suitable figure of merit known in the art.

Those skilled in the art will recognize that the order of the compartments in a linear model does not affect the fit of the data or the values of the determined parameters. Those skilled in the art will also recognize that some distinct rate constants in these mathematical models may be set to the same value in some cases where the individual parameters are unidentifiable or poorly identified by the data. The impact of these small changes to the structure of the model on the values of the rate constants may typically be minimal.

(ii) Flow Model

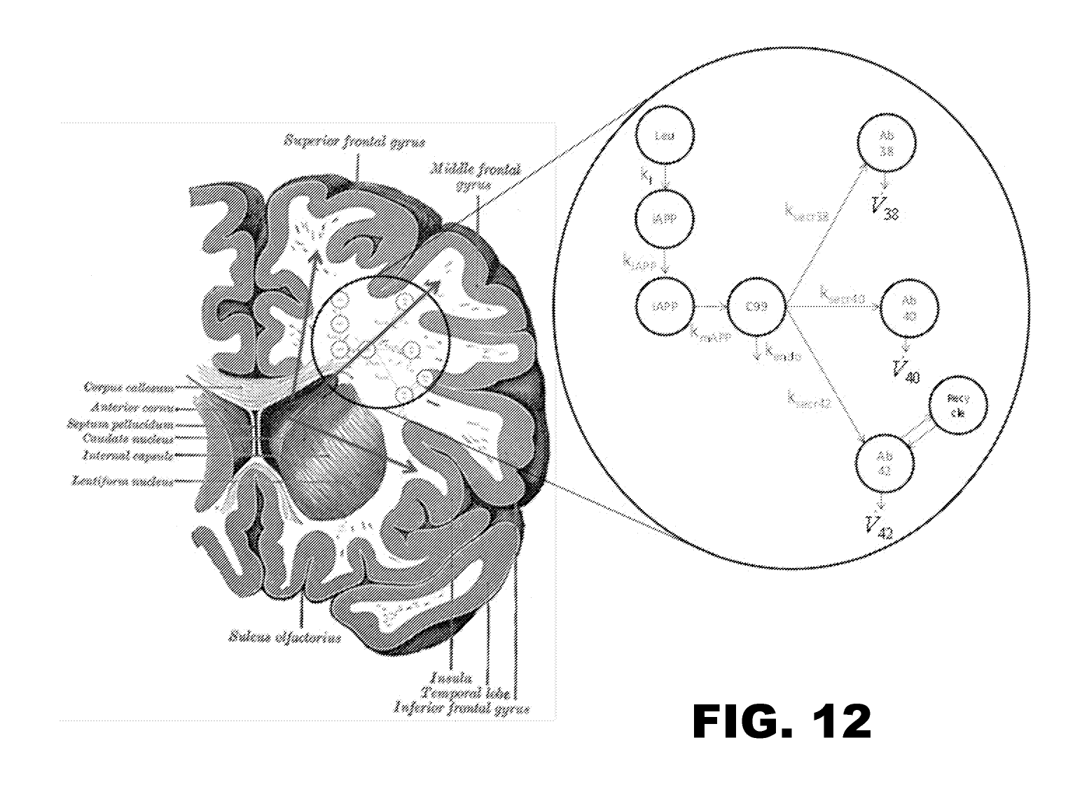

In an aspect, the kinetic model may be a flow model. FIG. 12 is a diagram of the architecture of a model of Aβ kinetics using a flow model in one aspect. In an aspect, the flow model may include any compartments or transfer rates from the compartmental model described above. In another aspect, the flow model may be used in combination with the compartmental model.

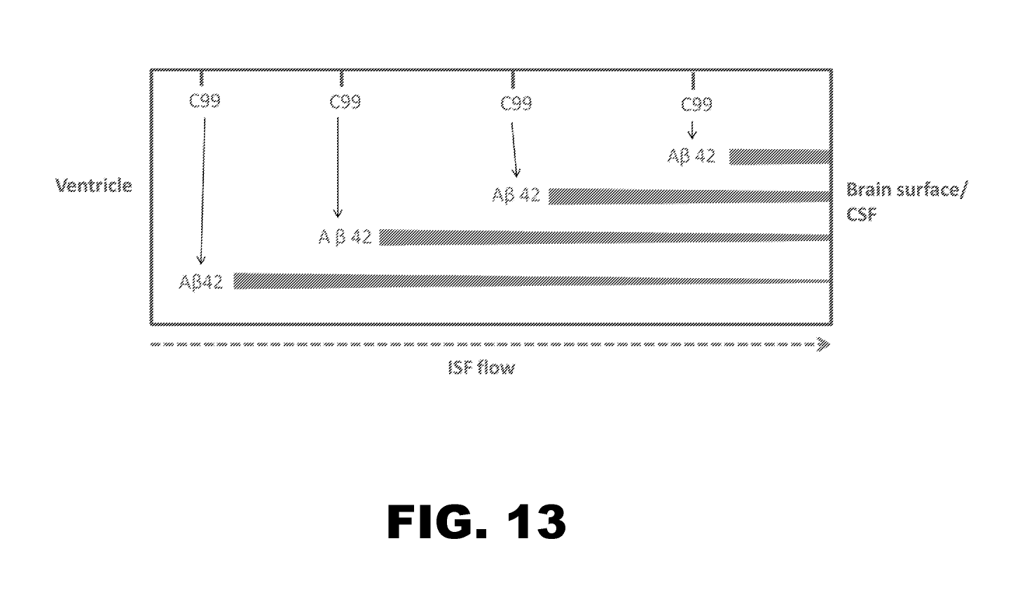

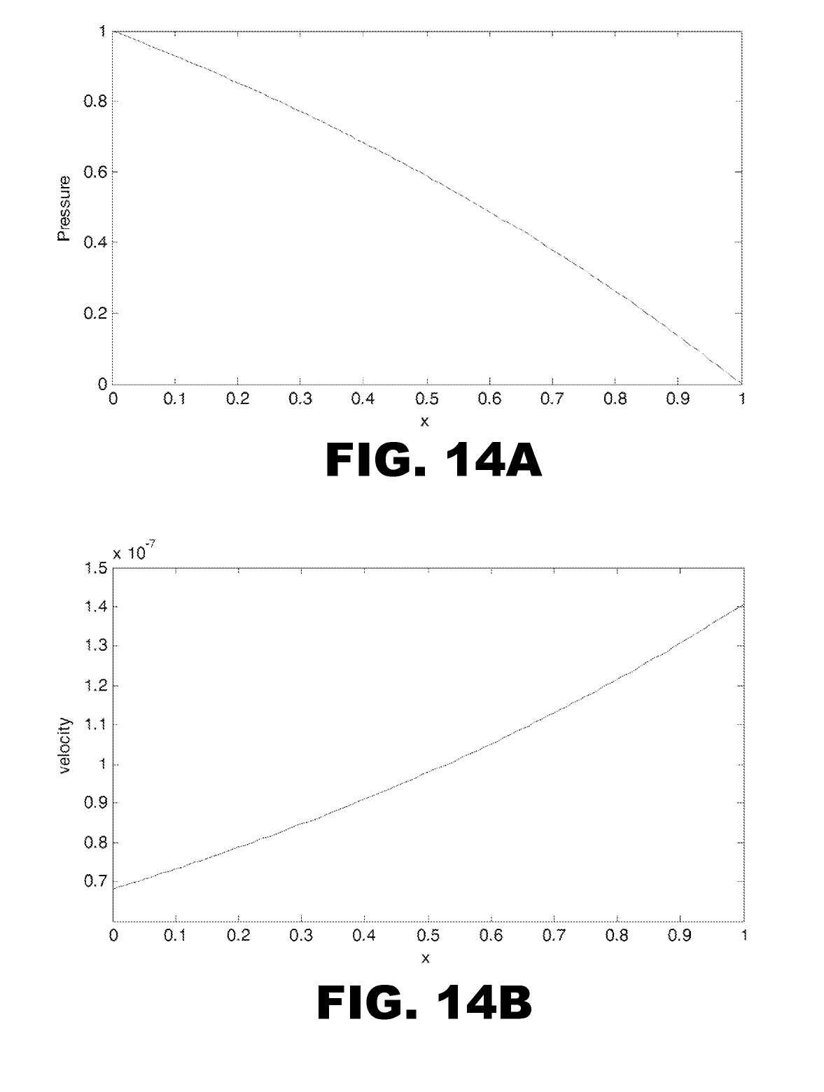

The kinetic model may account for one-dimensional flow of Aβ isoforms in the ISF of the brain from the ventricles to the brain surface and into the CSF through a pressure differential as illustrated in FIGS. 13 and 14A. In an aspect, a continuity equation and momentum balance of ISF in the brain may be used to model the flow of the Aβ isoforms in the flow model. In another aspect, the steady state flow of Aβ within the brain may be calculated. In an additional aspect, the flow of Aβ may be described by the equations in Example 4 herein below. Implementation of a full 3D flow model may be developed using 3D structural MRI data in another additional aspect.

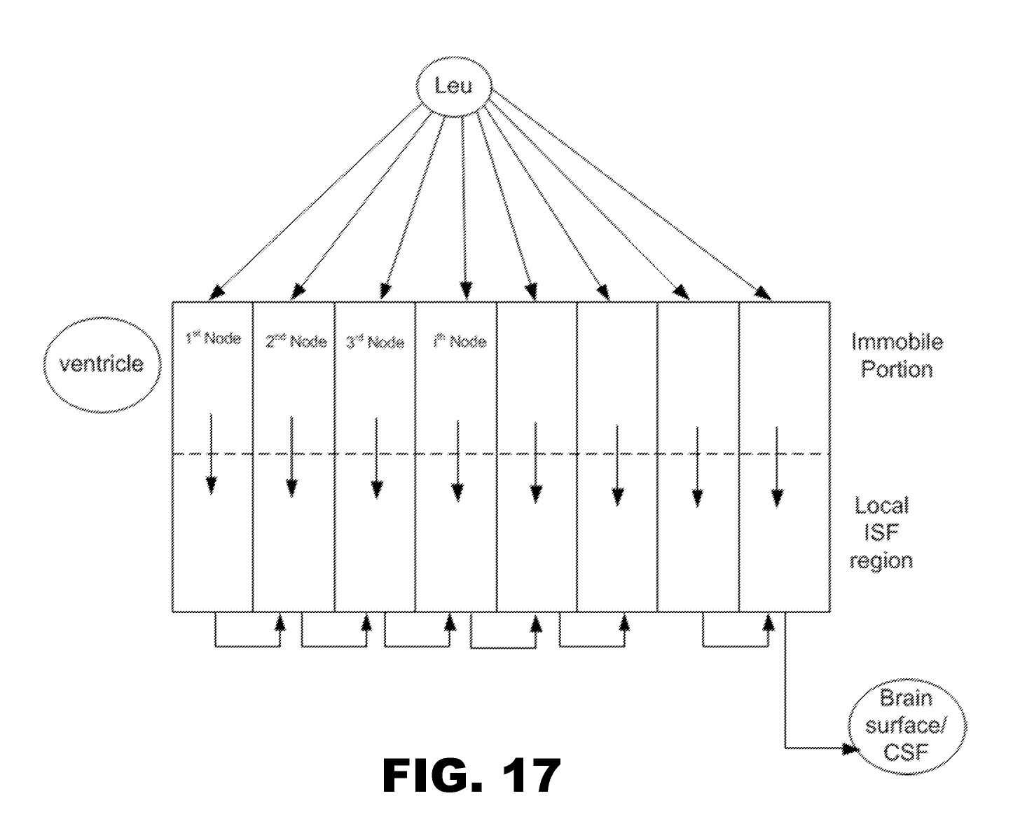

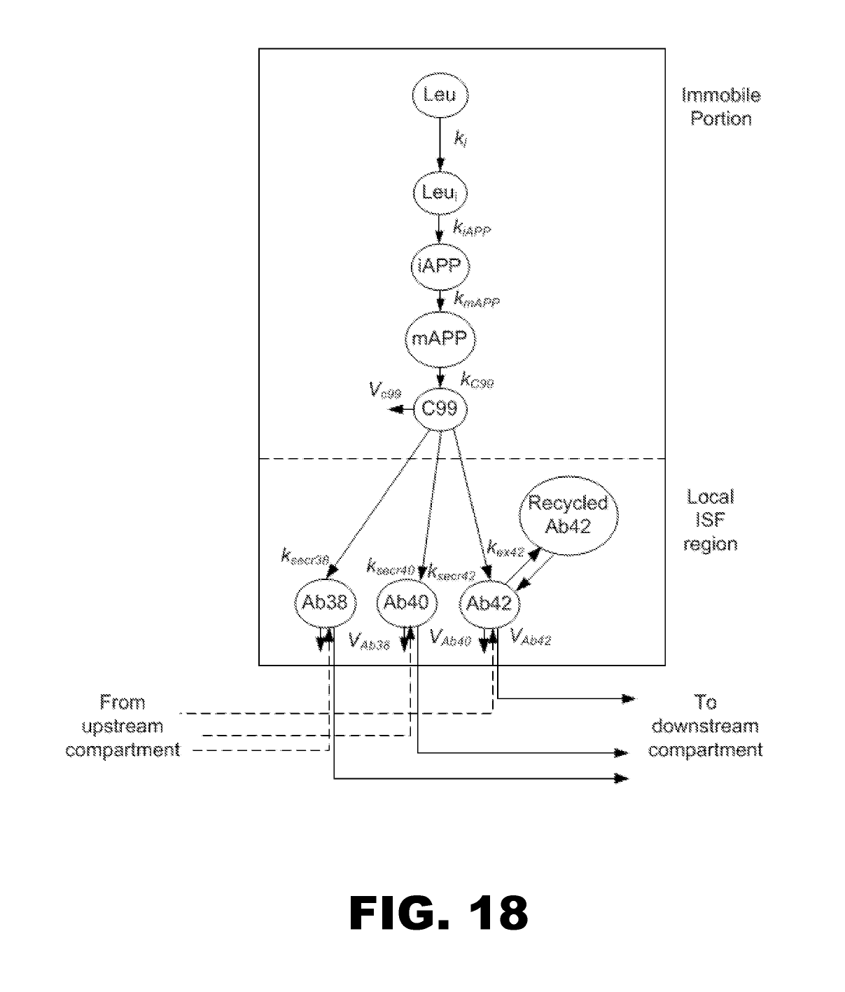

In an aspect, Illustrated in FIG. 17, the kinetic model may include nodes to represent the movement of the Aβ isoforms from the brain ventricles to the surface of the brain. Each node may be situated at a distance x from the ventricle (x=0) to the surface of the brain or CSF (x=1) associated with a local region of ISF. The ISF may move through each local region at a velocity prescribed by a computed velocity profile, summarized in one aspect in FIG. 14B. Within each local region, illustrated in FIG. 18, Aβ may be removed by exchange or irreversible loss and Aβ may be added by synthesis by the tissues in contact with the ISF in the immobile portion within that node. In one aspect, the kinetic model may include about 100 nodes for each Aβ isoform. The flow model may be represented by a plasma leucine compartment that is then divided into each node, as illustrated in FIG. 18.

Each node may be divided into an immobile and mobile portion, with the immobile portion remaining at that location in the brain and the mobile portion moving toward the surface of the brain at a velocity that may be derived from the computed velocity summarized in FIG. 14B. Referring back to FIG. 18, the immobile portion may include the compartments and transfer rates for Leucine, APP, iAPP, mAPP, C99, or any Aβ isoform in an exchange compartment. The mobile portion may include concentrations, irreversible loss, and flow rates for at least one soluble Aβ isoform.

The irreversible loss of each Aβ isoform may occur simultaneously with the flow of the Aβ isoform in the ISF. The movement of an Aβ isoform at any node or position within the ISF may be described in terms of flow and reaction. The reactions may be defined by the production of the Aβ isoform from C99, the degradation of the Aβ isoform (irreversible loss), and the exchange of the Aβ isoform with immobile forms of the Aβ isoform. In an aspect, each Aβ isoform may be tracked spatially in one dimension and the addition and removal of the Aβ isoform may be accounted for at each x location.

In another aspect, the flow may be incorporated into a compartment or rate constant within the compartmental model. The kinetic model may account for three-dimensional flow of Aβ in one aspect.

### II. Methods for Modeling the In Vivo Metabolism of Aβ

In various aspects, the methods for modeling the in vivo metabolism of at least one CNS biomolecule may be performed on one or more processing systems having one or more processors. In an aspect, the CNS molecule may be Aβ or an Aβ isoform. In one aspect, an Aβ modeling calibration system provides one or more graphical user interfaces that enable users to selectively calibrate a modeling system to identify, track, and estimate amounts or levels of a particular Aβ isoform or labeled protein segments at various time points in the metabolic pathway of Aβ. The Aβ modeling calibration system may be used to refine and calibrate a kinetic model for estimating amounts of Aβ lost to: degradation, formation of higher order structures and insoluble plaques, or Aβ otherwise transported to the blood or CSF. The Aβ modeling calibration system may therefore be used to calibrate a model for determining or predicting the fractional turnover rate of the “soluble” Aβ peptides (pools/h). In particular, by comparing model-derived data with known data values stored in memory, in a database, or in any other data storage medium, the system 100 may be used to calibrate the kinetic parameters, also stored in memory, for predicting various rate constants for the metabolism of Aβ peptides based on the measured baseline CSF Aβ peptide concentrations. As previously described, the CNS Aβ modeling calibration system 100 may be used to calibrate the optimal rate constants for the transfer between the various compartments in the kinetic models 10, 20, and 50 by comparing measured labeled moiety concentrations and the measured concentrations of the Aβ isoforms in a biological sample. Moreover, the system 100 may determine or predict the steady state masses (ng) within and the flux rates (ng/h) between the compartments of the model, as shown in FIGS. 3, 4, and 20 for each Aβ isoform.

Other aspects of the Aβ modeling calibration system enable users to interact with one or more graphical user interfaces to view and calibrate the optimized rate constant values, predicted fractional turnover rates, or in some embodiments, the kinetic model itself. The Aβ modeling calibration system 100 enables a user to select and manually or automatically adjust or modify one or more input values or rate constant values of the kinetic model.

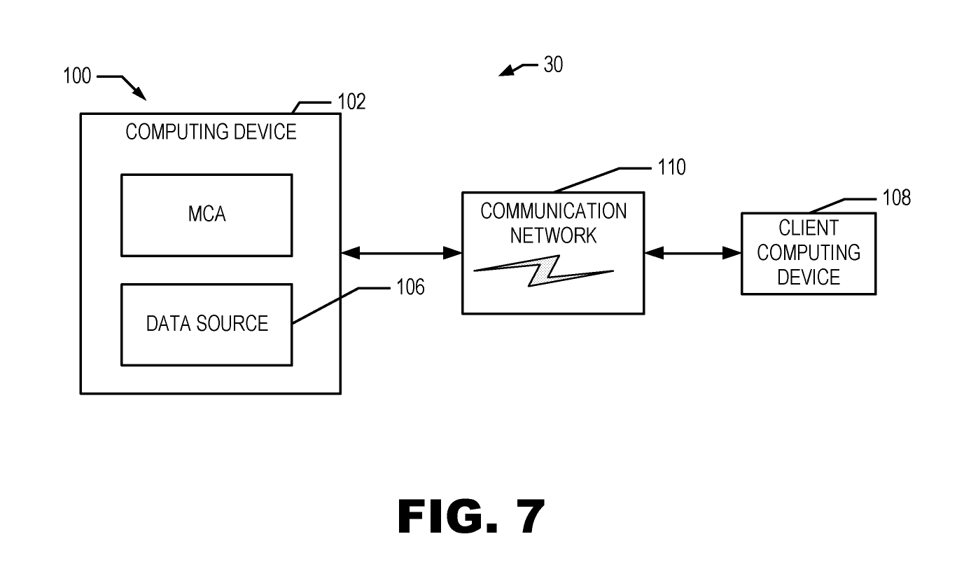

FIG. 7 is a block diagram of an exemplary computing environment 30 that includes an Aβ modeling calibration system (MCS) 100 in accordance with aspects of the disclosure. The MCS 100 includes a computing device 102 that includes an Aβ modeling application (MCA) 104 and a data source 106. The MCS 100 may be located on a single computing device 102. Alternately, the MCS 100 may be distributed across computing devices or located on a computing device configured as a server that communicates with one or more client computing devices (client) 108 via a communication network 110. Although the data source 106 is shown as being located on, at, or within the computing device 102, it is contemplated that the data source 106 can be located remotely from the computing device 102 in one or more other computing devices of the computing environment 30. For example, the data source 106 can be located on, at, or within a database of another computing device or system having at least one processor and volatile and/or non-volatile memory.

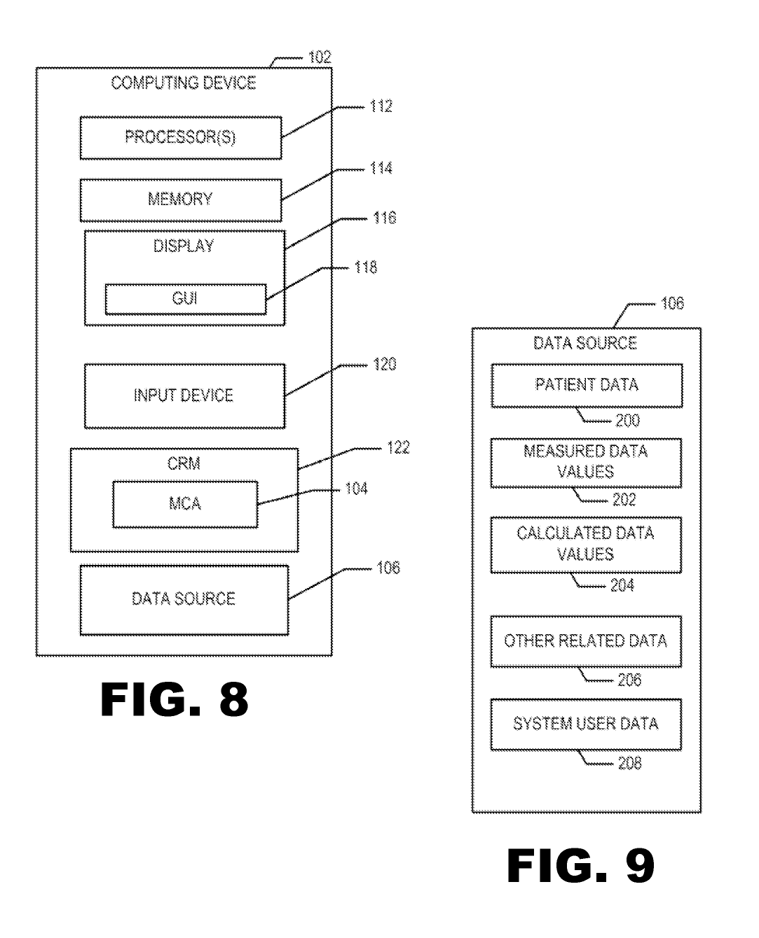

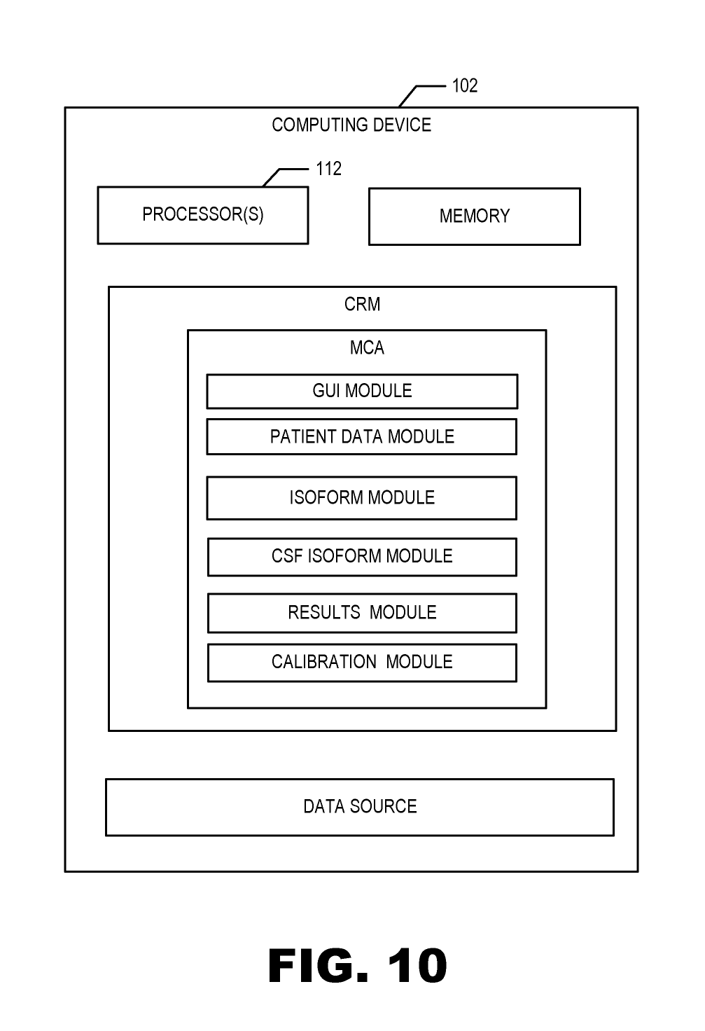

As shown in FIGS. 7, 8, and 10 the computing device 102 is a computer or processing device that includes one or more processors 112 and memory 114 to execute the MCA 104 to identify, determine, calibrate, and/or predict various values and constants of the kinetic model 20. The computing device 102 may also include a display device 116, such as a computer monitor, for displaying data and/or graphical user interfaces (GUIs) generated by a GUI module 300 of the MCA 104, as shown in FIG. 10. The computing device 102 may also include an input device 120, such as a keyboard or a pointing device (e.g., a mouse, trackball, pen, or touch screen) to enter data into or otherwise interact with various graphical user interfaces.

Each processing device 102 or 108 may also include a stand-alone or distributed version of the MCA application 104, to generate one or more graphical user interface(s) 120 on the display 114. The graphical user interface 120 enables a user of the processing devices 102 or 108 to view actual experimental data, predicted data, and other data manually input using the input device 116 or otherwise stored in the data source 106. The graphical user interface 120 also enables a user of the processing devices 102 or 108 to view and modify the stored data as well as any determined or predicted data values. According to another aspect, the graphical user interface 120 enables a user of the MCS system 100 to interact with various data entry forms to enter authentication data or other data, including but not limited to usernames, passwords or other user data, to access any restricted functionality of the MCS 100.

According to one aspect, the computing device 102 includes a computer-readable medium (“CRM”) 122, also referred to herein as a machine-readable medium, configured with the MCA 104. The CRM 122 includes instructions or modules that are executable by the processor(s) 112. The CRM 122 may include volatile media, nonvolatile media, removable media, non-removable media, and/or another available medium that can be accessed by the computing device 122. By way of example and not limitation, the CRM 122 comprises computer storage media and communication media. Computer storage media includes non-transient memory, volatile media, nonvolatile media, removable media, and/or non-removable media implemented in a method or technology for storage of information, such as computer readable instructions, data structures, program modules, or other data. Communication media may embody computer readable instructions, data structures, program modules, or other data and include an information delivery media or system

The data source 106 may be a database or other general repository of data including, but not limited to, MCS user data, patient data, model data, or any other data. The data source 106 or database may include memory and one or more processors or processing systems to receive, process, query, and transmit communications or requests to store and/or retrieve such data. In another aspect, the database may be a database server.

Similarly, the local or client computing device 108 may be a processing device similar to the processing device 102, one or more servers, personal computers, mobile computers, and other computing devices. In various aspects, the local computing devices 108 include one or more processors and volatile and/or non-volatile memory and may be configured to communicate over the communication network 112 via wireless and/or wireline communications.

The computing device 102 may be configured to receive data and/or communications from and/or transmit data and/or communications to a client 108 or other computing device, including a remote data source through the communication network 112. The communication network 112 can be can be the Internet, an intranet, and/or another wired and/or wireless communication network. In one aspect, the computing device 102, the client 108, and/or the data source 106 communicate data in packets, messages, or other communications using a protocol, such as a Hypertext Transfer Protocol (HTTP) or a Wireless Application Protocol (WAP). Other examples of communication protocols exist.

FIG. 9 depicts an exemplary embodiment of a data source 106 according to one aspect of the MCS 100. The data source 106 can be a local database or can be another server (not shown) that communicates with the computing device 102 via the communication network 212. According to one aspect, the data source 106 stores patient data 200, measured data values 202, predicted or determined data values 204, other related data 206, and MCS user data 208. Although the MCS 100 is depicted as including a single data source 106, it is contemplated that the MCS 100 may include multiple data sources in other aspects.

FIG. 10 depicts the computing device 102 with an exemplary embodiment of the MCA 104. As shown, the MCA 104 includes a number of modules 300-310 for performing a variety of functions, as explained more fully below. In various aspects, the functionality attributed to each module 300-310 may be performed by one or more other modules or a single module may perform some or all of the described functions.

Example embodiments of the methods and systems described herein may be implemented at least in part in electronic circuitry; in computer hardware executing firmware and/or software instructions, such as the MCA 104; and/or in combinations thereof. Example embodiments also may be implemented using a computer program product (e.g., a computer program tangibly or non-transitorily embodied in a machine-readable medium and including instructions for execution by, or to control the operation of, a data processing apparatus, such as, for example, one or more programmable processors or computers). A computer program may be written in any form of programming language, including compiled or interpreted languages, and may be deployed in any form, including as a stand-alone program or as a subroutine or other unit suitable for use in a computing environment. Also, a computer program can be deployed to be executed on one computer, or to be executed on multiple computers at one site or distributed across multiple sites and interconnected by a communication network.

Certain embodiments are described herein as including one or more modules and optionally, related sub-modules. Such modules are hardware-implemented, and thus include at least one tangible unit capable of performing certain operations and may be configured or arranged in a certain manner. For example, a hardware-implemented module may comprise dedicated circuitry that is permanently configured (e.g., as a special-purpose processor, such as a field-programmable gate array (FPGA) or an application-specific integrated circuit (ASIC)) to perform certain operations. A hardware-implemented module may also comprise programmable circuitry (e.g., as encompassed within a general-purpose processor or other programmable processor) that is temporarily configured by software or firmware to perform certain operations. In some example embodiments, one or more computer systems (e.g., a standalone system, a client and/or server computer system, or a peer-to-peer computer system) or one or more processors may be configured by software (e.g., an application or application portion) as a hardware-implemented module that operates to perform certain operations as described herein.

Accordingly, the term “hardware-implemented module” encompasses a tangible entity, be that an entity that is physically constructed, permanently configured (e.g., hardwired), or temporarily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure a processor, for example, to constitute a particular hardware-implemented module at one instance of time and to constitute a different hardware-implemented module at a different instance of time.

Hardware-implemented modules may provide information to, and/or receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and may store the output of that operation in a memory device to which it is communicatively coupled. A further hardware-implemented module may then, at a later time, access the memory device to retrieve and process the stored output. Hardware-implemented modules may also initiate communications with input or output devices.

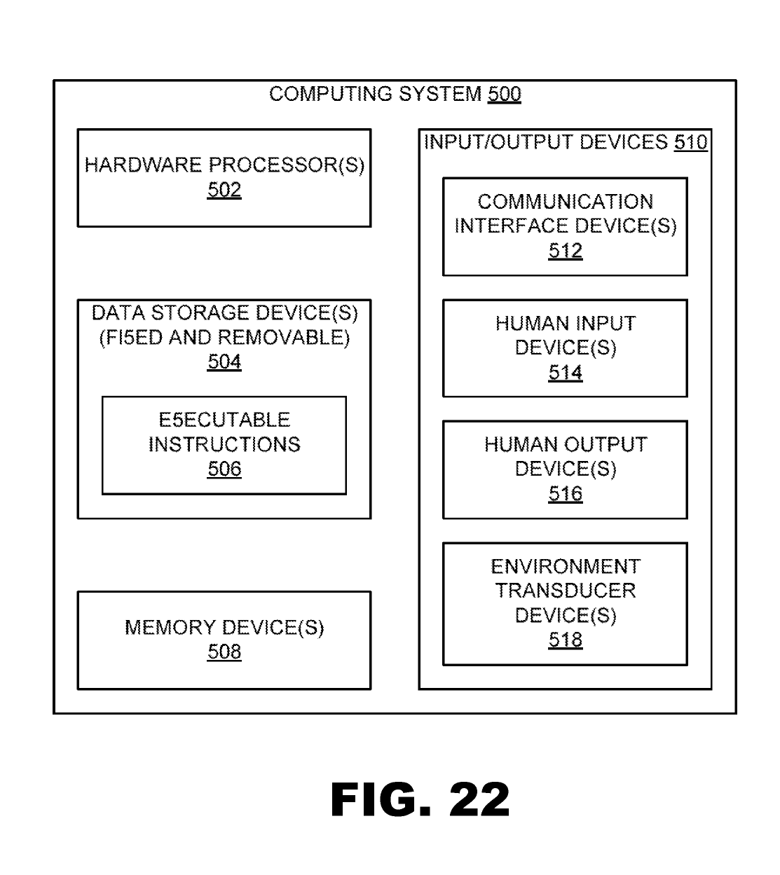

FIG. 22 is a block diagram of a machine in the example form of a computer system 500 within which instructions 506 for causing the machine to perform any one or more of the methodologies discussed herein may be executed by one or more hardware processors 502. In various embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. In some examples, the machine may be a desktop computer, a laptop computer, a tablet computer, a television receiver or set-top box (STB), a video streaming device, a smart television, a smartphone, a gaming system, a web appliance, a communication network node (e.g., a network router, switch, or bridge), a computing system embedded within another device or system (e.g., a household appliance), or any machine capable of executing instructions 506 (sequential or otherwise) that specify actions to be taken by that machine. Further, while only a single machine is illustrated, the term “machine” shall also be taken to include any collection of machines that individually or jointly execute a set (or multiple sets) of instructions 506 to perform any one or more of the methodologies discussed herein.

As depicted in FIG. 22, the example computing system 500 may include one or more hardware processors 502, one or more data storage devices 504, one or more memory devices 508, and/or one or more input/output devices 510. Each of these components may include one or more integrated circuits (ICs) (including, but not limited to, FPGAs, ASICs, and so on), as well as more discrete components, such as transistors, resistors, capacitors, inductors, transformers, and the like. Various ones of these components may communicate with one another by way of one or more communication buses, point-to-point communication paths, or other communication means not explicitly depicted in FIG. 22. Additionally, other devices or components, such as, for example, various peripheral controllers (e.g., an input/output controller, a memory controller, a data storage device controller, a graphics processing unit (GPU), and so on), a power supply, one or more ventilation fans, and an enclosure for encompassing the various components, may be included in the example computing system 500, but are not explicitly depicted in FIG. 22 or discussed further herein.

The at least one hardware processor 502 may include, for example, a central processing unit (CPU), a microprocessor, a microcontroller, and/or a digital signal processor (DSP). Further, one or more hardware processors 502 may include one or more execution cores capable of executing instructions and performing operations in parallel with each other.

The one or more data storage devices 504 may include any non-volatile data storage device capable of storing the executable instructions 506 and/or other data generated or employed within the example computing system 500. In some examples, the one or more data storage devices 504 may also include an operating system (OS) that manages the various components of the example computing system 500 and through which application programs or other software may be executed. Thus, in some embodiments, the executable instructions 506 may include instructions of both application programs and the operating system. Examples of the data storage devices 504 may include, but are not limited to, magnetic disk drives, optical disk drives, solid state drives (SSDs), flash drives, and so on, and may include either or both removable data storage media (e.g., Compact Disc Read-Only Memory (CD-ROM), Digital Versatile Disc Read-Only Memory (DVD-ROM), magneto-optical disks, flash drives, and so on) and non-removable data storage media (e.g., internal magnetic hard disks, SSDs, and so on).

The one or more memory devices 508 may include, in some examples, both volatile memory (such as, for example, dynamic random access memory (DRAM), static random access memory (SRAM), and so on), and non-volatile memory (e.g., read-only memory (ROM), flash memory, and the like). In one embodiment, a ROM may be utilized to store a basic input/output system (BIOS) to facilitate communication between an operating system and the various components of the example computing system 500. In some examples, DRAM and/or other rewritable memory devices may be employed to store portions of the executable instructions 506, as well as data accessed via the executable instructions 506, at least on a temporary basis. In some examples, one or more of the memory devices 508 may be located within the same integrated circuits as the one or more hardware processors 502 to facilitate more rapid access to the executable instructions 506 and/or data stored therein.

The one or more data storage devices 504 and/or the one or more memory devices 508 may be referred to as one or more machine-readable media, which may include a single medium or multiple media (e.g., a centralized or distributed database, and/or associated caches and servers) that store the one or more executable instructions 506 or data structures. The term “machine-readable medium” shall also be taken to include any tangible medium that is capable of storing, encoding, or carrying instructions 506 for execution by the machine and that cause the machine to perform any one or more of the methodologies of the present invention, or that is capable of storing, encoding, or carrying data structures utilized by or associated with such instructions 506.

Machine-readable media may also include transitory and non-transitory communication media. Communication media includes computer-readable instructions, data structures, hardware-implemented modules, program modules or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof.

The input/output devices 510 may include one or more communication interface devices 512, human input devices 514, human output devices 516, and environment transducer devices 518. The one or more communication interface devices 512 may be configured to transmit and/or receive information between the example computing system 500 and other machines or devices by way of one or more wired or wireless communication networks or connections. The information may include data that is provided as input to, or generated as output from, the example computing device 500, and/or may include at least a portion of the executable instructions 506. Examples of such network or connections may include, but are not limited to, Universal Serial Bus (USB), Ethernet, Wi-Fi®, Bluetooth®, Near Field Communication (NFC), Long-Term Evolution (LTE), and so on. One or more such communication interface devices 510 may be utilized to communicate one or more other machines, either directly over a point-to-point communication path, over a wide area network (WAN) (e.g., the Internet), over a local area network (WAN), over a cellular (e.g., third generation (3G) or fourth generation (4G)) network, or over another communication means. Further, one or more of one of wireless communication interface devices 512, as well as one or more environment transducer devices 518 described below, may employ an antenna for electromagnetic signal transmission and/or reception. In some examples, an antenna may be employed to receive Global Positioning System (GPS) data to facilitate determination of a location of the machine or another device.

In some embodiments, the one or more human input devices 514 may convert a human-generated signal, such as, for example, human voice, physical movement, physical touch or pressure, and the like, into electrical signals as input data for the example computing system 500. The human input devices 514 may include, for example, a keyboard, a mouse, a joystick, a camera, a microphone, a touch-sensitive display screen (“touchscreen”), a positional sensor, an orientation sensor, a gravitational sensor, an inertial sensor, an accelerometer, and/or the like.

The human output devices 516 may convert electrical signals into signals that may be sensed as output by a human, such as sound, light, and/or touch. The human output devices 516 may include, for example, a display monitor or touchscreen, a speaker, a tactile and/or haptic output device, and/or so on.

The one or more environment transducer devices 518 may include a device that converts one form of energy or signal into another, such as from an electrical signal generated within the example computing system 500 to another type of signal, and/or vice-versa. Further, the transducers 518 may be incorporated within the computing system 500, as illustrated in FIG. 22, or may be coupled thereto in a wired or wireless manner. In some embodiments, one or more environment transducer devices 518 may sense characteristics or aspects of an environment local to or remote from the example computing device 500, such as, for example, light, sound, temperature, pressure, magnetic field, electric field, chemical properties, physical movement, orientation, acceleration, gravity, and so on. Further, in some embodiments, one or more environment transducer devices 518 may generate signals to impose some effect on the environment either local to or remote from the example computing device 500, such as, for example, physical movement of some object (e.g., a mechanical actuator), heating or cooling of a substance, adding a chemical substance to a substance, and so on.

### III. Methods of Using the Model of In Vivo Metabolism of Aβ

The present disclosure provides methods of using a model of the in vivo metabolism of a CNS biomolecule or Aβ. The model may be used to calculate metabolic parameters, such as the synthesis and clearance rates within the CNS, in one aspect. In an aspect, the kinetic model may be used to identify the disease state of a patient by comparing an index calculated from model parameters to a pre-selected threshold. In another aspect, the kinetic model may be used to predict the metabolism and/or concentration of Aβ or its various isoforms in a patient in vivo. In an aspect, the model may be used to create a curve fit for each Aβ isoform time course in a patient. In yet another aspect, the model may be used to identify sensitive pathway components to help design drugs or understand a CNS disease. In even another aspect, the model may be used to investigate changes in the kinetics of the isoforms that may be induced by investigational drugs. In one aspect, the model may be used to characterize Aβ in various patients.

### (a) Identifying a Patient's Disease State

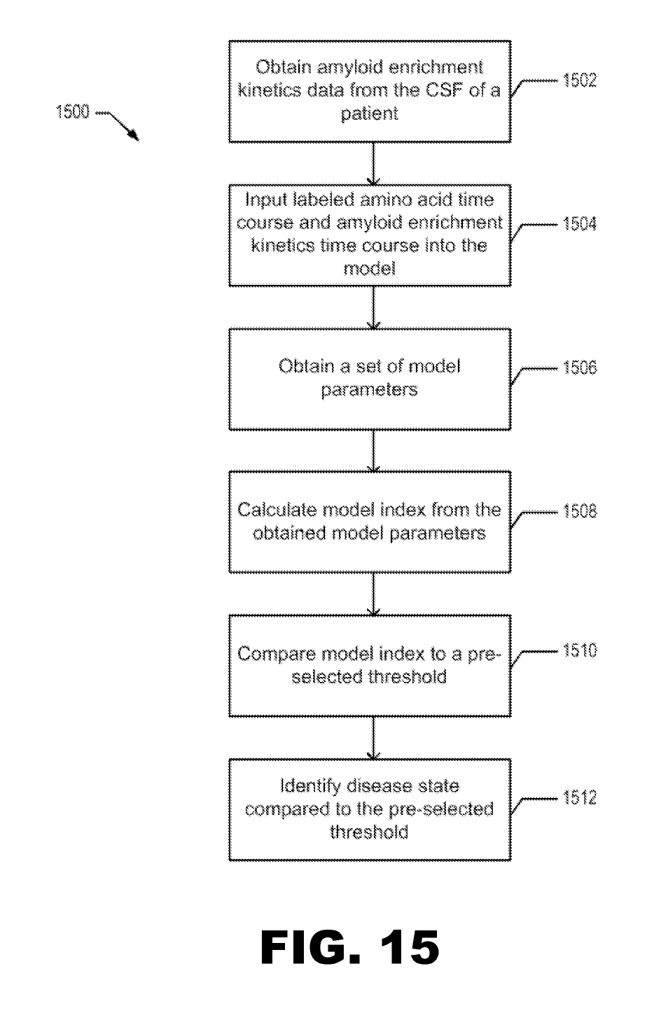

FIG. 15 is an illustration of a method of using the kinetic model to identify the disease state of a patient. The method of using the model 1500 may include obtaining Aβ enrichment kinetics data from the CSF of the patient as depicted in step 1502; inputting the time course data from a labeled moiety, the Aβ42 enrichment kinetics in the CSF, and at least one other Aβ isoform enrichment kinetics in the CSF into the kinetic model as depicted in step 1504; obtaining a set of model parameters from the kinetic model as depicted in step 1506; calculating a model index comprising a mathematical calculation with at least one model parameter from the kinetic model as depicted in step 1508; comparing the model index to a pre-selected threshold as depicted in step 1510; and identifying the disease state of the patient as depicted in step 1512.

In an aspect, the kinetic model may represent enrichment kinetics of Aβ42 and at least one other Aβ isoform. In this aspect, the labeled moiety may be labeled plasma leucine. One of skill in the art will appreciate that other Aβ isoforms may include, but are not limited to, Aβ37, Aβ38, Aβ39, Aβ40, Aβ41, total Aβ, as well as enzymatic digestion products thereof. The Aβ enrichment kinetics data from a patient may be obtained by the SILK method and may include time course data for Aβ42, Aβ40, and/or Aβ38 in the CSF. In an aspect, the data input into the kinetic model may include the time course of Aβ42 in the CSF and the time course of Aβ40 in the CSF. In another aspect, the data input into the kinetic model may include the time course of Aβ42 in the CSF and the time course of Aβ38 in the CSF. In yet another aspect, the data input into the kinetic model may include the time course of Aβ42 in the CSF, the time course of Aβ40 in the CSF, and the time course of Aβ38 in the CSF. In an aspect, the time course data of labeled plasma leucine may be input into the kinetic model.

The input of the data into the kinetic model may create a set of model parameters for that patient. The model parameters obtained from the kinetic model may include, but are not limited to, the concentration of Aβ isoforms, rates of transfer (e.g. k_{APP}, k_{C99}, k_{Ab42}, k_{Ab40}, k_{Ab38}), rates of irreversible loss (e.g. v_{APP}, v_{C99}, v₄₂, v₄₀, v₃₈), rates of exchange (e.g. kₑₓ₄₂, kᵣₑₜ), rates of delay (e.g. k_{delay}), or any parameter that may be used in the kinetic model. The model index may be calculated using at least one model parameter. The model index may be calculated using any mathematical operator with the at least one model parameters, including but not limited to multiplication, division, addition, subtraction, logarithm, or any other mathematical operator. In an aspect, the model index may be calculated using the model parameters for the rate of irreversible loss of Aβ42 and the rate of transfer of Aβ42. In one aspect, the model index may be calculated using the calculation shown in Eqn. (I) below:

(10λk_{Ab42})+v₄₂ Eqn. (I)

In another aspect, the model index may be calculated using a model parameter of Aβ42 and the same model parameter of another Aβ isoform (e.g. Aβ37, Aβ38, Aβ39, Aβ40, Aβ41, total Aβ, or other Aβ isoforms known in the art). By way of non-limiting examples, a model index may be calculated using a calculation shown in Eqn. (II) to Eqn. (XI) below:

Aβ42 peak time/Aβ40 peak time Eqn. (II)

Aβ42 peak time/Aβ39 peak time Eqn. (III)

Aβ42 peak time/Aβ38 peak time Eqn. (IV)

Aβ42 peak time/Aβ37 peak time Eqn. (V)

Aβ42 peak time/total Aβ peak time Eqn. (VI)

Aβ42 FTR/Aβ40 FTR Eqn. (VII)

Aβ42 FTR/Aβ39 FTR Eqn. (VIII)

Aβ42 FTR/Aβ38 FTR Eqn. (IX)

Aβ42 FTR/Aβ37 FTR Eqn. (X)

Aβ42 FTR/total AβFTR Eqn. (XI)

Other aspects describing alternative model indices are described herein below in the Examples.

A pre-selected threshold may be calculated in the same manner as the model index using the model parameters of other patients or an average of model parameters from other patients with a known disease state. The method of using the kinetic model to identify the disease state of a patient may include identifying Alzheimer's disease in the patient. In an aspect, the disease state may be identified as Alzheimer's if the model index is above a pre-selected threshold for Alzheimer's. In another aspect, the severity of the disease state may be identified by comparing the model index to a pre-selected correlation of the disease state. In one aspect, the correlation of the disease state may be identified by PIB imaging.

### (b) Producing a Curve Fit for Measured Data

The kinetic model may be used to create a curve fit for each Aβ isoform time course in a patient. In an aspect, limited data from a patient may be input into the model and the model may produce a curve fit for each Aβ isoform time course from the data provided. The curve fit may be used to predict unknown metabolism of Aβ and project to a later time course.

### (c) Predicting Metabolism or Concentration

The kinetic model may be used to predict the metabolism and/or concentration of Aβ in a patient. In an aspect, a database of parameters, as described herein above, may be used within the model to predict the metabolism of a Aβ isoform in a patient by using the set of parameters from the database that most closely match the genotype or phenotype of the patient. In another aspect, the model may be used to predict the concentration of different Aβ isoforms at different locations within the body and/or at different time points. In another aspect, the model may be used to calculate the metabolic parameter within the model.

### (d) Identifying a Sensitive Pathway

The kinetic model may be used to identify sensitive pathway components to help design drugs or understand a CNS disease. In an aspect, compartments may be added or subtracted to observe the effect of the concentrations and rate constants of the Aβ isoforms. In one aspect, the addition or subtraction of compartments may indicate sensitive areas within the pathway and may indicate areas for potential drug action. In another aspect, the rate constants within the model may be increased or decreased to observe the effect of the concentrations and other rate constants of the Aβ isoforms. In one aspect, the adjustment of the rate constants may indicate sensitive areas within the pathway and may indicate areas for potential drug action.

### (e) Simulating the Action of a Drug

The model may be manipulated to simulate the action of a drug within the CNS. In an aspect, the model may be used to investigate changes in the kinetics of the Aβ isoforms that may be induced by investigational drugs. In one aspect, the model parameters may be adjusted to best represent the effect of a drug on a patient in vivo. In another aspect, the model may be used to predict CSF concentrations of at least one Aβ isoform CSF concentration.

### (f) Characterizing Aβ

The model may be used to characterize Aβ kinetics in various patients. In an aspect, the parameters in the database may be used to predict the kinetics of Aβ in other patients. In an aspect, a non-carrier patient may be modeled using the parameters in the database for a non-carrier without the need to measure the concentration of the Aβ isoforms in the CSF. In an aspect, a MC PIB− patient may be modeled using the parameters in the database for MC PIB− without the need to measure the concentration of the Aβ isoforms in the CSF. In an aspect, a MC PIB+ patient may be modeled using the parameters in the database for MC PIB+ without the need to measure the concentration of the Aβ isoforms in the CSF.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Mutation and Amyloid Deposition was Modeled by Differential Aβ Isoform Kinetics

The following experiment assessed the development of a model of Aβ trafficking in vivo using data from SILK studies.

The model consisted of the following structure and parameters. The rate of production of APP was governed by the product of the zero-order rate constant k_{APP} and the fraction of isotope-labeled leucine. The units of ‘concentrations’ were ng per mL of CSF, thus not accounting explicitly for the volume of the brain compartment. The APP degradation product C99 was produced at a rate governed by the product of the rate constant k_{C99} and the concentration of APP. C99 was further processed into the three Aβ peptides, Aβ38, Aβ40 and Aβ42 at rates governed by the product of the concentration of C99 and the rate constants k_{Aβ38}, k_{Aβ40} and k_{Aβ42}, respectively. C99 may also be irreversibly degraded to produce other products, governed by the product of the rate constant \dot{V}_{C99} and the C99 concentration. All irreversible clearance processes that occur within the brain (degradation, transport to the vasculature and deposition into plaques) may be described by product of the rate constants \dot{V}₃₈, \dot{V}₄₀ or \dot{V}₄₂ multiplied by the soluble brain concentration of Aβ38, Aβ40 and Aβ42, respectively. Transport of the CSF to the lumbar space may be modeled as three CSF delay compartments with equal rate constants for entry and exit (k_{delay}). The concentration of predicted labeled Aβ peptide in the third delay compartment was compared to the total measured concentration of Aβ peptide in the CSF to compute a predicted fractional labeling. The parameters were optimized against the measured fractional labeling of the Aβ peptide.

In vivo SILK studies were performed in participants with ADAD mutations and sibling non-carrier controls. The Aβ kinetic parameters were compared by the presence of a PSEN mutation and insoluble amyloid deposition as measured by PiB-PET.

SILK studies were performed in 23 patients (11 with mutations in PSEN1 or PSEN2, 12 non-mutation carrier sibling controls) using a 9-h primed constant infusion of ¹³C₆ leucine. Seven mutation carriers had evidence of plaques by PiB PET; the remaining mutation carriers and all non-carriers were PiB negative. Four mutation carriers were cognitively symptomatic, all other participants were cognitively normal. CSF Aβ38, Aβ40, and Aβ42 concentrations and isotopic enrichments were measured at hourly intervals over a 36 h period.

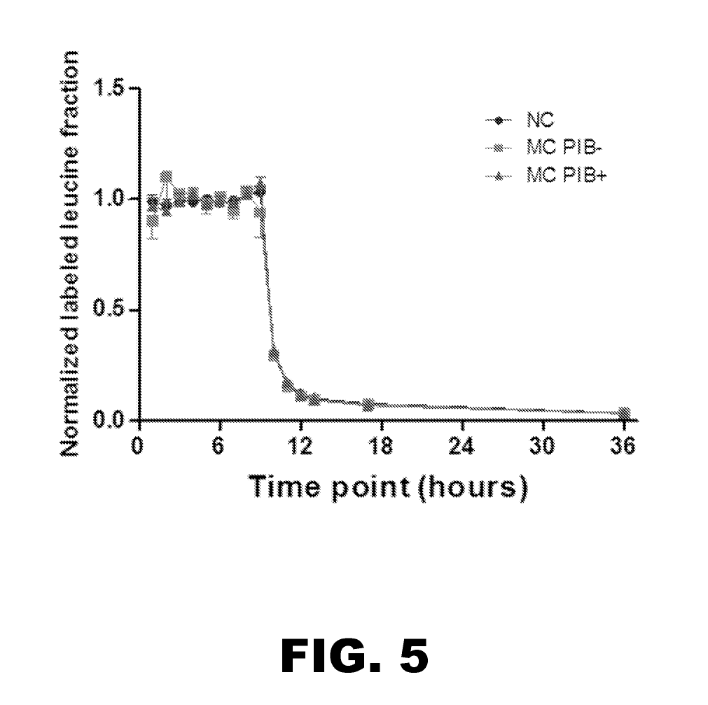

During the ¹³C₆-leucine infusion, plasma leucine enrichment approximated a constant plateau and then rapidly decreased after the infusion was stopped (FIG. 5). The ¹³C₆-leucine isotopic enrichments of Aβ38, Aβ40, and Aβ42 were compared between mutation carriers, with or without amyloidosis, and non-mutation carriers to address the relationship between Aβ isoform metabolic kinetics, mutation status, and amyloid deposition (PIB+ indicates fibrillar amyloid deposition as measured by PET with Pittsburgh Compound B).

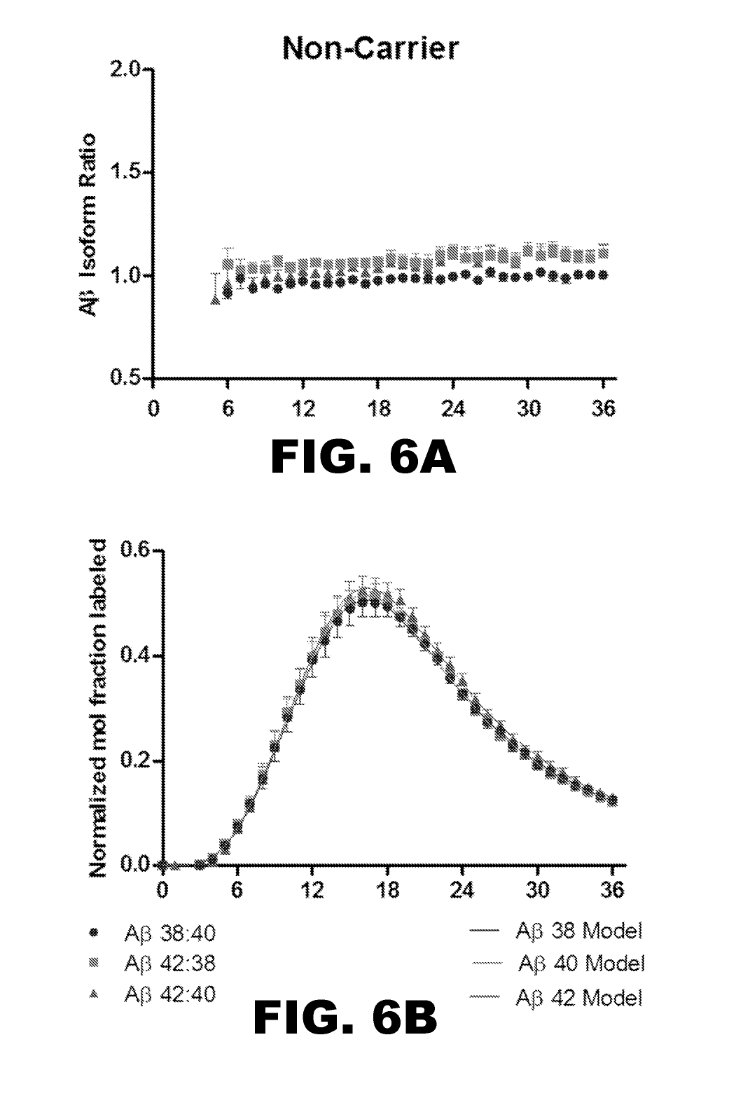

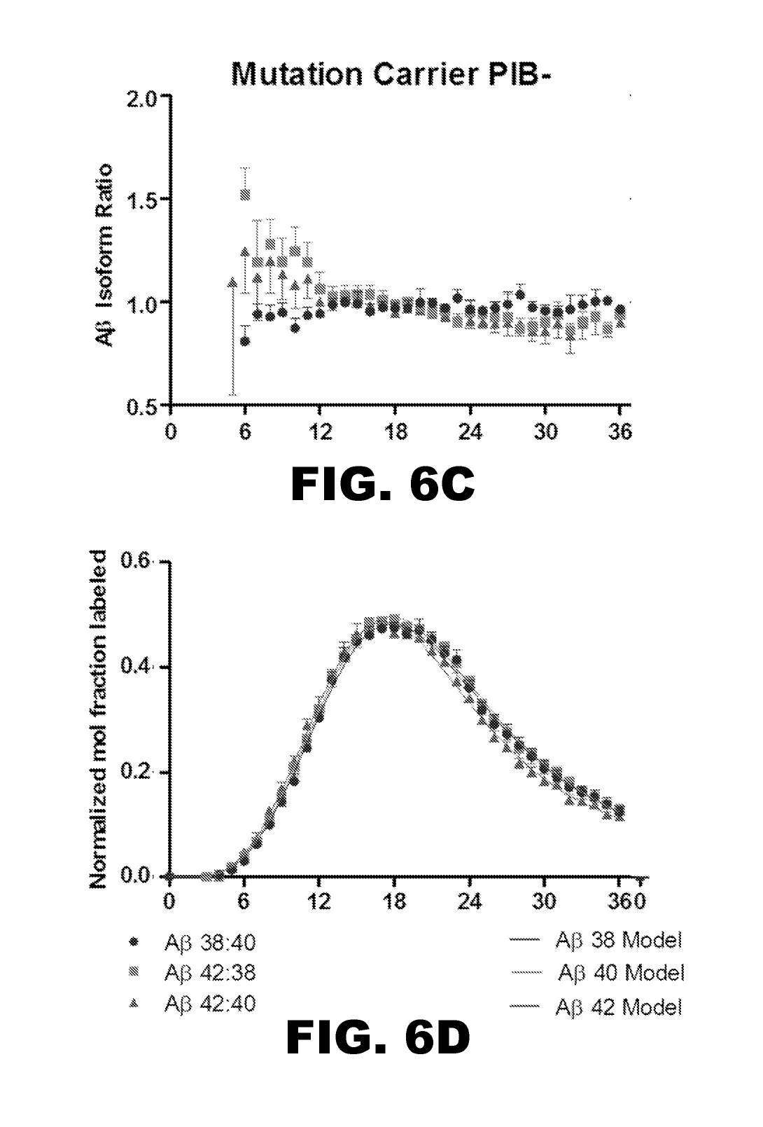

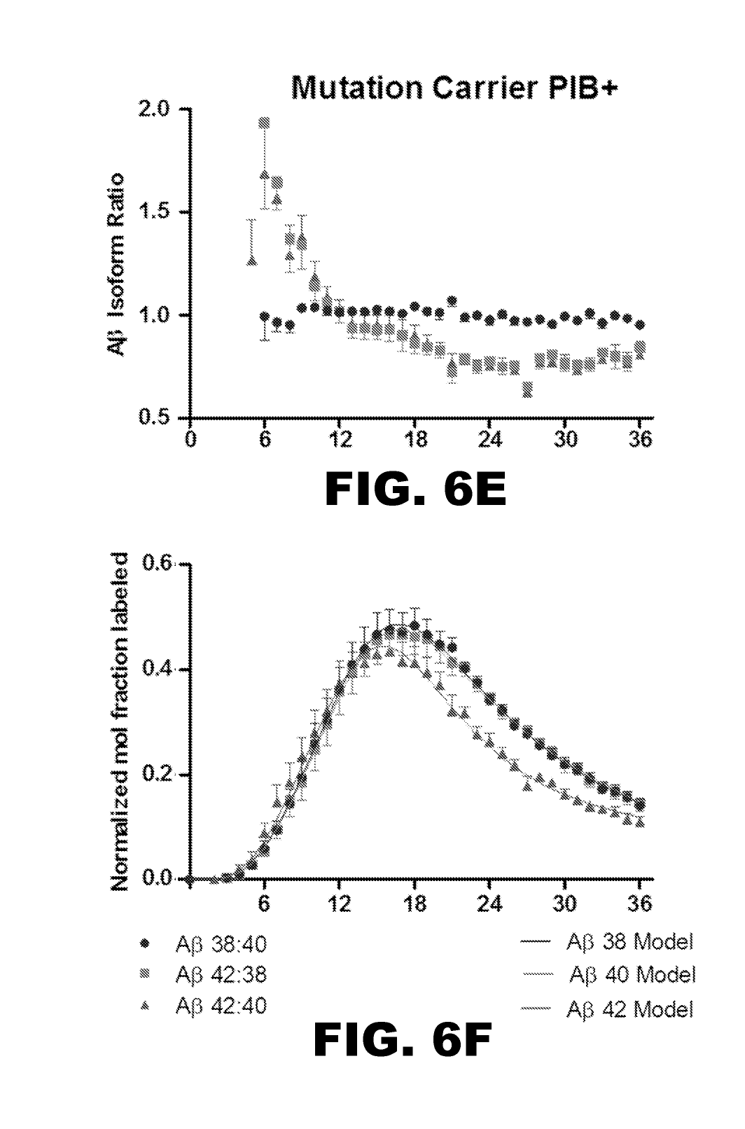

To compare Aβ isoform kinetics, ratios of labeled Aβ isoform enrichments in the CSF were plotted so that a ratio of one indicates the same isotopic enrichment and kinetics between Aβ isoforms. The Aβ38:Aβ40 labeling ratio was approximately constant at one over time in all patient groups (FIG. 6A), indicating similar kinetics between Aβ38 and Aβ40. Similarly, the Aβ42:40 and Aβ42:38 labeling ratios were nearly constant at one over time in non-carriers. However, in both PIB− and PIB+ mutation carriers, the Aβ42:40 and Aβ42:38 labeling ratios were elevated during early time points and decreased in later time points (FIG. 6A). The Aβ isoform enrichment mismatch was more pronounced in participants with amyloid deposition (PIB+), caused by an earlier and lower Aβ42 peak with a flatter terminal tail compared to Aβ38 and Aβ40 (FIG. 6B). The time to reach peak ¹³C-labeling in each Aβ isoform was measured for each patient. The Aβ38:Aβ40 peak time ratio was not different between mutation carrier and non-carrier groups (1.01±0.01 vs. 1.00±0.01, respectively). In contrast, Aβ42 peaked at the same time as Aβ40 in the non-carrier group (Aβ42:Aβ40 peak time ratio=1.01±0.03), whereas Aβ42 peaked significantly earlier than Aβ40 in the mutation group (peak time ratio=0.93±0.05, p=0.015 mutation effect, p<0.001 for PIB score).

A comprehensive compartmental model similar to the models described previously herein was developed to quantify steady state Aβ isoform kinetic parameters. The model incorporated the plasma leucine and Aβ enrichment time course profiles and the CSF Aβ isoform concentrations for each patient (schematic diagram in FIG. 3). FIG. 4 is a detailed figure of the model. FIG. 6B shows curve fits from the model for average Aβ isoform time course profiles as enrichments normalized to plasma leucine. A reversible exchange compartment was incorporated to model the sigmoidal decay of many labeling curves, especially Aβ42 in PIB+ participants. The model included an irreversible loss of each soluble Aβ isoform that was not recovered in CSF. The rate constants for transfer between compartments in the model were calibrated using measured values for each patient. Mean values for each parameter are summarized in Table 1 below.

TABLE 1

| | | Mutation- | Mutation- |
|---|---|---|---|
| Parameter | Non-carriers | carrier PIB− | carrier PIB+ |
| k_{APP} | 1,171 ± 227 | 1,304 ± 602 | 1,291 ± 324 |
| k_{C99} | 0.666 ± 0.112 | 0.553 ± 0.083 | 0.695 ± 0.096 |
| k_{Aβ38} | 0.062 ± 0.010 | 0.055 ± 0.016 | 0.059 ± 0.008 |
| k_{Aβ40} | 0.238 ± 0.041 | 0.187 ± 0.023 | 0.247 ± 0.037 |
| k_{Aβ42} | 0.033 ± 0.006 | 0.034 ± 0.007 | 0.041 ± 0.006 |
| v_{C99} | 0.333 ± 0.056 | 0.276 ± 0.041 | 0.347 ± 0.048 |
| v₃₈ | 0.069 ± 0.023 | 0.075 ± 0.027 | 0.054 ± 0.015 |
| v₄₀ | 0.074 ± 0.023 | 0.082 ± 0.037 | 0.050 ± 0.013 |
| v₄₂ | 0.064 ± 0.014 | 0.126 ± 0.072 | 0.120 ± 0.037 |
| k_{CSF} | 0.074 ± 0.023 | 0.082 ± 0.037 | 0.050 ± 0.013 |
| kₑₓ₃₈ | 0.020 ± 0.038 | 0.000 ± 0.000 | 0.000 ± 0.000 |
| kₑₓ₄₀ | 0.016 ± 0.032 | 0.009 ± 0.018 | 0.000 ± 0.000 |
| kₑₓ₄₂ | 0.010 ± 0.021 | 0.041 ± 0.045 | 0.120 ± 0.107 |
| kᵣₑₜ | 0.1 | 0.1 | 0.1 |
| k_{delay} | 0.666 ± 0.112 | 0.553 ± 0.083 | 0.695 ± 0.096 |
| SF₃₈ | 0.937 ± 0.066 | 0.885 ± 0.063 | 0.979 ± 0.092 |
| SF₄₀ | 0.933 ± 0.043 | 0.916 ± 0.078 | 0.977 ± 0.130 |
| SF₄₂ | 0.972 ± 0.102 | 0.879 ± 0.021 | 0.912 ± 0.151 |

The results of this experiment demonstrated that biological mechanisms and patient data that account for Aβ isoform-specific differences may be used to develop a model of Aβ isoform kinetics and the model may provide insights into the metabolic kinetics of Aβ peptides by both mutation and amyloid deposition status.

### Example 2

### An Exchange Process was Required to Fit Aβ Kinetic Curves

To demonstrate the ability of the model to account for exchange with unlabeled Aβ peptides, the following experiment was conducted.

Using the model developed in Example 1, additional compartments were added to further develop the model. To optimally fit the shape and peak magnitude of Aβ isoform enrichment time courses, a compartment was required to model reversible exchange of newly synthesized labeled Aβ peptides with a pre-existing pool of unlabeled Aβ, as shown in FIG. 3. The exchange process was of minimal magnitude in non-mutation carriers, in which only about 10% of the flux of newly synthesized Aβ38, 40 or 42 underwent exchange (Table 2). The percent of Aβ38 and Aβ40 that underwent exchange was not significantly different between mutation carriers and non-carriers. However, the exchange for Aβ42 was significantly greater in carriers compared to the non-carriers (51±58% vs. 6±12% of flux, respectively, p=0.004 for mutation effect, p=0.001 for PIB status) (Table 2). The exchange process for Aβ42, combined with the faster turnover rate of Aβ42, provided an excellent fit to the entire shape of the Aβ42 enrichment time course in all groups including mutation carriers with amyloid deposition (mean R² for all participants of 0.994, 0.995, and 0.987 for Aβ38, Aβ40 and Aβ42, respectively).

TABLE 2

| | Non- | Mutation+ | | |
|---|---|---|---|---|
| | carriers | carriers | | |
| | (n = 13) | (n = 13) | p-values^{b} | |
| Production rate, ng/h (e.g. C99 | | | Mutation | |
| pool size × k_{Aβ42}) | | | status | PIB MCBP |
| Aβ38 | 106[41] | 111[50] | 0.603 | 0.571 |
| Aβ40 | 418 ± 83 | 452 ± 138 | 0.621 | 0.901 |
| Aβ42 | 57[19] | 67[35] | 0.038 | 0.769 |
| Aβ38:Aβ40 | 0.267[0.021] | 0.252[0.052] | 0.692 | 0.179 |
| ratio | | | | |
| Aβ42:Aβ40 | 0.140 ± 0.011 | 0.174 ± 0.020 | 9510⁻⁵ | 0.312 |
| ratio | | | | |
| Percentage of flux going | | | Mutation | |
| to exchange (%)* | | | status | PIB status |
| Aβ38 | 9.8 ± 16.6 | 0^{a} | 0.19 | 0.376 |
| Aβ40 | 7.8 ± 13.9 | 1.2 ± 4.1 | 0.316 | 0.249 |
| Aβ42 | 5.8 ± 11.5 | 50.8 ± 57.6 | 0.004 | 0.001 |
| Permanent loss of soluble Aβ to all | | | | |
| fates (fractional turnover rate, FTR) | | | Mutation | |
| (pools/h) (e.g. v₄₂ + k_{CSF}) | | | status | PIB MCBP |
| Aβ38 | 0.144 ± 0.046 | 0.124 ± 0.049 | 0.802 | 0.054 |
| Aβ40 | 0.156[0.055] | 0.109[0.035] | 0.99 | 0.024 |
| Aβ42 | 0.147[0.049] | 0.198[0.086] | 0.065 | 0.548 |
| Aβ38:40 | 0.964 ± 0.038 | 1.013 ± 0.047 | 0.157 | 0.115 |
| ratio | | | | |
| Aβ42:40 | 0.942 ± 0.080 | 1.553 ± 0.382 | 0.0016 | 0.0003 |
| ratio | | | | |
| CSF concentration | | | Mutation | |
| by IP-MS (ng/mL) | | | status | PIB MCBP |
| Aβ38 | 2.05[0.69] | 1.82[1.00] | 0.296 | 0.105 |
| Aβ40 | 7.15 ± 1.80 | 7.79 ± 1.89 | 0.199 | 0.272 |
| Aβ42 | 1.01[0.39] | 0.80[0.52] | 0.537 | 0.007 |
| Aβ38:Aβ40 | 0.272 ± 0.014 | 0.256 ± 0.053 | 0.803 | 0.068 |
| ratio | | | | |
| Aβ42:Aβ40 | 0.149 ± 0.013 | 0.121 ± 0.042 | 0.72 | 0.003 |
| ratio | | | | |

The results of this experiment demonstrated that a compartment for the exchange of labeled Aβ peptides with unlabeled peptides was necessary to model the exchange of Aβ42, particularly in mutation carrier groups.

### Example 3

### Higher Irreversible Loss of Aβ42 in Amyloid Deposition was Assessed

To assess the ability of the model to account for irreversible loss, the following experiment was conducted. Using the model of Examples 1 and 2, additional compartments were added to further develop the model. The fractional turnover rate (FTR, pools/h) of soluble Aβ is the rate constant for permanent loss of soluble Aβ and is kinetically distinct from reversible exchange. The physiology of the system suggests that FTR includes irreversible losses to the CSF or bloodstream, degradation, and deposition into amyloid plaques, as illustrated in FIG. 2. The model was adjusted to include fractional turnover rates, or rate of irreversible loss, for the various isoforms and each type of patient. The Aβ40 FTR was significantly slower in PIB+ compared to PIB− participants (p=0.024 for PIB effect) and trended towards significance for Aβ38 (p=0.054 for PIB effect), but neither was affected by mutation status (Table 2). The decreased turnover rate was thus associated with the presence of PIB− detectable amyloid plaques. In contrast, Aβ42 FTR trended towards an increase in mutation carriers (p=0.065 for mutation effect) independent of amyloid load (Table 2). The Aβ38:Aβ40 FTR ratio was not significantly different between non-carrier and mutation carrier groups, but the Aβ42:Aβ40 FTR ratio was 65% higher in mutation carriers (p<0.002 for both mutation status and PIB score) (Table 2).

The measured concentration of CSF Aβ isoforms were compared by mutation status and PIB score (Table 2). The Aβ42 CSF concentration and the Aβ42:Aβ40 CSF concentration ratio were significantly reduced in association with amyloid deposition (p=0.003 for PIB score; not significant by mutation status), whereas there were no differences between groups for the CSF Aβ38, Aβ40, or Aβ38:Aβ40 concentration ratio. The results of this experiment confirmed that the model may be adapted to account for irreversible loss of each isoform.

### Example 4

### One-Dimensional Flow of Aβ in the Brain was Modeled

To assess the feasibility of using a one dimensional flow model to describe isotope labeling kinetics, the following experiments were conducted.

A one-dimensional flow of Aβ from the brain's interstitial fluid (ISF) to the CSF, was incorporated into a model similar to the mode described in Examples 1 and 2. The model is summarized in the schematic in FIG. 12 incorporated the following changes in structure and parameters. The APP compartment was divided into an immature APP and a mature APP compartment. The rate of production of iAPP was governed by the product of the zero-order rate constant k_{iAPP} and the fraction of isotope-labeled leucine. The immature APP was assumed to be processed (glycosylated) to produce mature APP. The rate of production of mAPP was governed by the product of the first-order rate constant k_{mAPP} and the ‘concentration’ of iAPP. The APP degradation product C99 was produced at a rate governed by the product of the rate constant k_{C99} and the concentration of mAPP.

All three peptides flow with the brain interstitial fluid and any Aβ peptide that is transported to the surface of the brain without being cleared then becomes part of the CSF. Aβ42 may also enter a reversible exchange compartment, which was previously found to be more substantially exchanged than Aβ38 or Aβ40. Aβ42 within the exchange compartment is not subject to flow. The soluble Aβ42 concentration in the brain does not include the amount of Aβ42 within the exchange compartment. Transport of the CSF to the lumbar space was modeled as two delay compartments with equal rate constants for entry and exit (k_{delay}).

A length from ventricle to brain surface was taken as 3 cm or 7 cm. The 7 cm value had been adopted in a previous model of ISF flow, but the 3 cm was considered more realistic. The one-dimensional flow model is further summarized in FIG. 13. The one-dimensional flow model was integrated with the compartmental model shown in FIG. 12 to model in vivo Aβ labeling kinetics.

FIG. 13 illustrates the brain, represented by the box, with the ventricles on the left and the brain surface on the right. C99 was represented as being bound to the brain, uniformly distributed within the brain compartment, along the one-dimensional distance from the ventricle, x. C99 was not subject to ISF flow. Each location has a source of C99 that produces Aβ. Upon enzymatic cleavage of the C99, the Aβ peptides are released and transported along with the flowing ISF. The Aβ released from each location joins in the ISF flow. The Aβ peptides may be cleared and/or degraded in the flow (decreasing concentrations are depicted as narrowing lines in FIG. 13), and any Aβ peptide that reaches the surface of the brain at x=1 then becomes part of the CSF.

To develop the one-dimensional flow model from the ventricle to the surface of the brain, the continuity equation was used as shown in Eqn. (2-1) and the one dimensional momentum balance was used as shown in Eqn. (2-2):

$$\begin{matrix}\frac{d {v}_{x}}{d x} = {F}_{V} & Eqn . ( 2 \text{-} 1 ) \\ ρ ( \frac{\partial {v}_{x}}{\partial t} + {v}_{x} \frac{\partial {v}_{x}}{\partial x} ) = - \frac{\partial {P}_{i}}{\partial x} + μ \frac{{\partial }^{2} {v}_{x}}{\partial {x}^{2}} - \frac{μ}{κ} {v}_{x} , & Eqn . ( 2 \text{-} 2 )\end{matrix}$$

where Fᵥ is the rate or production of fluid by the capillaries per unit volume of fluid, v is the velocity of the fluid, and x is the normalized distance from the ventricles. Eqn. (I) expresses the change in velocity of the fluid as due solely to the introduction of new fluid from the capillaries. As more fluid is added, the velocity of the fluid must increase due to the incompressibility of water.

The introduction of fluid from the capillaries due to higher pressure in the vasculature is assumed to follow Starling's Law, as shown in Eqn. (2-3):

Fᵥ=Lₚ(S/V)[ρ_{vascular}−ρᵢ−(π_{vascular}−πᵢ] Eqn. (2-3)

A non-dimensionalized continuity equation for generality becomes:

$$\frac{d {\overline{v}}_{x}}{d \overline{x}} = \frac{L}{{v}_{s}} {L}_{P} ( S \text{/} V ) [ {P}_{vascular} - {P}_{i} - σ ( {π}_{vascular} - {π}_{i} ) ] = {\overline{F}}_{V} , \qquad Eqn . ( 2 \text{-} 4 )$$

where Lₚ is the hydraulic conductivity, S/V is the surface area of the capillaries per volume of the brain, a is the reflection coefficient, P and IF are pressures, vₛ is the velocity at the brain surface, and Pᵢ is represented by Eqn. (2-5) below:

$${\overline{P}}_{i} = \frac{{P}_{i} - {P}_{SAS}}{{P}_{ventricle} - {P}_{SAS}} \qquad Eqn . ( 2 \text{-} 5 )$$

After non-dimensionalizing the momentum balance equation and ignoring higher order terms, the momentum equation reduces to Eqn. (2-6) (Arifin et al, 2009, Pharma. Research, 26:2289):

$${\overline{v}}_{x} = \frac{d {\overline{P}}_{i}}{d \overline{x}} \qquad Eqn . ( 2 \text{-} 6 )$$

Combining the dimensionless continuity and momentum equations reduces to Eqn. (2-7):

$${\overline{P}}_{i} = A {e}^{- \sqrt{α} \overline{x}} + B {e}^{\sqrt{α} \overline{x}} + \frac{β}{α} \qquad Eqn . ( 2 \text{-} 7 )$$

where α and β may be represented by Eqns. (2-8) and (2-9):

$$\begin{matrix}α = \frac{L}{{v}_{s}} {L}_{P} ( S \text{/} V ) ( {P}_{ventricle} - {P}_{SAS} ) & Eqn . ( 2 \text{-} 8 ) \\ β = \frac{L}{{v}_{s}} {L}_{P} ( S \text{/} V ) [ {P}_{vascular} - {P}_{SAS} - σ ( {π}_{vascular} - {π}_{i} ) ] & Eqn . ( 2 \text{-} 9 )\end{matrix}$$

This shows that the flow is pressure driven without substantial viscous losses other than due to the porosity alone. The velocity may be calculated from the now-known pressure profile:

$${\overline{v}}_{x} = \frac{d }{d x} ( A {e}^{- \sqrt{α} \overline{x}} + B {e}^{\sqrt{α} \overline{x}} + \frac{β}{α} ) = \sqrt{α} ( A {e}^{- \sqrt{α} \overline{x}} - B {e}^{\sqrt{α} \overline{x}} ) , \qquad Eqn . ( 2 \text{-} 10 )$$

where A and B may be represented by Eqns. (2-11) and (2-12):

$$\begin{matrix}A = 1 - B - \frac{β}{α} & Eqn . ( 2 \text{-} 11 ) \\ B = \frac{\frac{β}{α} ( {e}^{- \sqrt{α}} - 1 ) - {e}^{- \sqrt{α}}}{{e}^{\sqrt{α} - {e}^{- \sqrt{α}}}} & Eqn . ( 2 \text{-} 12 )\end{matrix}$$

Using Eqns. (2-7) and (2-10), the pressure and velocity have the profiles across the brain shown in FIG. 13. FIG. 13 illustrates pressure and fluid velocity changes from the surface of the ventricles (x=0) to the surface of the brain (x=1).

For transport of an Aβ peptide, the mass balance is (neglecting diffusion due to the P):

$$\frac{\partial A β}{\partial t} + {v}_{x} \frac{\partial A β}{\partial x} = {D}_{BA} \frac{{\partial }^{2} A β}{\partial {x}^{2}} + {kC}_{99} - \dot{V} A β , \qquad Eqn . ( 2 \text{-} 13 )$$

where k_{C99} is the rate of creation of C99 and \dot{V}_{Aβ} is the rate of irreversible loss of Aβ.

After partially non-dimensionalizing the steady state equation, the effects of diffusion and time dependent terms may be neglected without introducing substantial error, resulting in the steady state equation below:

$$\frac{\partial A β}{\partial \overline{x}} = \frac{L}{{v}_{s} {\overline{v}}_{x}} ( {kC}_{99} - \dot{V} A β ) \qquad Eqn . ( 2 \text{-} 14 )$$

The expression for velocity as a function of x calculated in Eqn. (XI) was inserted into Eqn. (2-15) and integrated with a boundary condition of Aβ(0)=0 which yielded the Aβ steady state equation below:

$$A {β}_{ss} = \frac{{kC}_{99}}{V} ( 1 - {e}^{( - \frac{L \dot{V}}{{v}_{s} α \sqrt{AB}} [ {tanh}^{- 1} ( \frac{B {e}^{\sqrt{α} \overline{x}}}{\sqrt{AB}} ) - {tanh}^{- 1} ( \frac{B}{\sqrt{AB}} ) ] )} ) \qquad Eqn . ( 2 \text{-} 15 )$$

The ‘brain’ was divided into 100 equally spaced nodes and the unsteady system of differential equations was solved numerically for iAPP, mAPP, C99 immobilized in the brain, Aβ38, Aβ40, and Aβ42 in the interstitial fluid and CSF, and Aβ42 in the exchange compartment (705 equations).

The results of this experiment demonstrated that the one-dimensional flow of Aβ in the brain may be modeled.

### Example 5

### The One-Dimensional Flow Model was Assessed

To assess the model of one-dimensional flow of Aβ in the brain, the following experiment was performed. The model of Examples 1 and 4 were used to model patients that were normal controls (NC) PSEN1 or PSEN2 mutation carriers that were both PIB positive (MC+) and negative (MC−).

The rate of production of labeled iAPP was the product of the rate constant k_{iAPP} with the fractional labeling of leucine amino acid. This value was set to 25 h⁻¹ for all patients. For the production rate constant of mAPP and C99, different values were investigated while fitting the data to one of the patients with plaques detectable by PET. In Example 2, six out of seven of the patients with plaques required exchange of Aβ42 to optimally fit the data, and many required a large amount of exchange. This is due to the characteristic shape of the curves (FIG. 13). The exchange process only had a substantial effect on the labeling curve if the rates of clearance of the Aβ peptides (e.g. \dot{V}₃₈, \dot{V}₄₀, or \dot{V}₄₂) were lower than about 0.25 h⁻¹. Turnover of Aβ peptides could only be that slow if the turnover of mAPP and C99 were relatively high. Because the data likely had little information about k_{mAPP} and k_{C99} independently, these two parameters were set equal. Systematically varying the rate constant for k_{mAPP} and k_{C99} while fitting the plaque-bearing patient led to optimal values of 1.2 h⁻¹ for L=3 cm and 1.6 h⁻¹ for L=7 cm. Ranges of other parameter values were fixed based on the findings of the previous model, and the ranges were expanded when the optimized parameter reached a prescribed limit. Tables 3 and 4 show values for parameters used in the one-dimensional flow model.

TABLE 3

| | Lower Limit | Upper Limit |
|---|---|---|
| k_{iAPP} | 25 | |
| k_{mAPP}, k_{C99} | 1.2 (L = 3 cm); 1.6 (L = 7 cm) | |
| \dot{V}_{C99} | 0.001 | 1.3 |
| k_{Aβ38}, k_{Aβ40} and k_{Aβ42} | Calculated from steady state relationship | |
| \dot{V}₃₈, \dot{V}₄₀, or \dot{V}₄₂ | 0.01 | 0.3 |
| kₑₓ₄₂ | 1 × 10⁻⁸ | 1 |
| k_{delay} | 0.05 | 2 |
| SF₃₈, SF₄₀, SF₄₂ | 0.7 | 1.3 |

TABLE 4

| | \dot{V}_{C99} | k_{Aβ38} | k_{Aβ40} | k_{Aβ42} | k_{Aβ42}/k_{Aβ40} |
|---|---|---|---|---|---|
| NC | 0.40 ± 0.35 | 0.0052 ± 0.0043 | 0.020 ± 0.016 | 0.0028 ± 0.0022 | 0.142 ± 0.00999 |
| MC− | 0.56 ± 0.21 | 0.0099 ± 0.0039 | 0.033 ± 0.0086 | 0.062 ± 0.0022* | 0.185 ± 0.0167** |
| MC+ | 0.31 ± 0.13 | 0.0024 ± 0.00065 | 0.0098 ± 0.0028 | 0.0017 ± 0.00055 | 0.168 ± 0.0198** |

| | \dot{V}₃₈ | \dot{V}₄₀ | \dot{V}₄₂ | \dot{V}₄₂/\dot{V}₄₀ | kₑₓ₄₂ | k_{delay} |
|---|---|---|---|---|---|---|
| NC | 0.18 ± 0.056 | 0.19 ± 0.058 | 0.18 ± 0.053 | 0.957 ± 0.0894 | 0.0084 ± 0.015 | 0.76 ± 0.45 |
| MC− | 0.17 ± 0.050 | 0.17 ± 0.053 | 0.22 ± 0.077 | 1.28 ± 0.343** | 0.035 ± 0.024* | 0.32 ± 0.058 |
| MC+ | 0.12 ± 0.037* | 0.11 ± 0.035** | 0.19 ± 0.050 | 1.71 ± 0.293** | 0.14 ± 0.10** | 0.84 ± 0.39 |

| | SF38 | SF40 | SF42 |
|---|---|---|---|
| NC | 0.85 ± 0.069 | 0.85 ± 0.051 | 0.91 ± 0.092 |
| MC− | 0.83 ± 0.043 | 0.85 ± 0.061 | 0.82 ± 0.016 |
| MC+ | 0.91 ± 0.085 | 0.92 ± 0.14 | 0.88 ± 0.13 |

The ratio of the rate constant for the production of Aβ42 with respect to the rate constant for the production of Aβ40 was highly significant when comparing both the MC− and MC+ groups to the normal controls (NC). However, the MC− and MC+ groups were not different from each other.

The ratio of the rate constant for the permanent loss of Aβ42 (\dot{V}₄₂) with respect to the rate constant for the permanent loss of Aβ40 (\dot{V}₄₀) was also highly significant when comparing both the MC− and MC+ groups to the normal controls. Although it was expected that only the MC+ group should show increased loss of Aβ42 relative to Aβ40, it is possible that some patients in the MC− group were beginning to deposit plaques, but these were not yet detectable by PIB. This is supported by the significant increase in the exchange rate constant in the MC− group (p=0.19), although the mean was nearly four-fold smaller than in the MC+ group. The rate constant for permanent loss was 33% higher in the MC+ compared the MC− group, and this difference trended towards significance (p=0.057). Interestingly, the increased \dot{V}₄₂/\dot{V}₄₀ ratio in the MC+ group seemed to be due to a significant decrease in \dot{V}₄₀ rather than an increase in \dot{V}₄₂. This result is in agreement with the findings of the purely compartmental model of the data in Examples 1 and 2. However, in this model, the rate constant for the clearance of Aβ38 (\dot{V}₃₈) is also significantly lower in the MC+ group. This may represent a general decrease in clearance of from the brain in the presence of plaques, perhaps due to changes in the physiology of the brain.

Compared to the model in Examples 1 and 2, the AIC was lower in the Example 2 model in 13/23 patients, and was lower in the current model in 10/23 patients. However, the AIC were quite similar, with the sum of AIC over all the patients of −25,818.2 for the previous model and −25,729.7 for the current model.

In the current model, only exchange of Aβ42 is allowed, and this parameter is allowed to vary in all patients. In the Example 2 model, patients were allowed to exchange Aβ peptides only if it improved the AIC. The current model treats the exchange rate constant as a continuous variable, this facilitates comparison of this parameter with other measures of Alzheimer's disease. In particular, the correlation between the exchange rate constant and the PIB score is presented. The correlation coefficient of r=0.851 indicates high correlation between the two measures. In contrast, the correlation coefficient between the predicted brain pool size of Aβ42 and the exchange rate constant was r=−0.441. This indicates some relationship between these variables.

The results of this experiment demonstrate that the model may represent one-dimensional flow of Aβ in the brain.

### Example 6

### Method of Calibrating a Differential Aβ Isoform Kinetics Model

In one embodiment, the computing device 102 or client 108 executes the MCA 104 in response to a modeling request from the user. The user identifies one or more patients for whom Aβ modeling will be calibrated using the input device 120 and one or more GUI's generated by the GUI module 300.

A GUI module 300 receives data from the various other modules 302-310, the input device 120, and/or the data source 106 and generates one or more displays on the display device 116. The displays generated by the GUI module may include input forms, charts, graphs, displays, tables, and other data for viewing by the user of the MCS 100.

In response, the patient data module 302 generates a request to retrieve patient data. In one embodiment, the request is transmitted to the data source 106 to retrieve patient data. The patient data may include biographical data as well as medical data for the identified patient. The patient data may also identify a diseased state of a patient. The patient data may further include baseline data values related to one or more component levels within the patient's blood, CSF, or other baseline data of interest. Alternately, if the MCA 104 is being executed contemporaneously with a new patient, the request for patient data may be transmitted to the GUI module 302, where one or more GUI's and data entry fields are generated for display on the display device 116 for the user to input baseline values, which are received at the patient data module 302.

Once baseline values for the patient have been established, the MCA 104 determines a plasma leucine enrichment value for the patient. The plasma leucine enrichment value is calculated by referencing known data enrichment values as a function of time, as shown in FIG. 5 and comparing the known data to the patient data obtained at the patient data module 302.

As previously described, a time-dependent delay compartment of the model is used to represent the uptake of the labeled plasma leucine by APP and the subsequent formation of the Aβ isoforms by cleaving C99 peptides. As such, the MCA 104 includes an Aβ isoforms module 304 that determines the level of each Aβ isoform after cleavage, which incorporates the labeled leucine. The Aβ isoforms module 304 determines the amounts or values for each labeled isoform as well as each isoform's respective enrichment levels by first multiplying the determined plasma labeled leucine level by an uncalibrated APP constant (k_{APP}), as identified in Table 1, to obtain an uncalibrated level of enriched C99 peptides. The exemplary uncalibrated APP constant is retrieved from a table of mean data values stored in the data source 106. Similarly, the Aβ isoforms module 304 determines an exemplary level for each Aβ isoform entering the CSF by multiplying the calibrated level of enriched C99 peptides by a mean transfer rate values for each respective isoform cleaved from C99 peptides. This determination also accounts for a certain level of the C99 peptides that are lost and not converted to the Aβ isoforms by using an exemplary irreversible loss C99 constant (V_{c99}).

In one embodiment, the Aβ isoforms module 304 may also be used to calibrate and quantify the state-state kinetics of isoforms. For example, the model may be used to model the kinetics of the Aβ38, Aβ40, and Aβ42 isoforms.

In one aspect, the Aβ isoforms module 304 may be used to determine if an exchange compartment is necessary to model the kinetics of the “soluble” peptides. The module 304 optimizes the model by creating the exchange compartment in response to a determination that the added exchange process improves the Akaike Information Criteria (AIC) for a curve fit. For example, data from exemplary modeling performed using SAAM II software may be stored in the data source 106. In particular, the user or the MCA 104 may automatically incorporate one or more exchange compartments into the exemplary model to calibrate and improve the correspondence between the sigmoid shapes of the enriched Aβ-isoforms within the CSF with respect to time as compared to data in the data source 106.

When exchange compartments are used, the Aβ isoforms module 304 multiplies the previously calculated isoform levels by an exemplary exchange rate (Kₑₓ) and an exemplary return rate (Kᵣₑₜ). The exchange compartments and rate factors Kₑₓ and Kᵣₑₜ are used to represent the possible recycling of Aβ isoforms to and/or from amyloid plaques, the exchange of labeled Aβ for unlabeled Aβ, the recycle of higher order Aβ structures, and other as of yet unknown losses and gains to the levels of the respective isoforms.

In addition, the Aβ isoforms module 304 may multiply the calculated isoform levels by one or more scaling factors to account for small amounts of isotopic dilution between plasma leucine and the biosynthetic precursor pool (generally <5%) or to correct for minor calibration errors (generally <10%) in the measurement of isotope enrichments of plasma leucine and/or Aβ peptides.

The CSF isoform module 306 receives data related to the levels of each respective isoform within the CSF. In one aspect, the CSF isoform module 306 receives data regarding the measured or calculated isoform levels after cleavage from the C99 peptide, and/or levels calculated from one or more optional exchange compartments. In addition, the CSF isoform module 306 may be used to predict the levels of each isoform within the CSF as a function of time by multiplying the received data by an exemplary delay factor (K_{delay}). As shown in the kinetic model 20, K_{delay} may be used to represent the perfusion of labeled peptides through various brain tissue and heterogeneous CSF fluid transport processes.

The results module 308 processes data transmitted from the data source 106 and/or one or more other modules 300-306, and 310 to generate a display of results generated by the kinetic model 20. In one example, the results module 308 may generate a chart or other graphical representation of data values, while the GUI module 302 generates a display of the representation.

The calibration module 310 allows the user to modify one or more of the rate constants or other constants used in the kinetic model 20. In one aspect, the calibration module 310 in conjunction with the GUI module 300 and/or the results module 308 generates one or more GUIs that a user may interact with to modify the parameters of the model, the data values generated by the model, and/or the graphical representation of the data values. By way of example and not limitation, the calibration module 310 may receive data input into a GUI using the input device 120 to modify a constant value of the kinetic model 20. This input data may be used to modify one or more graphical representations generated by the results module 308. As such, the user may vary the data values generated by the kinetic model 20, which contemporaneously varies the graphical representation of the data in order to calibrate the model data values to the measured data value.

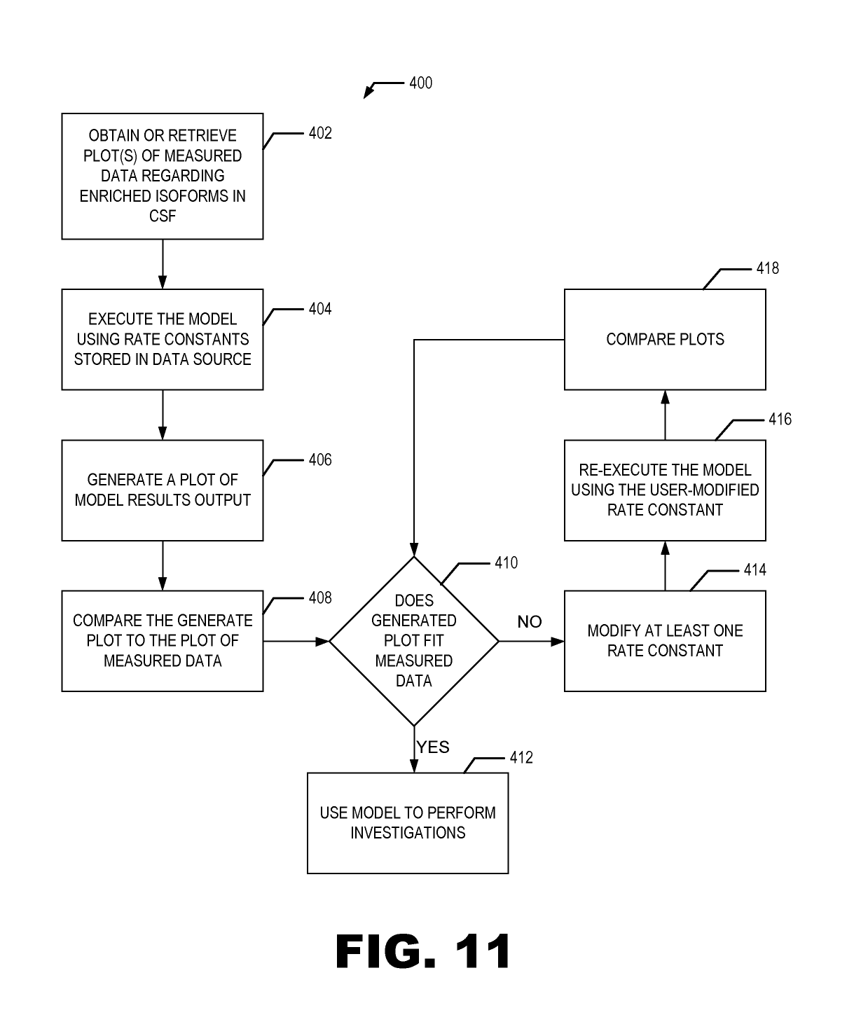

FIG. 11 is a flowchart illustrating a method 400 of calibrating the kinetic models 10, 20, or 50, shown in FIGS. 3, 4, and 21 according to one embodiment. At 402, leucine enrichment and labeled isoform level data values, as previously described, are collected and plotted for one or more patients. Alternately, previously collected or plotted data may be retrieved from a data source. At 404, the compartment model is executed using known or measured leucine enrichment data and rate constants stored in the data source. At 406, plots of the model results are generated and, at 408, the generated plots are compared to the plots previously retrieved or created at 402.

A determination regarding the fit or closeness of fit between the plots of measured data and the plots generated by the model is made at 410. If the model-generated plots are determined to sufficiently fit the plots of measured data, the model may be deemed calibrated and used as a tool in other investigations at 412. Conversely, if the model-generated plot does not fit the plots of measured data, then one or more of the rate constant values may be modified at 414 and the model may be re-executed at 416. Similar to the comparison made at 408, the plot generated by the model using the modified rate constant(s) is compared to the plot of the measured data from 402 at 418. Another determination is made at 410 to determine if the “modified rate constant” plot sufficiently fits the plot of measured data. The process at 410-418 may be repeated as necessary, until the user is satisfied with the calibration of the model. In various embodiments, the same rate constant, different rate constants, or combinations thereof may be modified at 414.

The description above includes example systems, methods, techniques, instruction sequences, and/or computer program products that embody techniques of the present disclosure. However, it is understood that the described disclosure may be practiced without these specific details. In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes.

### Example 7

### Outcomes Apparent in the Raw Data are Independent of the Type of Mathematical Model that Might be Used to Describe the Data

The kinetic tracer curves for Aβ42 are known to differ compared to other index peptides (for example, Aβ38 and Aβ40) for certain patient populations. The data reflect the involvement of plaques, as evidenced by PIB scores. A compartmental model was developed as one way of extracting kinetic parameters from the experimentally measured data. Numerous models may be used to describe the data, and it is predicted that all such models will reveal differences in Aβ42 kinetics if they provide satisfactory fits to the data. The following summarizes outcomes apparent in the raw data itself that are independent of the type of compartmental or non-compartmental model that might be used to describe the data, and demonstrates that the SILK tracer kinetic protocol reveals differences in Aβ42 kinetics that will be diagnostic of plaques.

### Example 8

### SILK Tracer Kinetic Protocol Reveals Differences in Aβ42 Kinetics that May be Diagnostic of Plaques

The kinetic tracer curve for Aβ42 and the other index peptides (e.g. Aβ38, Aβ40) was different during several different phases of the curve in the presence of plaques. FIGS. 6A-6F show the major differences in the Aβ42 kinetic time course compared to Aβ38 and Aβ40. The different phases, or aspects, of the kinetic tracer curves to focus on are: (i) Initial rise, which is the front-end slope of the curve, and also described as the “fractional synthesis rate” (FSR) as calculated in the Science 2010 paper); (ii) Peak time; (iii) Peak enrichment; (iv) Initial downturn monoexponential slope; and (v) Terminal monoexponential slope, which is the back-end slope of the curve between 24-36 hours, and is also described as the “fractional catabolic rate” FCR as calculated in the Science 2010 paper). The particular Aβ42 features in the presence of plaques to focus on are: (i) Initial rise—Aβ42 might be faster; (ii) Peak time—Aβ42 peaks earlier; (iii) Peak enrichment—Aβ42 peaks lower; (iv) Initial downturn monoexponential slope—initial Aβ42 slope may be faster; and (v) Terminal monoexponential slope—terminal Aβ42 slope may be slower. Each outcome is discussed in further detail below.

(i) Fractional Synthesis Rate (Uses 6-12 h TTR Slope and Plasma Leucine TTR Enrichment)

None of the Aβ peptide (ABxx) FSRs discriminate PIB status or correlate with PIB score. The Aβ42/Aβxx ratios are lower in PIB+ group (significant when Aβ38 or Total AB is used for normalization, but not when AB40 is used). The Aβ42/38 FSR ratio is significantly negatively correlated with PIB score, but a P value of 0.028 is not that impressive in comparison to other outcomes (see below). FIGS. 6A-6F show that the Aβ42 enrichment is higher than Aβ38 or Aβ40 during the early rise. However, Aβ42 enrichment also rises out of the background a little earlier, and thus the early Aβ42 enrichment has an upward offset without a faster early slope. The 6-12 h time points were used for this FSR analysis. A different range of time points might show a significant difference. However, a practical issue to keep in mind is to balance having enough data points to adequately filter out noise in the data, against having too many points such that a linear slope is being fit to a sigmoidal rise peak. The front end of Aβ42 may not be significantly diagnostic of plaque involvement.

TABLE 5
Initial ratio of rise (6-12 h slope FSR)

| | | | | FSR | | | |
|---|---|---|---|---|---|---|---|
| | FSR | FSR | FSR | Total | | | |
| | 38 | 40 | 42 | AB | 42/38 | 42/40 | 42/total |
| | pools/h | pools/h | pools/h | pools/h | ratio | ratio | ratio |
| PIB− group: | | | | | | | |
| Mean | 0.0453 | 0.0466 | 0.0481 | 0.0449 | 1.071 | 1.033 | 1.067 |
| StDev | 0.0114 | 0.0110 | 0.0120 | 0.0094 | 0.132 | 0.101 | 0.112 |
| PIB+ group: | | | | | | | |
| Mean | 0.0457 | 0.0448 | 0.0411 | 0.0437 | 0.910 | 0.939 | 0.947 |
| StDev | 0.0134 | 0.0161 | 0.0123 | 0.0119 | 0.152 | 0.152 | 0.154 |
| P, 2-tailed t | | | | | | | |
| tests: | | | | | | | |
| PIB− vs. PIB+ | .94 | .76 | .22 | .79 | 0.018 | .09 | 0.048 |
| Correlations vs. | | | | | | | |
| PIB score: | | | | | | | |
| Correlation | −0.075 | −0.146 | −0.341 | −0.180 | −0.459 | −0.355 | −0.358 |
| coefficient: | | | | | | | |
| P value: | .73 | .51 | .11 | .41 | 0.028 | .10 | .09 |

(ii) Time to Peak

None of the individual Aβ peptide peak times discriminate between PIB groups or are significantly correlated against PIB score. However, Aβ42 peaks significantly earlier than either Aβ38, Aβ40, or total AB in the PIB+, and the ratios of the peak times is very highly significantly correlated with the PIB score. Thus, the degree to which the Aβ42 peak is shifted earlier correlates with plaque involvement.

TABLE 6
Time to peak

| | | | | Peak | | | |
|---|---|---|---|---|---|---|---|
| | Peak | Peak | Peak | Time | | | |
| | Time | Time | Time | Total | | | |
| | 38 | 40 | 42 | AB | 42/38 | 42/40 | 42/total |
| | h | h | h | H | ratio | ratio | ratio |
| PIB− group: | | | | | | | |
| Mean | 17.7 | 17.6 | 17.6 | 17.5 | 0.994 | 1.000 | 1.007 |
| StDev | 1.4 | 1.4 | 1.4 | 1.4 | 0.033 | 0.032 | 0.032 |
| PIB+ group: | | | | | | | |
| Mean | 17.9 | 18.0 | 16.3 | 17.9 | 0.907 | 0.903 | 0.908 |
| StDev | 1.6 | 1.6 | 1.5 | 1.8 | 0.042 | 0.043 | 0.050 |
| P, 2-tailed | | | | | | | |
| t tests: | | | | | | | |
| PIB− vs. PIB+ | .76 | .56 | .05 | .53 | 2.45E−05 | 4.85E−06 | 1.13E−05 |
| Correlations vs. | | | | | | | |
| PIB score: | | | | | | | |
| Correlation | 0.131 | 0.178 | −0.359 | 0.203 | −0.776 | −0.792 | −0.794 |
| coefficient: | | | | | | | |
| P value: | .55 | .42 | .09 | .35 | 1.38E−05 | 6.67E−06 | 6.18E−06 |

(iii) Peak Enrichment

In these data, enrichment is measured as tracer-to-tracee ratio, but other units of enrichment could be used instead. By itself, the peak enrichment of Aβ42 discriminates between PIB+/− groups and is significantly negatively correlated with PIB score (higher PIB score=lower peak enrichment); but the P value of 0.016 on this is not strongly significant. The lower Aβ42 enrichment is much more strongly associated with plaques when it is normalized to the other index proteins, either Aβ38, 40, or total Aβ. This normalization is crucial as it controls for variability in the plasma leucine enrichment plateau between subjects that is observed with the SILK protocol.

TABLE 7
Peak Enrichment

| | | | | Peak | | | |
|---|---|---|---|---|---|---|---|
| | | | | Max | | | |
| | Peak | Peak | Peak | Total | | | |
| | Max 38 | Max 40 | Max 42 | AB | 42/38 | 42/40 | 42/total |
| | TTR | TTR | TTR | TTR | ratio | ratio | ratio |
| PIB− group: | | | | | | | |
| Mean | 0.0879 | 0.0896 | 0.0912 | 0.0874 | 1.040 | 1.018 | 1.042 |
| StDev | 0.0139 | 0.0137 | 0.0149 | 0.0119 | 0.085 | 0.063 | 0.077 |
| PIB+ group: | | | | | | | |
| Mean | 0.0842 | 0.0818 | 0.0724 | 0.0805 | 0.867 | 0.891 | 0.903 |
| StDev | 0.0152 | 0.0149 | 0.0123 | 0.0133 | 0.102 | 0.090 | 0.088 |
| P, 2-tailed t | | | | | | | |
| tests: | | | | | | | |
| PIB− vs. PIB+ | .56 | .23 | 0.0081 | .23 | 3.97E−04 | 7.98E−04 | 9.61 E−04 |
| Correlations | | | | | | | |
| vs. PIB | | | | | | | |
| score: | | | | | | | |
| Correlation | −0.051 | −0.168 | −0.495 | −0.193 | −0.692 | −0.714 | −0.649 |
| coefficient: | | | | | | | |
| P value: | .82 | .44 | 0.0164 | .38 | 2.51 E−04 | 1.28E−04 | 8.11 E−04 |

(iv) Initial Monoexponential Slope FCR

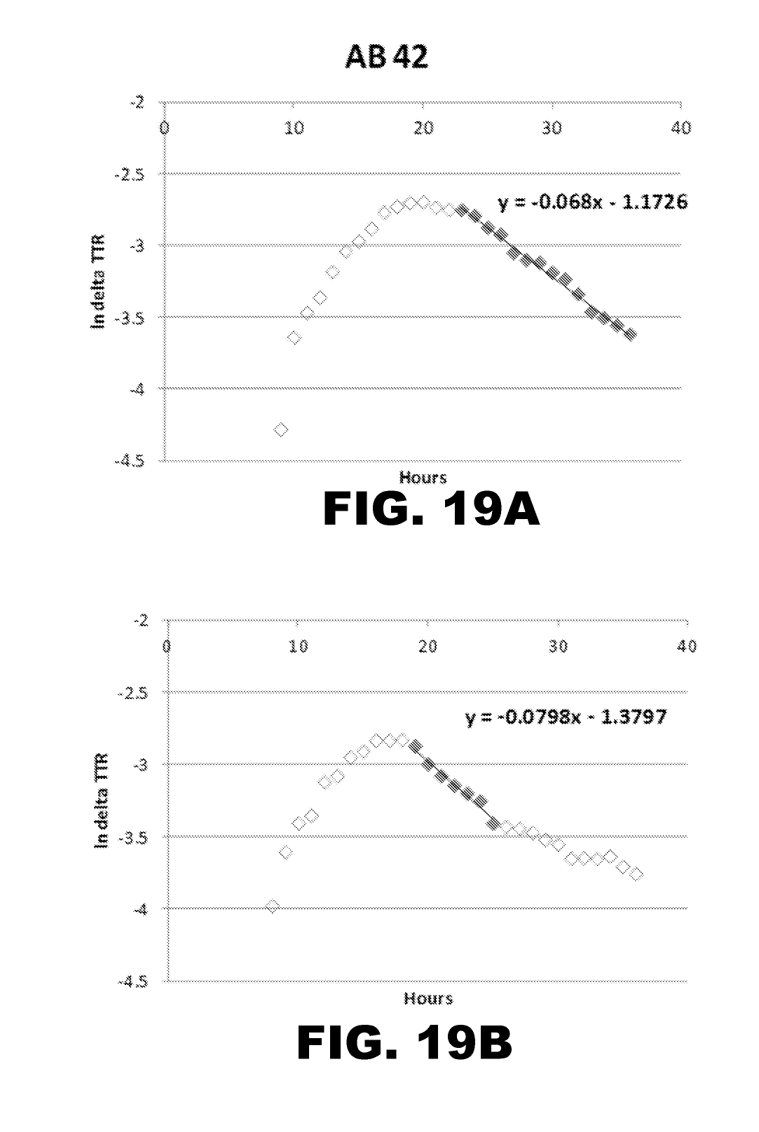

A monoexponential slope is fit to the descending enrichment on the back end of the time course. In most studies, the entire back end of the peak is monoexponential to the end of the time course (36 h) as shown in FIG. 19A. However, in many cases there is evidence of a 2nd, slower exponential tail to the peak as shown FIG. 19B; in these cases, an initial rapid slope that visually excludes the slower tail is selected. The plots show the natural log of enrichment vs. time; the monoexponential slope FCR is the negative of the slope.

None of the individual peptide monoexponential slopes significantly discriminate between PIB groups, although there is a trend that Aβ40 and total Aβ have slower slopes in the PIB+ group. Greater discriminatory power is achieved by looking at the correlation against PIB score, where the monoexponential slopes for Aβ38, Aβ40, and total Aβ are all significantly negatively correlated against PIB score (slower slope in relation to the degree of plaque quantity). In the formal compartmental model, this came out as a decreased fractional turnover rate (FTR) of soluble Aβ38 & Aβ40 in the brain in the presence of plaques.

However, the Aβ42 monoexponential slope does not significantly discriminate between PIB groups nor does it correlate with PIB score. The FTR of soluble Aβ38 and Aβ40 was slowed down in the presence of plaques. This turnover is largely due to fluid perfusion through the brain, and we propose that the fluid perfusion rate is slowed down in the presence of plaques. In the compartmental model, it is assumed that the FTR of Aβ42 that is due to the fluid perfusion process would be the same as it is for Aβ38 and Aβ42. Since the initial monoexponential slope of Aβ42 is not significantly slower in the presence of plaques, but it should be if fluid perfusion was the sole process for Aβ42 turnover, we therefore concluded that some other process of irreversible loss was causing the total FTR of Aβ42 (fluid perfusion loss+extraneous loss) to be increased selectively in the PIB+ group. We take this as kinetic evidence for removal of soluble Aβ42 from the brain fluid and deposition into plaques, which accounts for the observation that the initial monoexponential is not slower in the presence of plaques (even though the slopes of Aβ38 & Aβ40 are slower), and also provides a mechanism that reduces the concentration of AB42 relative to Aβ38 or Aβ40 that is recovered in CSF.

The Aβ42 initial monoexponential slope also fails to discriminate between PIB groups or correlate with PIB score when it is normalized using either Aβ38, Aβ40 or total Aβ as a reference. Thus, in conclusion, the initial monoexponential slope FCR of Aβ42 is not diagnostic of plaques.

TABLE 8
Initial monoexponential slope FCR

| | | | | Total | | | 42/ |
|---|---|---|---|---|---|---|---|
| | AB 38 | AB 40 | AB 42 | AB | 42/38 | 42/40 | total |
| | /h | /h | /h | /h | ratio | ratio | ratio |
| PIB− group: | | | | | | | |
| Mean | 0.0937 | 0.0963 | 0.0986 | 0.0948 | 1.051 | 1.024 | 1.040 |
| StDev | 0.0179 | 0.0182 | 0.0221 | 0.0181 | 0.098 | 0.107 | 0.102 |
| PIB+ group: | | | | | | | |
| Mean | 0.0815 | 0.0794 | 0.0896 | 0.0793 | 1.139 | 1.165 | 1.154 |
| StDev | 0.0204 | 0.0183 | 0.0175 | 0.0181 | 0.260 | 0.272 | 0.211 |
| P, 2-tailed t | | | | | | | |
| tests: | | | | | | | |
| PIB− vs. | .16 | .05 | .35 | .07 | .24 | .08 | .09 |
| PIB+ | | | | | | | |
| Correlations | | | | | | | |
| vs. PIB | | | | | | | |
| score: | | | | | | | |
| Correlation | −0.418 | −0.494 | −0.297 | −0.480 | 0.288 | 0.363 | 0.371 |
| coefficient: | | | | | | | |
| P value: | 0.0473 | 0.0167 | .17 | 0.0204 | .18 | .09 | .08 |

(v) Terminal Monoexponential Slope FCR

A monoexponential slope was fit to t=24-36 h of the time course as reported in the Science 2010 paper; this is done without regard for whether the peak exhibits monoexponential or biexponential behavior (see natural log plots in FIGS. 19A-19B for illustration).

By itself, the terminal slope of Aβ42 very weakly discriminates between PIB groups (P=0.0355), with PIB+ having a slower terminal tail. In the model, this is accounted for by the “exchange compartment” whereby newly synthesized (i.e., labeled) Aβ42 enters into an exchange process that returns labeled Aβ42 to the soluble pool later, which is a feature of tracer recycling that causes a flattening of the terminal tail. The terminal slopes of Aβ38 or Aβ40 do not discriminate between PIB groups. The terminal slopes of all 3 peptides, however, are significantly negatively correlated with PIB score, which results from the feature described above whereby the turnover of soluble Aβ peptides may be mostly driven by fluid transport through the brain tissue, and this transport process is retarded in the presence of plaques. The small degree of discrimination between groups for Aβ42 is lost when that slope is normalized to either the Aβ38 or Aβ40 slope. In conclusion, the terminal monoexponential slope of Aβ42 is not particularly diagnostic for plaques. There is a weak power to discriminate, but the enrichment measurements are somewhat noisy (especially as enrichments get lower toward the end of the protocol), and the slope is not all that useful.

TABLE 9
Terminal slope FCR (24-36 h slope)

| | Terminal | Terminal | Terminal | | |
|---|---|---|---|---|---|
| | slope | slope | slope | | |
| | FCR38 | FCR40 | FCR42 | 42/38 | 42/40 |
| | pools/h | pools/h | pools/h | ratio | ratio |
| PIB− group: | | | | | |
| Mean | 0.0844 | 0.0851 | 0.0848 | 1.005 | 0.994 |
| StDev | 0.0115 | 0.0112 | 0.0150 | 0.104 | 0.085 |
| PIB+ group: | | | | | |
| Mean | 0.0765 | 0.0761 | 0.0689 | 0.902 | 0.908 |
| StDev | 0.0150 | 0.0166 | 0.0171 | 0.199 | 0.183 |
| P, 2-tailed | | | | | |
| t tests: | | | | | |
| PIB− vs. PIB+ | .18 | .14 | 0.0355 | .12 | .13 |
| Correlations | | | | | |
| vs. PIB score: | | | | | |
| Correlation | −0.422 | −0.472 | −0.462 | −0.194 | −0.139 |
| coefficient: | | | | | |
| P value: | 0.0451 | 0.0229 | 0.0265 | .37 | .53 |

(vi) Overall Conclusions

The peak time and peak enrichment of Aβ42 is very highly significantly associated with plaques: Aβ42 peaks earlier and lower when plaques are present. The slope on the front end and the initial and terminal monoexponential slopes on the back end are not particularly sensitive to the presence of plaques.

The presence of plaques clearly alters biologic processes that distinguish the Aβ42 turnover curve from Aβ38, Aβ40, or total Aβ. The earlier and lower peak of Aβ42 in the presence of plaques (peak time and peak enrichment, respectively) causes a separation of enrichments on the back end of the curve (see time course plots). In addition to these two measurements, recent results show that a comparison of isotopic enrichments around the midpoint on the back end of the curve (˜24 h) is also able to discriminate the PIB groups highly significantly. A fourth measurement that may be associated with plaques is the degree to which Aβ42 enrichment on the descending peak is different from Aβ38, Aβ40, or Total Aβ enrichment.

### Example 9

### Additional In Vivo Data Using the SILK Tracer Kinetic Protocol

It was hypothesized that simple measures that summarize some aspect of the SILK tracer curve of amyloid beta (Aβ) may provide diagnostic or prognostic information about patients with AD, at risk of AD, or suspected of having AD. To test the above hypothesis, discrimination between the three groups of patients was attempted based on the ratio of the percent of Aβ42 labeling to the percent of Aβ40 percent calculated during the downturn of the Aβ SILK tracer curve. In vivo SILK studies were performed in patients with PSEN1 or PSEN2 mutations that were PIB positive by PET (MC+), patients with PSEN1 or PSEN2 mutations that were PIB negative by PET (MC−), and non-carrier mutation carrier sibling controls (NC) as described elsewhere in U.S. Pat. No. 7,892,845, which is hereby incorporated herein in its entirety. Briefly, subjects were administered isotope-labeled leucine (¹³C₆-leucine) for 9 hours via intravenous infusion. CSF samples (6 mL/sample) were collected 23 hours and 24 hours after the start of the infusion of labeled amino acid. Quantitative measurements of labeled and unlabeled Aβ42 and Aβ40 were obtained by tandem mass spectrometry, and the ratio of labeled:unlabeled Aβ42 and labeled:unlabeled Aβ40 was calculated for each timepoint. These ratios represent the percent labeled of each Aβ isoform at 23 hours and 24 hours post infusion.

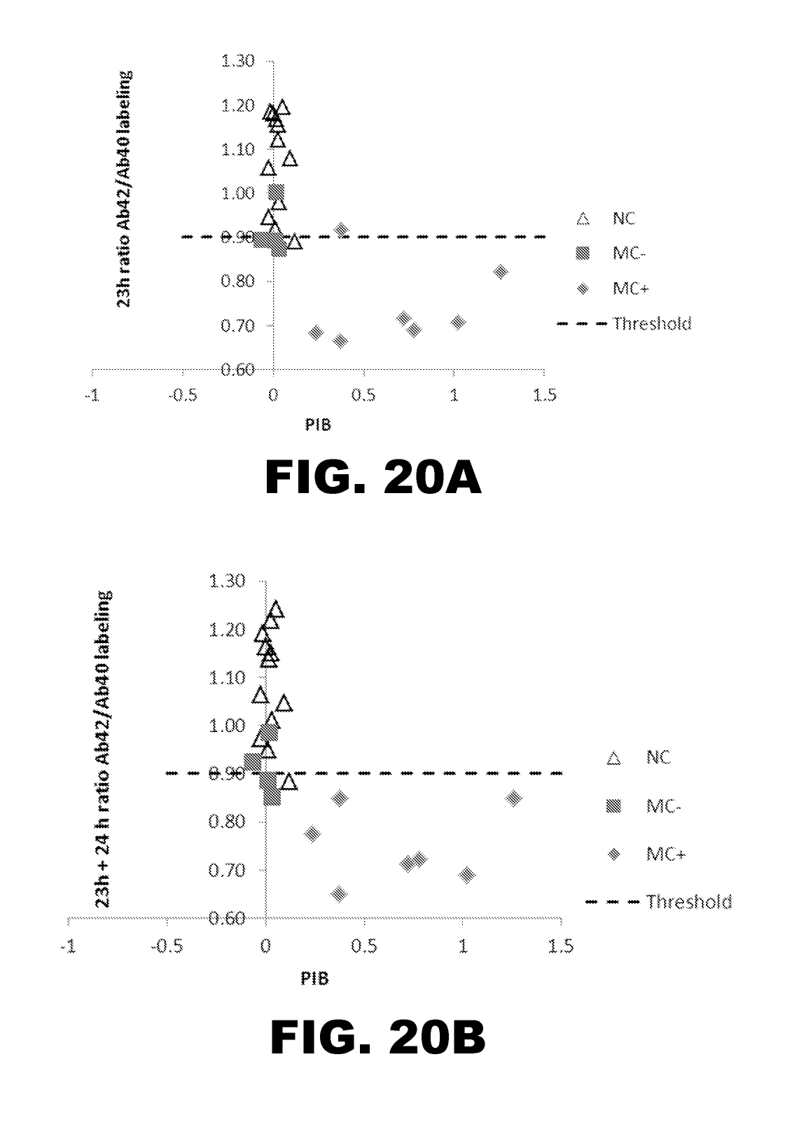

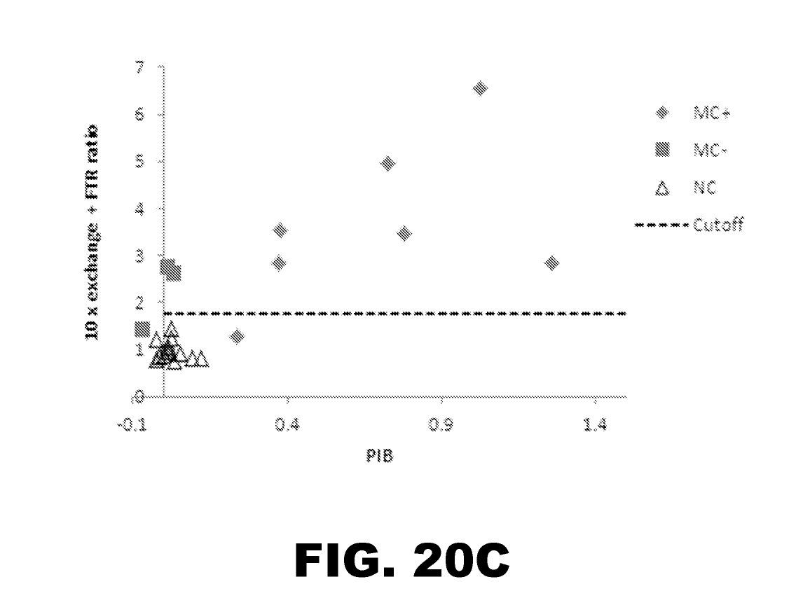

A diagnostic threshold of 0.9 was defined in these experiments, such that a ratio of Aβ42 percent labeled/Aβ40 percent labeled below 0.9 classified a subject as AD positive and a ratio of Aβ42 percent labeled/Aβ40 percent labeled above 0.9 classified a subject as AD negative. To determine whether the ratio of Aβ42 percent labeled/Aβ40 percent labeled at 23 hrs post infusion was differentiated between the three groups of patients, the ratio obtained for each patient was graphed versus PIB staining. As can be seen in FIG. 20A, a threshold of 0.9 for this ratio clearly differentiates the majority of MC+ subjects from the NC subjects (6/7 MC+ subjects were below the threshold, while 11/12 NC subjects were above the threshold). Within the MC− group, 3/4 of the subjects were below the threshold. It is possible, however, that subjects in the MC− group were in the early stages of AD. Similarly, the average of the 23 hour and 24 hour labeling percentages may be compared as a ratio between Aβ42 and Aβ40. Aβ42 percent labeled/Aβ40 percent labeled at 23 hrs post infusion and 24 hrs was differentiated between the three groups of patients, the ratio obtained for each patient was graphed versus PIB staining. As can be seen in FIG. 20B, with this measure, 7/7 MC+ subjects are below the threshold, while 11/12 NC are above the threshold. For the MC− group, 2/4 subjects are below the threshold.

These data may be compared to a simple measure that uses the results from the full kinetic model. In this case, the parameter kex42, which describes the rate of entry of Aβ42 into the exchange compartment, is multiplied by 10 and then added to the ratio of the rate constants for irreversible loss for Aβ42 versus Aβ40. As shown in FIG. 20C, a threshold of 1.75 shows that 6/7 of the MC+ subjects are above the threshold, with 12/12 of the NC subjects below the threshold. For the MC− group, 2/4 subjects are below the threshold.

These examples indicate that simple measures that summarize some aspect of the SILK tracer curve may be diagnostic of AD. This also indicates that short term collection of CSF may be sufficient to diagnose changes in Aβ42 kinetics.

### Example 10

### Introduction to Examples 11-16

The Aβ precursor protein (APP), produced in high amounts by neurons, is known to be degraded by different enzymes [2]. The enzyme β-secretase cleaves APP to produce the C99 peptide. C99 is then further processed by α-secretase to produce Aβ peptides of different lengths (e.g. Aβ38, Aβ40, Aβ42, where the number indicates the number of amino acids in the peptide). Aβ peptides are able to self-aggregate, with Aβ42 being more prone to formation of large aggregates [3], and the major constituent of senile plaques [4].

A promising approach to characterize the kinetics of Aβ production and clearance in humans relies on in vivo labeling of Aβ peptides during protein translation via infusion of stable isotope-labeled amino acids, stable isotope labeling kinetics (SILK) [5]. The fraction of isotope-labeled Aβ is measured at timed intervals in cerebrospinal fluid (CSF) collected at the lumbar subarachnoid space. The traditional method to estimate rates of irreversible loss of Aβ peptides from the CNS is analysis of the terminal slopes of isotopic enrichment time course curves evaluated on log-normal plots. This analysis method yields a measure that is referred to herein as the monoexponential fractional clearance rate (monoexponential FCR) [6]. Previous results demonstrated decreased monoexponential FCR of both Aβ40 and Aβ42 in late-onset AD [7]. However, the monoexponential FCR should not be confused with the true underlying fractional clearance rate, which may be difficult to determine in complicated systems. The true fractional clearance rate is the rate of irreversible loss of a product divided by the pool size of the product. To avoid confusion, the term fractional turnover rate or FTR is used herein, which has the same meaning as the true fractional clearance rate. The FTR is also equal to the sum of all of the rate constants describing routes of irreversible loss. The fractional synthesis rate (FSR) was determined by fitting a line to the upslope of the isotopic enrichment time course curve. FSR is defined as “the rate of incorporation from precursor to product divided by the pool size of the product”[6]. Thus, FSR is distinct from the production rate constant, which is the rate of incorporation from precursor to product divided by the pool size of the precursor. The FSR and monoexponential FCR analysis methods were acknowledged to have limitations, in that they imposed a simple one-compartment model on a complicated system [8]. However, more physiologically relevant models had not yet been developed.

Examples 1-8 introduced a physiologically relevant multi-compartmental model to distinguish carriers of presenilin-1 or presenilin-2 mutations that are the active enzymatic components of α-secretase and result in onset of AD at younger ages than non-mutation carriers (familial autosomal dominant AD). This work is also presented in detail in Sci. Transl. Med. 2013, pp. 189ra77, which is hereby incorporated by reference in its entirety. The main strength of the new model is that the rates of production, transport, reversible and irreversible loss of APP, C99, and the Aβ peptides may be estimated by fitting the model to the entire time course of the isotopic enrichment data while also accounting for the Aβ peptide concentrations in CSF. The model successfully detected an increase in the rate of production of Aβ42 relative to Aβ40 in human subjects with presenilin mutations, consistent with results in vitro and in mice [10]. Increased FTR of soluble Aβ42 relative to Aβ40 were also detected in participants known to have senile plaques demonstrated by positron emission tomography (PET) using Pittsburgh compound B (PIB). The previous observation of decreased monoexponential FCR of Aβ42 in late onset AD was re-interpreted in the context of amyloid positive mutation carriers when the full enrichment time courses were fit to the compartmental model [7]. From the analysis of Aβ isoforms in mutation carriers, it was concluded that the data actually reflected increased irreversible loss of soluble Aβ42 relative to Aβ40. Faster irreversible loss in combination with exchange of Aβ42 with higher order structures (e.g. aggregates, micelles, or the surface of pre-existing plaques) resulted in a ‘slower’ terminal exponential tail.

The compartmental model answered several questions concerning the amyloid hypothesis. However, the previous publication on the compartmental model did not discuss the identifiability of particular parameters[11] and [12]. In Examples 11-16, the identifiability of the different parameters in the compartmental model is described via a parameter sensitivity analysis. Analysis of the steady state of the model also revealed a potential mechanism for the decrease in the CSF concentration of Aβ42 in Alzheimer's disease [13].

Examples 11-16 refer to appendices A-K. Appendices A-K are Supplementary Materials to the publication entitled “Analysis of a compartmental model of amyloid beta production, irreversible loss and exchange in humans” (Mathematical Biosciences, 2015, pp. 48-61, Vol. 261). The publication and its Supplementary Material are incorporated herein by reference in their entirety.

### Example 11

### Methods and Theory/Calculation

Experimental methods for isotopic labeling of Aβ peptides and measurement of their concentrations in CSF are described in a separate publication [9]. Systems identifiability analysis and sensitivity analysis were performed as described in the text.

A compartmental model was constructed to describe Aβ peptide-labeling data FIG. 4 [9]. The brain was modeled as a reactor that produces APP from a pool of isotopically labeled plasma leucine with a zero-order rate constant k_{APP}. APP is then processed to become C99 (first order rate constant k_{C99}) or other products (first order rate constant v_{APP}). The production of other by-products from APP that impacts the production of C99, is governed by the product of the rate constant V_{APP} and the concentration of APP. C99 is further processed to produce soluble Aβ38, Aβ40, or Aβ42 and other products (e.g. Aβ peptides of other lengths) with first order rate constants k_{Ab38}, k_{Ab40}, k_{Ab42} and v_{C99}, respectively. Irreversible loss of each soluble Aβ peptide from the brain compartment that does not result in transport to CSF (e.g. insoluble deposition, degradation, or transfer across the blood-brain barrier) is modeled as first order processes with rate constants v₃₈, v₄₀, v₄₂ for the respective Aβ peptides. The soluble Aβ peptides may also enter a reversible, short-term exchange compartment while in the brain (kₑₓ₃₈, kₑₓ₄₀ and kₑₓ₄₂ for entry into and kᵣₑₜ₃₈, kᵣₑₜ₄₀ and kᵣₑₜ₄₂ for return from the respective compartments). Transport of soluble Aβ peptides out of the brain into the CSF is modeled as a first order process with rate constants k_{CSF38}, k_{CSF40} and k_{CSF42}, respectively. In practice, kᵣₑₜ₃₈, kᵣₑₜ₄₀ and kᵣₑₜ₄₂ were assumed to be identical and were called simply kᵣₑₜ and k_{CSF38}, k_{CSF40} and k_{CSF42} were assumed to be equal (with justifications to follow). Transport within the CSF is modeled by three compartments with equal first order exit rate constants (k_{delay} or sometimes k_{del}), which are assumed to be the same for all Aβ peptides. The lumbar CSF concentration and isotopic labeling of each Aβ peptide was measured and used in the model as the target concentration and labeling fraction in each peptide's third delay compartment. Appendix A describes the development of the model, starting from a mathematical model with the minimal structure necessary and sufficient to account for the shape of the isotopic enrichment time courses, and progressing through steps that transformed this starting model into a physiologically relevant model. The model in Appendix A was simplified compared to that shown in FIG. 4, due to empirical observation of identifiability issues for some of the parameters. To address identifiability concerns rigorously, the exact solution to the rate equations for the full model shown in FIG. 4 was calculated and is described below. A more detailed description of the exact solution is found in Appendix B.

### Example 11.1

### APP Labeling Kinetics During Infusion of Isotope-Labeled Leucine

Prior to the addition of labeled leucine, a steady state was presumed whereby the rate of production of the unlabeled protein (k_{APP}) was equal to the rate of conversion to C99 (−k_{C99}×c_{APP}) or other products (−v_{APP}×c_{APP}). A steady state pool size of APP (concentration of APP multiplied by compartment volume) was assumed throughout the labeling experiment, thus:

$$\begin{matrix}\frac{d {c}_{APP , SS}}{d t} = {k}_{APP} - ( {k}_{C 99} + {v}_{APP} ) {c}_{APP , SS} = 0 or , & Eqn . ( 11.1 .1 ) \\ {c}_{APP , SS} - \frac{{k}_{APP}}{{k}_{C 99} + {v}_{APP}} . & equation ( 11.1 .2 )\end{matrix}$$

To simplify the analysis, the fraction of isotopically labeled leucine in plasma was taken to be the average value during the labeling phase, f. Rates of change in the pool size of unlabeled APP (c_{APP}) and labeled APP (c_{APPL}) during the infusion phase are thus:

$$\begin{matrix}\frac{d {c}_{APP}}{d t} = {k}_{APP} ( 1 - f ) - ( {k}_{C 99} + {v}_{APP} ) {c}_{APP} and & Eqn . ( 11.1 .3 ) \\ \frac{d {c}_{APPL}}{d t} = {k}_{APP} f - ( {k}_{C 99} + {v}_{APP} ) {c}_{APPL} . & Eqn . ( 11.1 .4 )\end{matrix}$$

These equations imply that labeled leucine is added to tRNA proportional to the fraction f of labeled leucine, not the tracer-to-tracee ratio (TTR). The fraction f is [labeled leucine]/([unlabeled leucine]+[labeled leucine]), while the TTR is [labeled leucine]/[unlabeled leucine]. The fraction f has been shown to be the appropriate model for protein synthesis [15], and differs from the previous analysis method that used the TTR [7], although at the limit of low enrichment this is a minor difference. The use of TTR versus fractional labeling is further described in Appendix C.

The equations are solved:

$$\begin{matrix}{c}_{APP} = {c}_{APP 0} {e}^{- ( {k}_{C 99} + {v}_{APP} ) t} + \frac{{k}_{APP}}{( {k}_{C 99} - {v}_{APP} )} ( 1 - f ) ( 1 - {e}^{- ( {k}_{C 99} - {v}_{APP} ) t} ) and & Eqn . ( 11.1 .5 ) \\ {c}_{APPL} - {c}_{APPL 0} {e}^{- ( {k}_{C 99} + {v}_{APP} ) t} + \frac{{k}_{APP}}{( {k}_{C 99} + {v}_{APP} )} f ( 1 - {e}^{- ( {k}_{C 99} + {v}_{APP} ) t} ) . & Eqn . ( 11.1 .6 )\end{matrix}$$

The initial conditions at the moment of addition of labeled amino acid were:

$$\begin{matrix}{c}_{APP 0} = \frac{{k}_{APP}}{( {k}_{C 99} + {v}_{APP} )} and & Eqn . ( 11.1 .7 ) \\ {c}_{APPL 0} = 0. & Eqn . ( 11.1 .8 )\end{matrix}$$

The solutions appropriate for these initial conditions are:

$$\begin{matrix}{c}_{APP} = \frac{{k}_{APP}}{( {k}_{C 99} + {v}_{APP} )} ( 1 - f ( 1 - {e}^{- ( {k}_{C 99} + {v}_{APP} ) t} ) ) and & Eqn . ( 11.1 .9 ) \\ {c}_{APPL} = \frac{{k}_{APP}}{( {k}_{C 99} + {v}_{APP} )} f ( 1 - {e}^{- ( {k}_{C 99} + {v}_{APP} ) t} ) . & Eqn . ( 11.1 .10 )\end{matrix}$$

If the rates of production and irreversible loss of APP do not change during the course of the labeling experiment, the pool sizes of labeled plus unlabeled protein will equal the original steady state pool size of protein:

c_{APP, SS}=c_{APP}+c_{APPL} Eqn. (11.1.11).

Stated another way, the pool size of unlabeled protein must decline because a fraction of the tRNAs are loaded with the labeled amino acid.

The fractional labeling of APP (p_{APPL}) is obtained by dividing Eqn. (11.1.10) by Eqn. (11.1.2):

$${p}_{APPL} = \frac{{c}_{APPL}}{{c}_{APP , SS}} = \frac{{c}_{APPL}}{{k}_{APP} / ( {k}_{C 99} + {v}_{APP} )} = f ( 1 - {e}^{- ( {k}_{C 99} + {v}_{APP} ) t} ) . \qquad Eqn . ( 11.1 .12 )$$

The same result would be obtained by dividing the rate equation for c_{APPL} by the steady state concentration of APP and solving this differential equation:

$$\begin{matrix}\frac{d ( \frac{{c}_{APPL}}{{c}_{APP , SS}} )}{d t} = \frac{d {p}_{APPL}}{d t} = \frac{{k}_{APP} f - ( {k}_{C 99} + {v}_{APP} ) {c}_{APPL}}{{c}_{APP , SS}} \\ = \frac{{k}_{APP} f}{{k}_{APP} / ( {k}_{C 99} + {v}_{APP} )} - ( {k}_{C 99} + {v}_{APP} ) {p}_{APPL} \\ = ( {k}_{C 99} + {v}_{APP} ) ( f - {p}_{APPL} ) .\end{matrix} \qquad Eqn . ( 11.1 .13 )$$

Notice that the ‘rate of appearance’ of labeled APP in Eqn. (11.1.12) does not depend on the parameter k_{APP}. Basic kinetic intuition would suggest that the slope of the initial portion of the labeling curve should equal to the APP synthesis rate constant k_{APP}. This would be true if concentrations or pool sizes were measured, but not if TTR or fractional labeling is measured. To see this, the exponential terms in the equations for c_{APPL} and p_{APPL} are expanded as Taylor series in time. Assuming very short times, the terms in t² and higher may be neglected:

$${c}_{APPL} = \frac{{k}_{APP}}{( {k}_{C 99} + {v}_{APP} )} \times f ( 1 - ( 1 - ( {k}_{C 99} + {v}_{APP} ) t - \frac{1}{2} ( {( {k}_{C 99} + {v}_{APP} ) t )}^{2} + … ) ) ≃ {fk}_{APP} t . \qquad Eqn . ( 11.1 .14 )$$

However, for fractional labeling:

$${p}_{APPL} = f ( 1 - ( 1 - ( {k}_{C 99} + {v}_{APP} ) t + \frac{1}{2} {( ( {k}_{C 99} + {v}_{APP} ) t )}^{2} + … ) ) ≅ f ( {k}_{C 99} + {v}_{APP} ) t . \qquad Eqn . ( 11.1 .15 )$$

Although this section specifically described APP production and clearance rates, the conclusions are valid for any one compartment model. The initial slope of a labeling curve for a one-compartment model yields a measure of the irreversible loss rate constant and not its production rate constant. However, later it will be shown that the upslope of a labeling curve for a system described by a multicompartment model is more complicated.

### Example 11.2

### APP Labeling Kinetics Following Removal of Isotope-Labeled Leucine

At the end of the labeling period, the infusion of labeled amino acid ceases. The labeled fraction of isotope-labeled leucine in plasma drops rapidly and is well-described by a bi-exponential decay:

f=f₀(αe^{-q}ₘ^{t}+βe^{-q}ᵣ^{t}) Eqn. (11.2.1)

where f₀ is the fraction of labeled amino acid in plasma during the labeling period. For compactness, t=0 in this equation corresponds to the end of the labeling period. The sum of the parameters α and β is one. The parameters α and qₘ tend to be large and presumably represent rapid clearance of the labeled amino acid throughout the body. The parameters and qᵣ tend to be much smaller and likely represent reappearance of labeled leucine in plasma due to exchange of labeled plasma amino acid with non-plasma spaces and/or incorporation into and subsequent degradation of rapidly turning over proteins throughout the body.

At the end of the labeling period, labeled APP had a pool size of c_{APPL, end}. The rate equation for labeled APP Eqn. (11.1.4) is solved with the new expression for f and with initial condition c_{APPL}(0)=c_{APPL, end}:

$${c}_{APPL} = {k}_{APP} {f}_{0} ( \frac{α}{{k}_{C 99} + {v}_{APP} - {q}_{m}} {e}^{- {q}_{m} t} + \frac{β}{{k}_{C 99} + {v}_{APP} - {q}_{r}} {e}^{- {q}_{r} t} ) + ( {c}_{APPL , end} - {k}_{APP} {f}_{0} ( \frac{α}{{k}_{C 99} + {v}_{APP} - {q}_{m}} + \frac{β}{{k}_{C 99} + {v}_{APP} - {q}_{r}} ) ) \times {e}^{- ( {k}_{C 99} + {v}_{APP} ) t} . \qquad Eqn . ( 11.2 .2 )$$

The pool size of APP at the end of the labeling period is obtained from Eqn. (11.1.10):

$${c}_{APPL , end} = \frac{{k}_{APP}}{{k}_{C 99} + {v}_{APP}} f ( 1 - {e}^{- ( {k}_{C 99} + {v}_{APP} ) {t}_{end}} ) \qquad Eqn . ( 11.2 .3 )$$

where t_{end} is the length of the labeling period.

Dividing by the pool size of APP at steady state, the fractional labeling of APP after removal of labeled amino acid is:

$${p}_{APPL} = ( {k}_{C 99} + {v}_{APP} ) {f}_{0} ( \frac{α ( {e}^{- {q}_{m} t} - {e}^{- ( {k}_{C 99} + {v}_{APP} ) t} )}{{k}_{C 99} + {v}_{APP} - {q}_{m}} + \frac{β ( {e}^{- {q}_{r} t} - {e}^{- ( {k}_{C 99} + {v}_{APP} ) t} )}{{k}_{C 99} + {v}_{APP} - {q}_{r}} ) + {p}_{APPL , end} {e}^{- ( {k}_{C 99} + {v}_{APP} ) t} . \qquad Eqn . ( 11.2 .4 )$$

The first term on the right hand side represents new synthesis of labeled APP due to residual labeled amino acid. If the ‘new synthesis’ term is neglected, then a semilog-y plot would yield:

ln(p_{APPL})=ln(p_{APPL,end})−(k_{C99}±v_{APP})t Eqn. (11.2.5)

with slope of −(k_{C99}+v_{APP}). This illustrates the fact that the downslope of a one compartment model would yield an approximation of the rate constants describing ‘clearance’ but no information about rate constants of ‘production’ (the full model described below does not neglect new synthesis, unlike simple fits of monoexponential curves to the downslope of the labeling curve).

### Example 11.3

### Labeling Kinetics in Other Compartments

The rate equation that describes production of labeled C99 during the labeling phase is:

$$\frac{d {c}_{C 99 L}}{d t} = {k}_{C 99} {c}_{APPL} - ( {k}_{Ab 38} + {k}_{Ab 40} + {k}_{Ab 42} + {v}_{C 99} ) {c}_{C 99 L} . \qquad Eqn . ( 11.3 .1 )$$

The rate constants k_{Ab38}, k_{Ab40}, and k_{Ab42} govern the rate of production of Aβ38, Aβ40 and Aβ42, respectively. The rate constant v_{C99} describes all other irreversible losses of C99, including production of Aβ peptides of other molecular weights. For compactness, k_{Ab}=k_{Ab38}+k_{Ab40}+k_{Ab42}+v_{C99}.

The rate equations for all three Aβ peptides are similar and will be elaborated for Aβ42 only. The rate equation for labeling kinetics of soluble Aβ42 in the ‘brain’ is:

$$\frac{d {c}_{Ab 42 L}}{d t} = {k}_{Ab 42} {c}_{C 99 L} - ( {v}_{42} + {k}_{CSF} + {k}_{ex 42} ) {c}_{Ab 42 L} + {k}_{ret 42} {c}_{Ab 42 exL} . \qquad Eqn . ( 11.3 .2 )$$

The parameter k_{Ab42} represents production of Aβ42 from C99 by the action of α-secretase, v₄₂ describes irreversible loss of Aβ42 from the soluble brain compartment by means other than transfer to CSF, k_{CSF} describes irreversible loss into CSF, kₑₓ₄₂ describes entry into an exchange compartment, kᵣₑₜ₄₂ describes return of Aβ42 from the exchange compartment to the ‘brain’ compartment, and c_{Aβ42exL} is the pool size of labeled Aβ42 in the exchange compartment.

The kinetics of entry/exit of labeled Aβ42 into/from the exchange compartment is described by the rate equation:

$$\frac{d {c}_{Ab 42 exL}}{d t} = {k}_{ex 42} {c}_{Ab 42 L} - {k}_{ret 42} {c}_{Ab 42 exL} . \qquad Eqn . ( 11.3 .3 )$$

Without wishing to be bound by theory, it is believed that the ‘exchange’ compartment represents a reversible interaction with higher order structures, perhaps with the surface of amyloid plaques or oligomers (see reference [9] and Appendix A for additional discussion) [9]. In contrast, permanent or even slowly reversible assimilation into stable plaques would lead to an increase in the parameter v₄₂, because the labeled Aβ would not return to the soluble form during the time course of the experiment. This would thus be indistinguishable from other mechanisms of irreversible loss of Aβ42.

The rate equations for the three CSF delay compartments are:

1) First CSF delay compartment:

$$\frac{d {c}_{Ab 2 d 1 L}}{d t} = {k}_{CSF} {c}_{Ab 42 L} - {k}_{del} {c}_{Ab 42 d 1 L} \qquad Eqn . ( 11.3 .4 )$$

2) Second CSF delay compartment:

$$\frac{d {c}_{Ab 42 d 2 L}}{d t} = {k}_{del} ( {c}_{Ab 42 d 1 L} - {c}_{Ab 42 d 2 L} ) \qquad Eqn . ( 11.3 .5 )$$

3) Third CSF delay compartment:

$$\frac{d {c}_{Ab 42 d 3 L}}{d t} = {k}_{del} ( {c}_{Ab 42 d 2 L} - {c}_{Ab 42 d 3 L} ) . \qquad Eqn . ( 11.3 .6 )$$

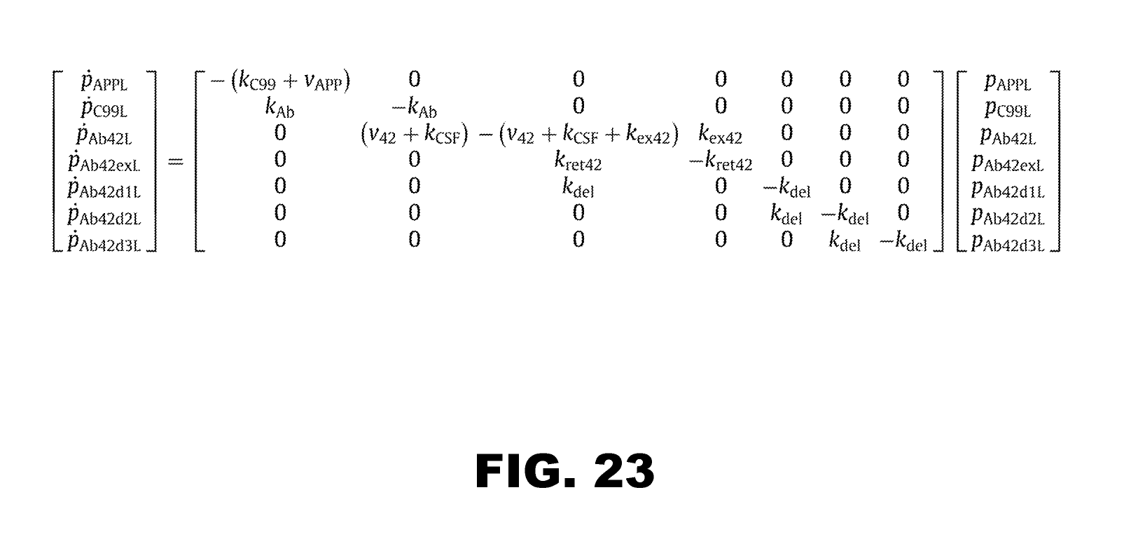

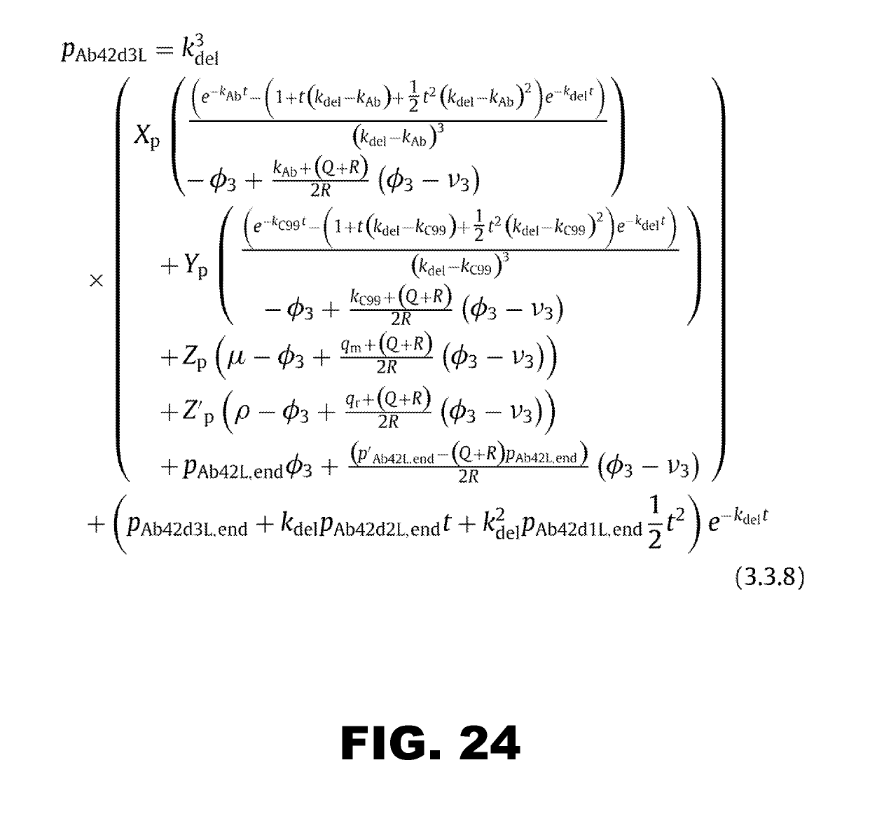

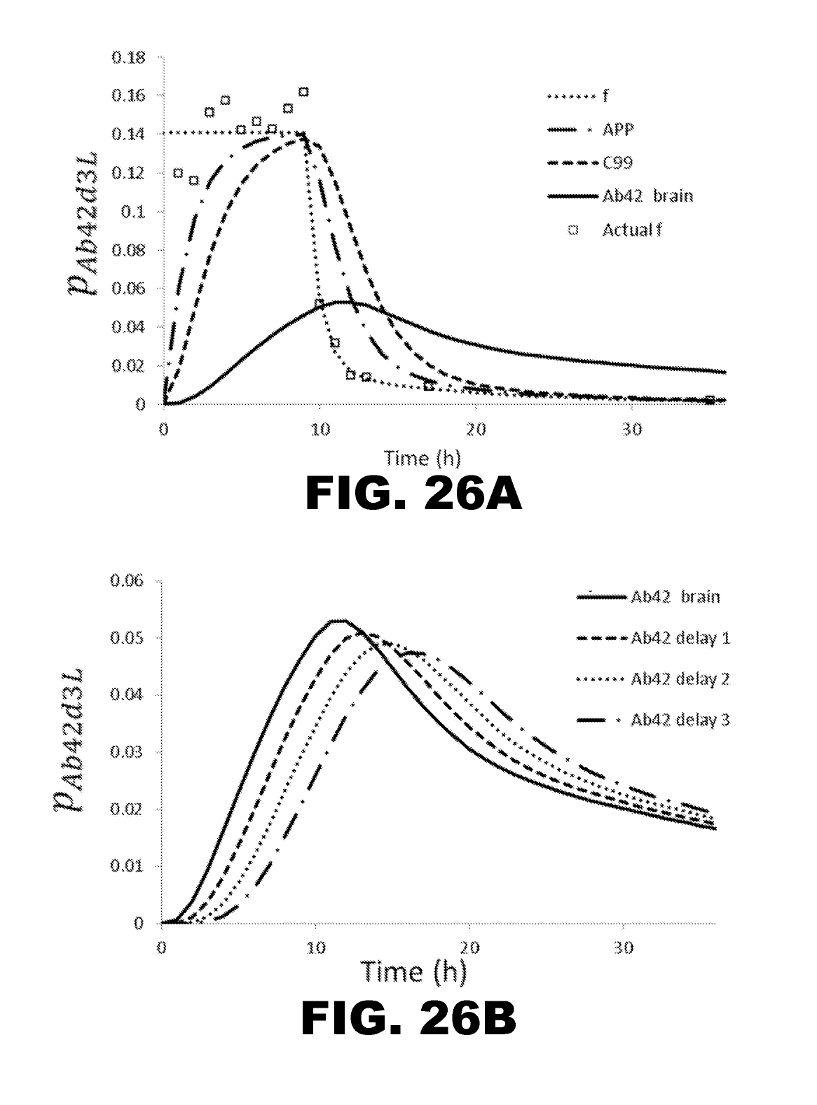

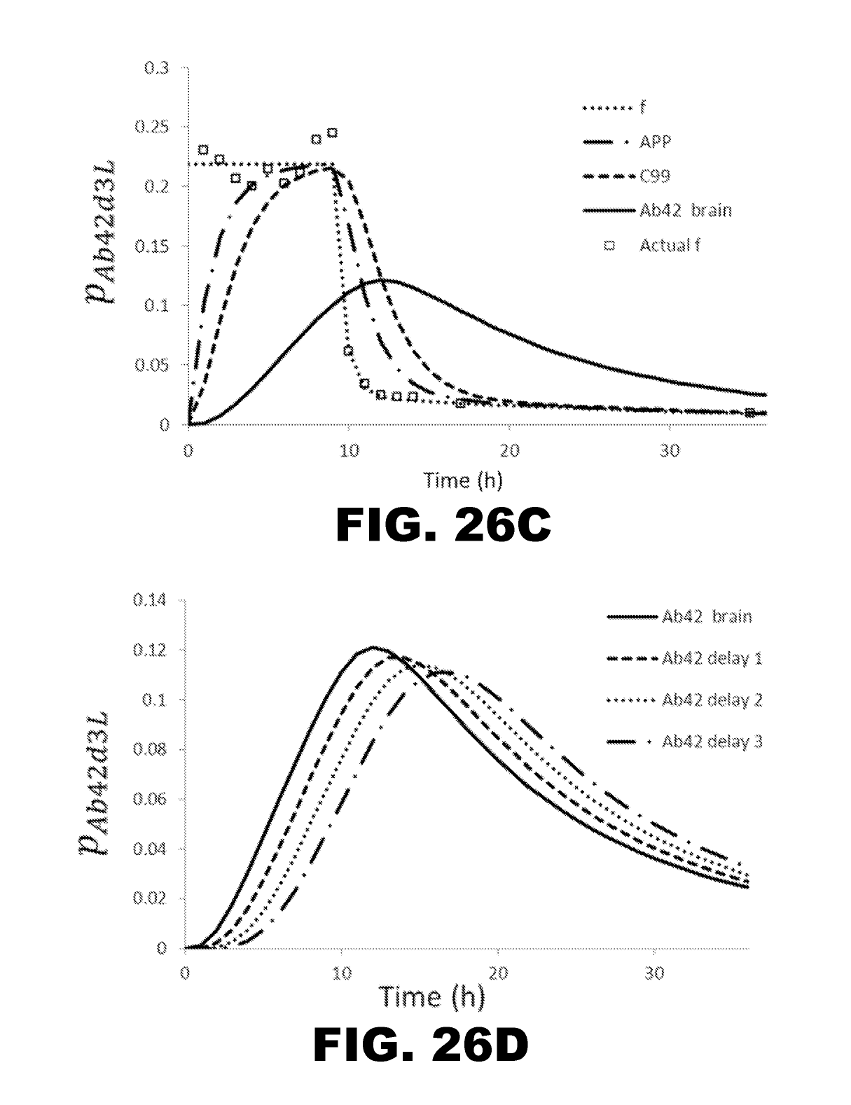

The system of differential equations in terms of fractional labeling may be written as Eqn (11.3.7), shown in FIG. 23, and may be solved directly. The solution during the post-labeling phase is Eqn. (11.3.8), shown in FIG. 24, with the full derivation shown in Appendix B, and solution with definitions of coefficients summarized in Appendix D for easy reference. For the labeling phase, the same equation applies but with f=f₀, meaning that qᵣ and qₘ in Eqn. (11.2.1) are equal to zero, and fractional labeling is zero at t=0 for all peptides. The predicted time course of labeling in each compartment is shown in FIG. 26.

Eqn. (11.3.8) describes the shape of the isotopic enrichment time course curve according to this compartmental model. An important conclusion is that the rate constant for production of Aβ42 (k_{Ab42}) does not appear in these equations except through its inclusion in k_{Ab}. Thus, any impact that k_{Ab42} has on Aβ labeling kinetics would only be manifest if this caused an increase in the rate of turnover of C99. If increases in secretase activity to produce Aβ42 are exactly balanced by decreases in production of other Aβ isoforms, then the model predicts that increases in the rate of production of Aβ42 would not be detectable by an isotope labeling experiment alone. However, as will be shown by a steady state analysis, the rate of production of Aβ peptides may be calculated by using both Aβ42 isotope labeling kinetics and CSF Aβ42 concentration data.

### Example 11.4

### Fractional Synthesis Rate (FSR) and Fractional Clearance Rate (FCR) in Multicompartment Systems

The goal of the experimental studies was to determine the rate constants for the production (k_{Ab38}, k_{Ab40} and k_{Ab42}) and irreversible loss (v₃₈, v₄₀ and v₄₂) of the Aβ peptides. In the former case, this may sometimes be stated as determining the ‘production rates of the Aβ peptides’. Because the Aβ peptides have a common precursor (C99), the production rate constants are in fact the true determinants of the production rates. Similarly, it may be stated that the ‘clearance rates’ are of interest. However, this is much less precise, because these rates (or fluxes, both with units of mass/time or concentration/time) depend on the pool size/concentration of each Aβ peptide, which differ greatly. In fact, the kinetic measures that allow meaningful comparisons of irreversible loss between the different Aβ isoforms are the ‘clearance rate constants’ or ‘irreversible loss rate constants’.

Because the models are at steady state, the production rate and irreversible loss rate must be equal. Thus, only one rate is required, the ‘turnover’ rate [16] and [17]. The turnover rate divided by the concentration or pool size of the product is the fractional turnover rate (FTR), which is equal to the irreversible loss rate constant. The ‘fractional synthesis rate’ is the rate of appearance of labeled product divided by the pool size or concentration of the product [6], which is the same as dividing the turnover rate by the product pool size. Thus, the fractional synthesis rate is theoretically the same as the FTR (i.e. true FCR) and the irreversible loss rate constant. However, the ‘FSR’ often refers to the method of estimating the fractional turnover rate by fitting a line to the upslope of a curve and dividing the slope by the enrichment of the precursor. This method of estimating FTR is only accurate for systems well-described by single-compartment models. However, CSF Aβ kinetics are best described by a multi-compartmental model, and the ‘FSR’ that was previously applied to CSF Aβ kinetics [7] and [14] may thus actually reflect changes in the production rate constant, one of the two quantities of interest.

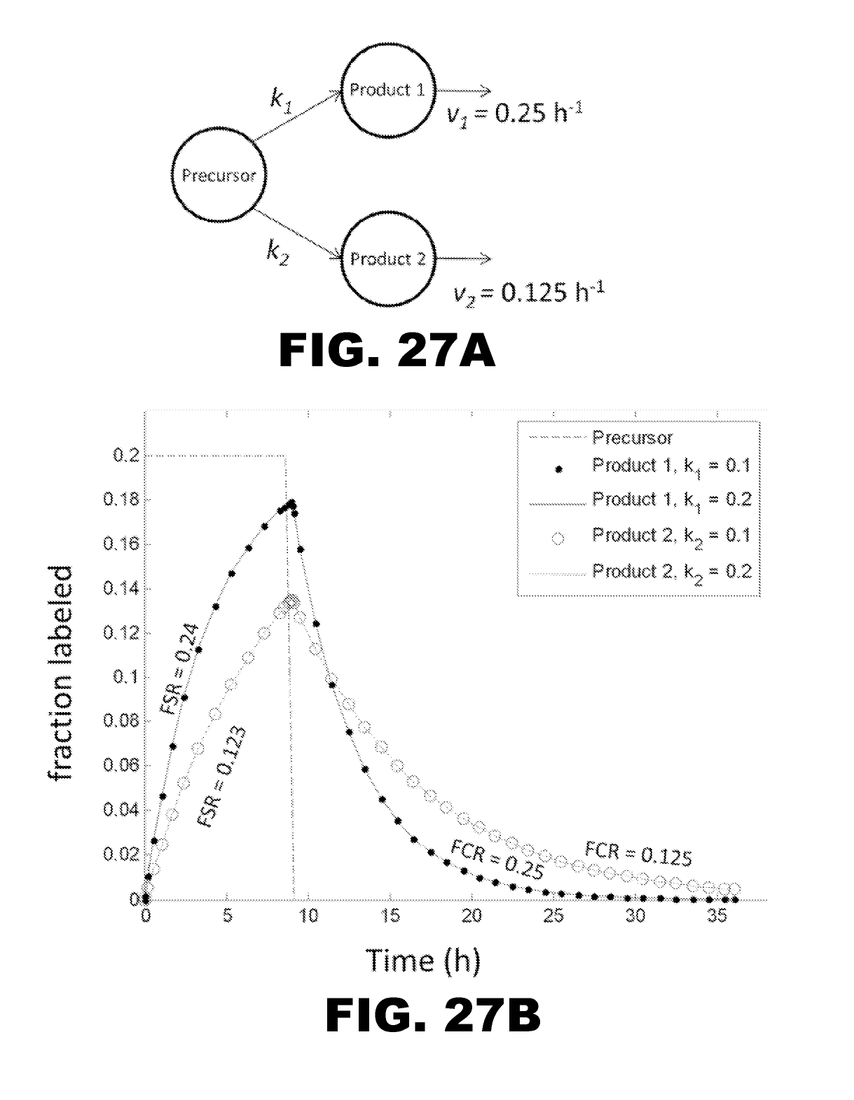

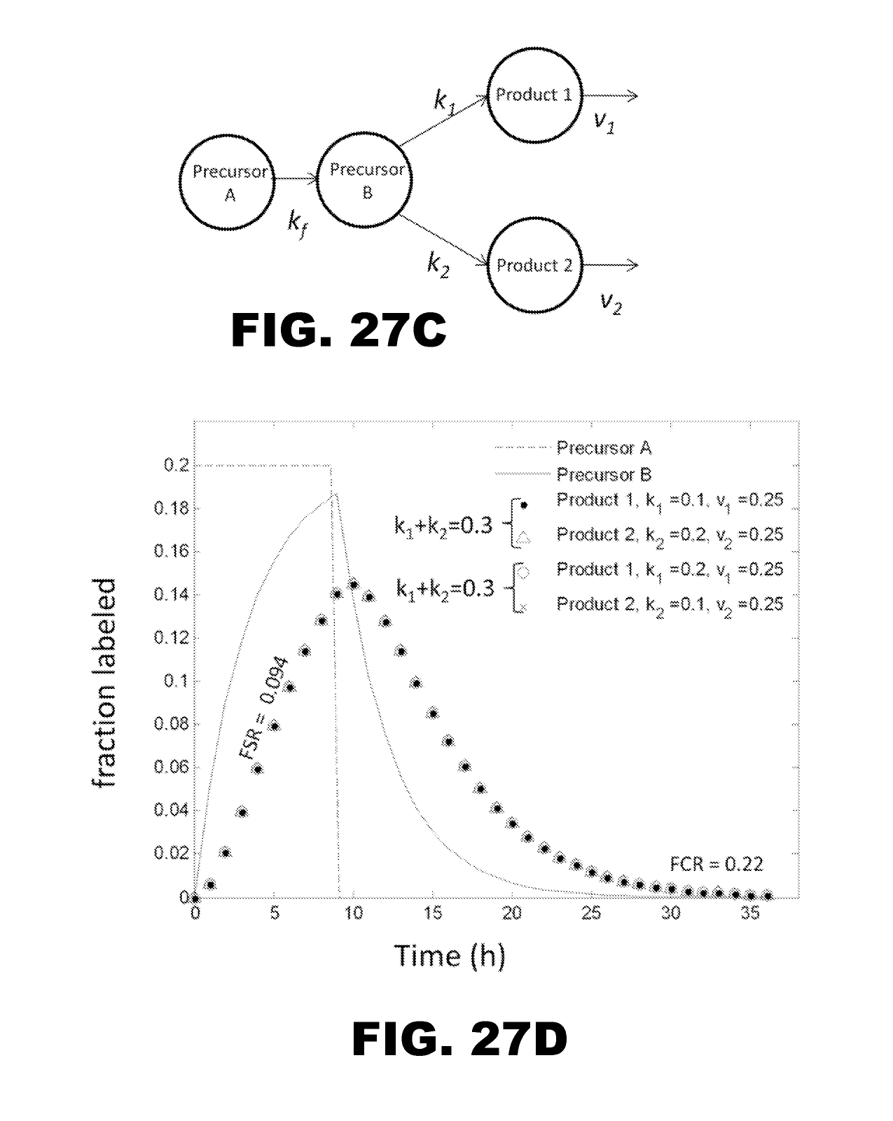

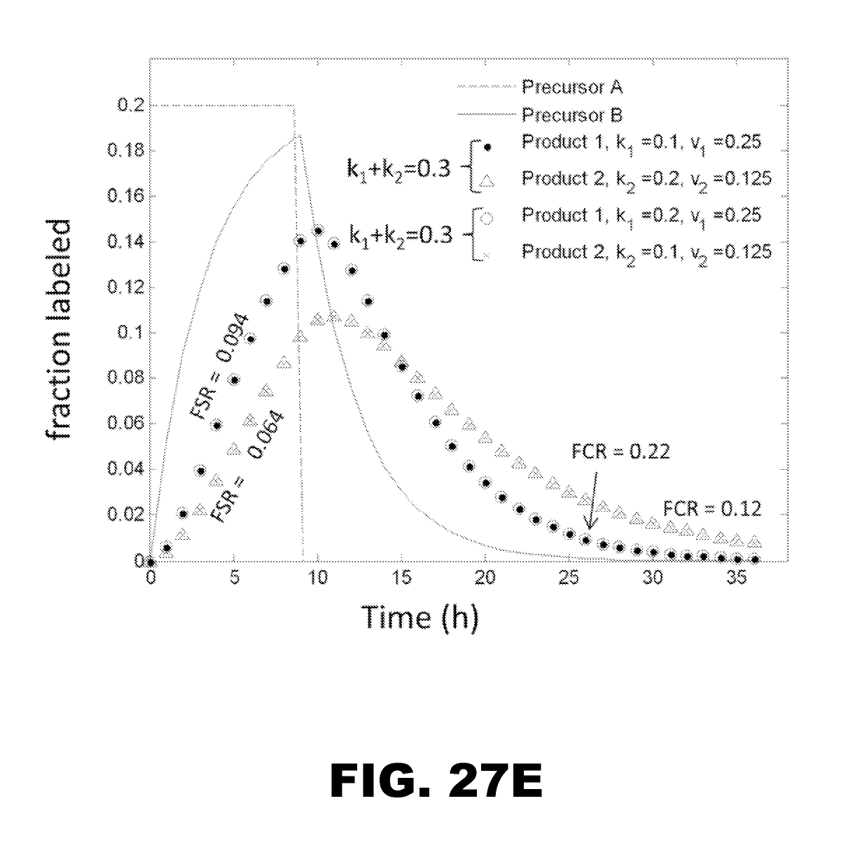

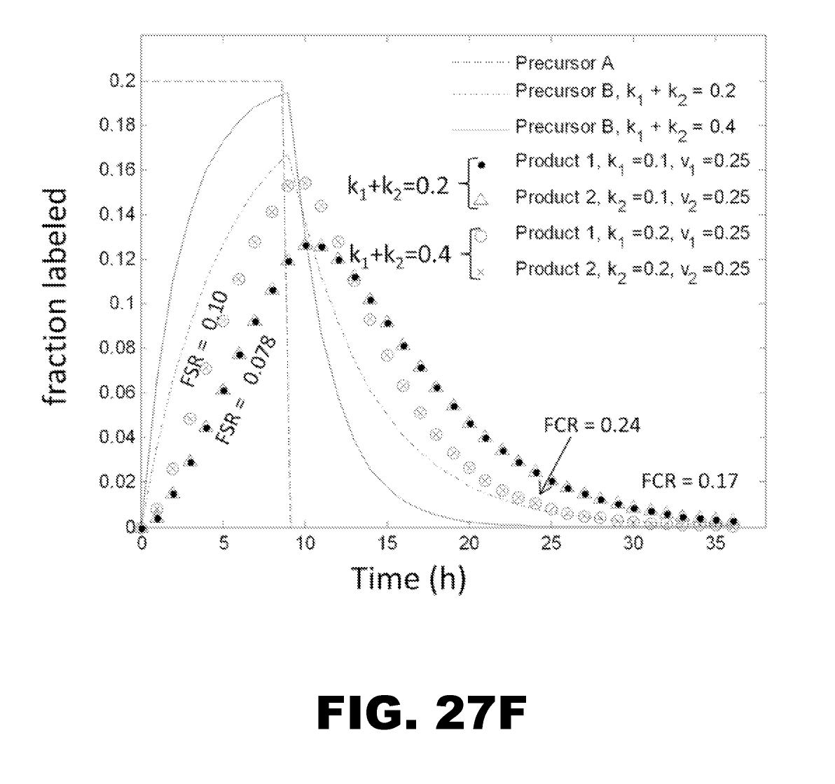

FIG. 27 illustrates these concepts. A simple model is simulated (FIG. 27A), in which a precursor with constant concentration during a 9-h labeling phase may produce two products with different irreversible loss rate constants (v₁ and v₂). However, FIG. 27A is simply two parallel one-compartment models. The production rate constants (k₁ and k₂) are varied. The FSR is estimated from the initial slope of the product labeling curves (first three data points), while the FCR is the monoexponential slope from 24 to 36 h (FIG. 27B). As the production rate constants vary, the labeling curves do not change in the one-compartment models. However, both the FSR and FCR provide good estimates of the fractional turnover rate (i.e. irreversible loss rate constants v₁ and v₂). The production rate constants are:

$$\begin{matrix}\begin{matrix}{k}_{1} = \frac{{v}_{1} \times {c}_{product 1.55}}{{c}_{precursor , ss}} \\ = \frac{{FCR}_{product 1} \times {c}_{product 1 , ss}}{{c}_{{ }_{precursor , ss}}} \\ = \frac{{FSR}_{product 1} \times {c}_{product 1 , ss}}{{c}_{precursor , ss}}\end{matrix} & Eqn . ( 11.4 .1 ) \\ \begin{matrix}{k}_{2} = \frac{{v}_{2} \times {c}_{product 2 , ss}}{{c}_{precursor , ss}} \\ = {FCR}_{product 2} \times \frac{{c}_{product 2 , 55}}{{c}_{precursor , ss}} \\ = \frac{{FSR}_{product 2} \times {c}_{product 2 , ss}}{{c}_{precursor , ss}}\end{matrix} & Eqn . ( 11.4 .2 )\end{matrix}$$

where c_{productx, SS} is the steady state concentration of product x.

In FIG. 27C, the model is expanded into a multi-compartmental model, where precursor A is at a constant concentration during the labeling phase, and produces precursor B, which then splits to produces products 1 and 2. The rate constant k_{f} has no impact on the labeling curve, but could be calculated as:

$${k}_{f} = \frac{( {k}_{1} + {k}_{2} ) \times {c}_{precursor B , ss}}{{c}_{precursorA , ss}} . \qquad Eqn . ( 11.4 .3 )$$

Changes in k₁ or k₂ also do not impact the labeling curve as long as +k₂ remains constant (FIG. 27D). At constant k₁+k₂, the shape of the labeling curve is only affected by v₁ and v₂ (FIG. 27E). However, if k₁+k₂ varies, the labeling curve shape is affected (FIG. 27F). Thus, k₁+k₂ is identifiable, but k₁ and k₂ are unidentifiable [11]. At constant v₁ and v₂, increases in k₁+k₂ result in higher values for FSR and FCR, with the FCR coming closer to v₁ or v₂ (FIG. 27F). The meaning of the measured value of the FSR for multicompartment systems is difficult to decipher, however it is clear that FSR is a measure of both production and irreversible loss, but only if an increase in production of the product causes a change in the irreversible loss of its precursor. Similar to the one-compartment model, the production rate constant is easily calculated if the irreversible loss rate constant is multiplied by the pool size/concentration of the product.

### Example 11.5

### Steady State Analysis

In addition to measurement of the fractional labeling of each of the Aβ peptides in the CSF, the concentration of each peptide in the CSF was measured by mass spectrometry. The concentrations of the Aβ peptides in CSF provided additional constraints on the parameters in the system. Although some diurnal variation in Aβ42 concentration in the CSF has been noted [18], the concentration in CSF at the start of the experiment was assumed to represent a steady state throughout the experiment.

To calculate pool size in each CSF compartment, the measured CSF concentration was multiplied by a typical CSF volume of 135 mL, and divided by 3 to account for three equal-volume CSF compartments in the model. The assumption that every participant had a CSF volume of 135 mL divided into three compartments seems strong but actually has little impact on the results. If the CSF concentrations of Aβ peptides were used instead of pool sizes, the results for all of the first-order rate constants would be identical, but the zero-order rate constant k_{APP} would simply be lower by a factor of 3/135. Because k_{APP} does not affect the shape of the predicted isotope-labeling curve, use of either concentrations or pool sizes in fitting the labeling curves is justified.

According to the current model, the pool size of Aβ42 measured in the lumbar CSF is equal to the steady state pool size of Aβ42 in the third delay compartment (see Eqn. 11.3.5 and 11.3.6). The steady state pool sizes of Aβ42 in each of the three delay compartments must be equal:

C_{Ab42,delay3,SS}=C_{Ab42,delay2,SS}=C_{Ab42,delay1,SS} Eqn. (11.5.1).

Relative to the pool size of soluble Aβ42 in the brain, the pool size of Aβ42 in each delay compartment is predicted to be scaled by a factor k_{CSF}/k_{del},

$${c}_{Ab 42 , delay 3 , ss} = \frac{{k}_{CSF}}{{k}_{del}} {c}_{Ab 42 , brain , ss} . \qquad Eqn . ( 11.5 .2 )$$

The exchange compartment has no effect on the steady state pool size of soluble Aβ peptides in the brain or CSF. However, the pool size of the exchange compartment itself is:

$${c}_{Ab 42 , exchange , ss} = \frac{{k}_{ex 42}}{{k}_{rep 42}} {c}_{Ab 42 , brain , ss} . \qquad Eqn . ( 11.5 .3 )$$

Deposition into plaques or aggregates that do not return labeled Aβ42 on the time scale of the experiment would only impact the irreversible loss parameter, v₄₂. Thus, rates of deposition of Aβ42 into plaques can be estimated by comparing the difference between v₄₂ and v₄₀, or between v₄₂ and v₃₈, because Aβ38 and Aβ40 deposition into plaques is expected to be minimal [19].

After additional substitutions for the steady state concentrations of APP, C99, and Aβ peptides in the brain, the steady state concentrations in the CSF for each of the Aβ peptides is predicted to be:

$$\begin{matrix}{c}_{Ab 38 , delay 3 , ss} = \frac{{k}_{CSF} {k}_{c 99} {k}_{APP}}{{k}_{del} ( {k}_{c 99} + {v}_{APP} )} \times \frac{{k}_{Ab 38}}{( {v}_{38} + {k}_{CSF} ) ( {k}_{Ab 38} + {k}_{Ab 40} + {k}_{Ab 42} + {v}_{c 99} )} & Eqn . ( 11.5 .4 ) \\ {c}_{Ab 40 , delay 3 , ss} = \frac{{k}_{CSF} {k}_{c 99} {k}_{APP}}{{k}_{del} ( {k}_{c 99} + {v}_{APP} )} \times \frac{{k}_{Ab 40}}{( {v}_{40} + {k}_{CSF} ) ( {k}_{ab 38} + {k}_{Ab 40} + {k}_{Ab 42} + {v}_{c 99} )} & Eqn . ( 11.5 .5 ) \\ {c}_{Ab 42 , delay 3 , ss} = \frac{{k}_{CSF} {k}_{c 99} {k}_{APP}}{{k}_{del} ( {k}_{C 99} + {v}_{APP} )} \times \frac{{k}_{Ab 42}}{( {v}_{42} + {k}_{CSF} ) ( {k}_{Ab 38} + {k}_{Ab 40} + {k}_{Ab 42} + {v}_{C 99} )} . & Eqn . ( 11.5 .6 )\end{matrix}$$

Overall, the model has 25 parameters:

k_{APP}, v_{APP}, k_{C99}, c_{C99}, k_{Ab38}, k_{Ab40}, k_{Ab42}, V₃₃, v₄₀, v₄₂, kₑₓ₃₈, kₑₓ₄₀, kₑₓ₄₂, kᵣₑₜ₃₈, kᵣₑₜ₄₀, kᵣₑₜ₄₂, k_{CSF38}, k_{CSF40}, k_{CSF42}, k_{del38}, k_{del40}, k_{del42}, SF₃₈, SF₄₀, SF₄₂
The last three parameters are scaling factors that were applied to the predicted labeling curve for each peptide. The scaling factors were found to improve the fit and may correct for systematic errors caused by variability in the standard curves used in the daily calibration of the mass spectrometers, or isotopic dilution between plasma leucine and APP production. The mean values of the scaling factors for all participants were 0.941±0.08, 0.944±0.08, and 0.937±0.11 for Aβ38, Aβ40 and Aβ42, respectively, with no significant differences between groups. The model predictions of fractional labeling of the Aβ peptides in the CSF are linearly related to the scaling factors, and thus sensitivity to this parameter in isolation is uninformative.

As will be shown below, this model is ‘system unidentifiable’. To reduce the number of parameters, the following assumptions were applied:

k_{CSF}=k_{CSF38}=k_{CSF40}=k_{CSF42}

k_{del}=k_{del38}=k_{del40}=k_{del42}

kᵣₑₜ=kᵣₑₜ₃₈=kᵣₑₜ₄₀=kᵣₑₜ₄₂.

The first two parameters (k_{CSF} and k_{del}) represent fluid flow processes and likely affect all three peptides equally. The third parameter (kᵣₑₜ) could only be discerned for Aβ42 (see Appendix A) and may be different for Aβ38 and Aβ40. However, choosing the same value for kᵣₑₜ for all three peptides allowed us to examine the extent of exchange of Aβ38 and Aβ40 relative to Aβ42. Exchange of Aβ38 and Aβ40 was found to be minimal and improved the fit of the model to the data in only a few subjects.

These assumptions reduced the model to 19 parameters. The CSF concentration size of each peptide is known, and because of steady state relationships Eqn. (11.5.4), Eqn. (11.5.5) and Eqn. (11.5.6), only 16 of the 19 parameters are independent. The choice of which three parameters are considered to be dependent is arbitrary, but the Aβ production rate constants k_{Ab38}, k_{Ab40} and k_{Ab42} are easily calculated (see Appendix E) and a convenient choice.

### Example 11.6

### Simplified Model

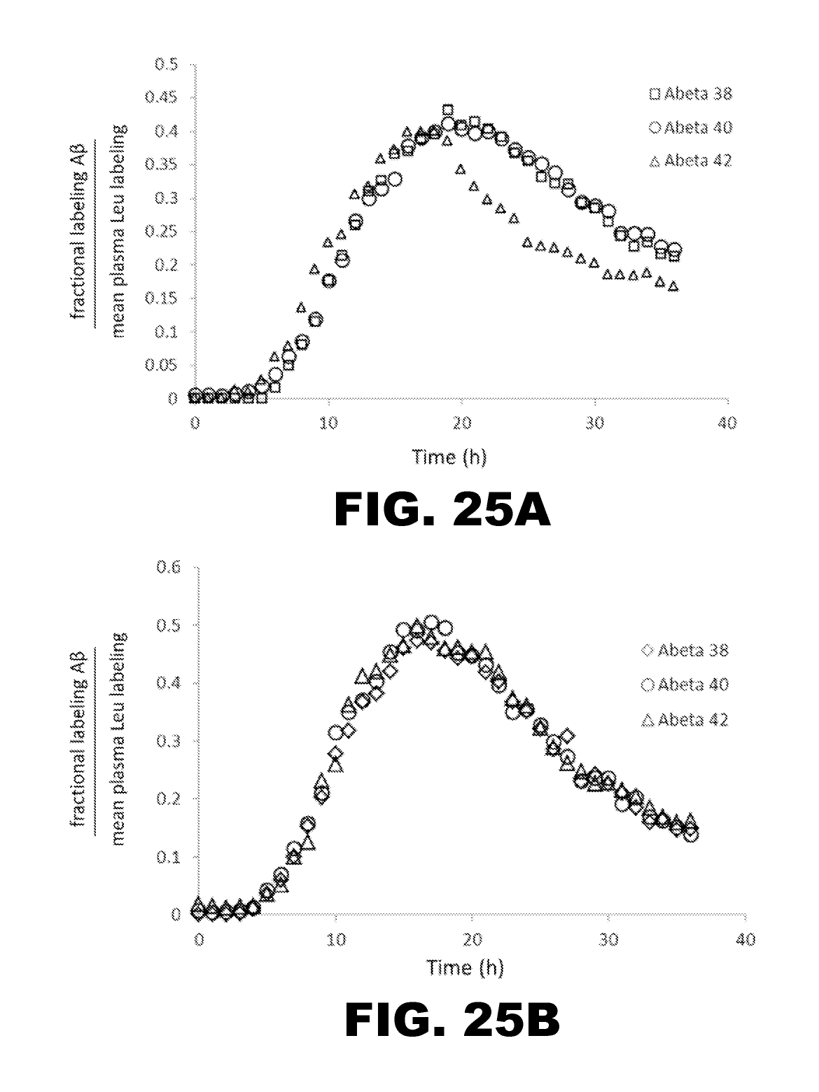

Most of the Aβ isotope-labeling curves were found to be well-fit by a simple model consisting of five ‘delay’ compartments arranged in series, with equal-valued rate constants for transfer between compartments, plus a single compartment turning over at a unique rate (see Appendix A). The data sets that could not be fit were primarily Aβ42 in subjects with significant amyloid plaque load as demonstrated by PET-PIB. The different morphology of the Aβ42 isotopic labeling time course compared to Aβ38 and Aβ40 in PIB− positive subjects is readily observed (e.g. see FIG. 25A). The Aβ42 isotopic labeling time course from PIB− positive subjects was only well-fit when an exchange compartment was added to the model.

Although the exact solution presented above incorporates known biology and physiology, the current dataset was unable to independently identify all 16 rate constants in the model, for reasons that will be clear following the system identifiability and sensitivity analysis below. Thus, the model was further simplified using the following assumptions:

$${k}_{C 99} = {k}_{del}$$

$${v}_{APP} = 0$$

$${v}_{C 99} = \frac{1}{2} {k}_{del}$$

$${k}_{Ab} = {k}_{Ab 38} + {k}_{Ab 40} + {k}_{Ab 42} + {v}_{C 99} = {k}_{del}$$

$${v}_{40} = {k}_{CSF}$$

$${k}_{ret} = 0.1 {h}^{- 1}$$

Justifications for these assumptions are driven by the need to replace some of the poorly identified rate constants (i.e. k_{C99} and k_{Ab}) with k_{del}, thus producing a model that was quite similar to a simple five compartment delay that was known to be sufficient to fit the labeling curves in subjects without plaques (Appendix A). The irreversible loss rate constant of APP was poorly identified (v_{APP}) and its effects were lumped into v_{C99}. It was further assumed that only half of C99 led to the production of Aβ38, Aβ40 and Aβ42. This is because Aβ peptides of other sizes are produced, with their abundance very roughly estimated from MALDI-TOF spectra of Aβ peptides in CSF [20]. It was further assumed that 50% of the irreversible loss of Aβ40 was to the CSF (i.e. v₄₀=k_{CSF}). Varying this fraction lost to the CSF between 10% and 90% had little effect on the results of the model (Appendix A). Finally, the return rate constant from the exchange compartment (kᵣₑₜ) was set to 0.1 h⁻¹. This was optimized using the three participants with the largest extent of Aβ42 exchange, using different fixed values of kᵣₑₜ and determining which value gave the best fit to the labeling curves (Appendix A). The six imposed relationships reduced the total number of parameters from 19 to 14 (because k_{C99}, v_{C99} and v₄₀ were replaced by other parameters and v_{APP} and kᵣₑₜ were set to specific values) and the number of independent parameters was reduced to 10 (an additional degree of freedom was lost by setting k_{Ab38}+k_{Ab40}+k_{Ab42}=½k_{del}). Choice of the four dependent parameters is arbitrary, but calculation of k_{APP}, k_{Ab38}, k_{Ab40} and k_{Ab42} from the other 10 parameters is illustrated in Appendix F. Exact solutions for the simplified model used in the previous publication are shown in Appendix G.

### Example 12

### System Identifiability

Although development of the simplified model was described in Example 11.6, the process was empirical. Using system identifiability analysis, a more rigorous approach is described here. The three transfer functions for the full model reveal that in principle 13 independent parameters may be determined from the labeling curve of each peptide (for methods, see references [11] and [12] and Appendix H). The full model has 25 parameters, demonstrating that the system is underdetermined. The assumptions from Example 11.5 of a common k_{CSF}, kᵣₑₜ and k_{del} for the three peptides were physiologically based and reduced the number of parameters to 19. The following parameters appear together as sums everywhere within the transfer functions: v₃₈+k_{CSF}, v₄₀+k_{CSF}, v₄₂+k_{CSF}, k_{C99}+v_{APP}, and k_{Ab38}+k_{Ab40}+k_{Ab42}+v_{C99}. This led to some of the assumptions of the simplified model, namely that k_{CSF} is a constant fraction of v₄₀, and that v_{APP} is zero. Additionally, if the SUM k_{Ab38}+k_{Ab40}+k_{Ab42}+v_{C99} is replaced with the one parameter k_{Ab}, the number of parameters is reduced to 14. Recognizing that k_{APP} does not appear in the rate equations for fractional labeling reduces the number of parameters to 13. Thus, these assumptions make the problem ‘system identifiable’ [11]. The 24 algebraic equations that appear in the transfer functions were not further manipulated to demonstrate ‘parameter identifiability’ due to their complexity. Rather, ‘practical identifiability’ issues with the model are demonstrated by the sensitivity analysis below, further motivating the reduction from 13 to 10 parameters in the simplified model.

### Example 13

### Sensitivity Analysis

Sensitivity analysis of the simplified model would not yield information about k_{C99} and k_{Ab} because these were explicitly replaced by k_{del} in the solution. Thus, the exact solutions to the full model were utilized in the sensitivity analysis. Sensitivity analysis was performed using parameters from a PIB− negative non-carrier and a PIB− positive presenilin-1 mutation carrier. Both participants were of similar age. The parameter values for the simplified model were originally optimized using the measured hourly plasma leucine enrichment data as the input. To simplify the sensitivity analysis, all of the parameters in the simplified model were re-optimized using the mathematical functions ƒ (Eqn. (11.2.1)) to describe plasma leucine values. The differences between the raw hourly plasma leucine data and the ƒ functions are shown in FIGS. 26A and C. The results of the parameter re-optimization are summarized in Appendix I.

The sensitivity analysis describes the sensitivity of the fractional labeling of Aβ42 in the third CSF compartment (p_{Aβ42d3L}) to changes in each of the major model parameters. For example, for k_{Ab42}, the sensitivity S_{kAb42} is:

$${S}_{kAb 42} = \frac{\partial {p}_{Ab 42 d 3 L}}{\partial {k}_{Ab 42}} . \qquad Eqn . ( 13.1 )$$

This is obtained by taking the partial derivative of the exact solution for p_{Ab42d3L} Eqn. (11.3.8) with respect to k_{Ab42}. The sensitivity can be interpreted as:

$$Δ {p}_{Ab 42 d 3 L} \approx Δ {k}_{Ab 42} \times \frac{\partial {p}_{Ab 42 d3L}}{\partial {k}_{Ab 42}} \qquad Eqn . ( 13.2 )$$

for small Δk_{Ab42}.

For the sensitivity analysis, the exact solutions become unbounded when k_{C99}→k_{del} or k_{Ab}→k_{del}. To overcome this, the derivatives with respect to each of the parameters was taken and then the limit of the resulting equations was evaluated as k_{Ab}→k_{C99} and then k_{C99}→k_{del}, applying L'Hôpital's rule when necessary. The detailed methods are described in Appendix J.

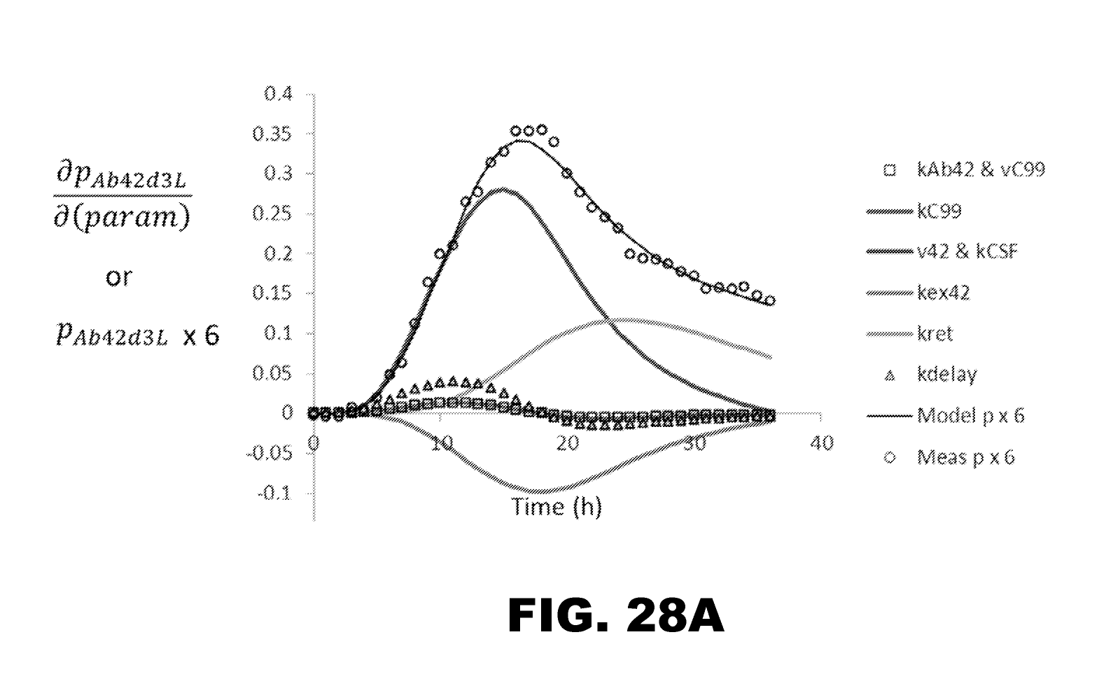

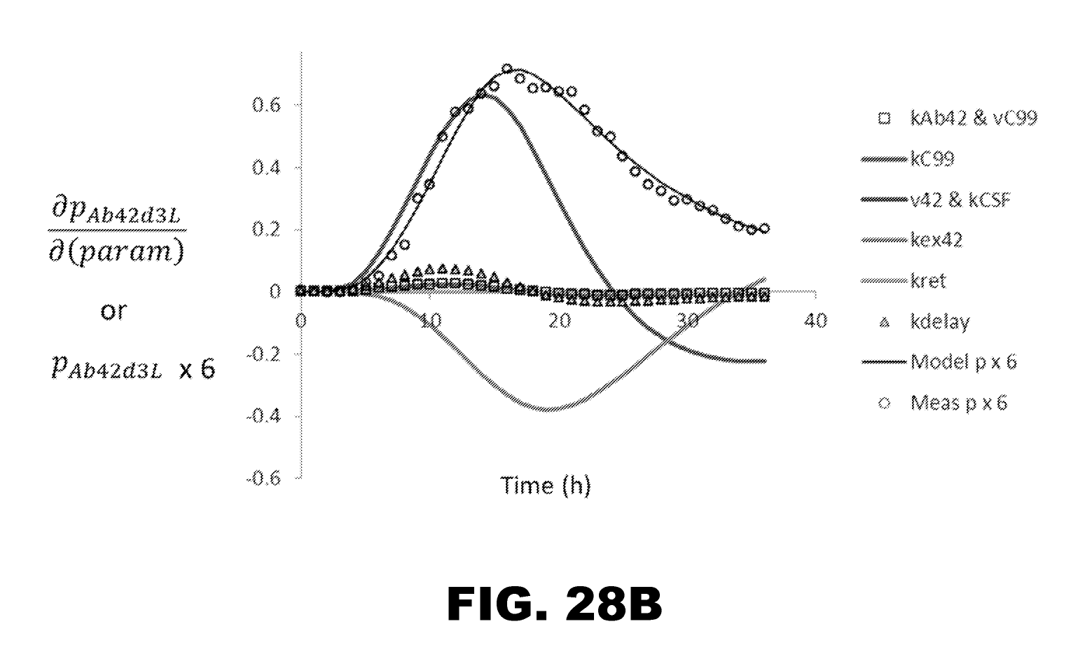

FIGS. 28A and B shows the sensitivity of p_{Ab42d3L} to the various parameters, along with the measured and model p_{Ab42d3L} (scaled by 6 for readability). The largest effect on p_{Ab42d3L} was found with changes in v₄₂. Identical sensitivity was observed for k_{CSF}, because both rate constants describe irreversible loss of Aβ42 (see Eqn. (11.3.2)). Within the first 5 h of labeling, increases in v₄₂ or k_{CSF} had no effect on p_{Ab42d3L}. This is expected, because of the delay in the appearance of Aβ42 in the final compartment. However, between hours 5 and 36, increased v₄₂ or k_{CSF} leads to increases in the values of p_{Ab42d3L} for the mutation carrier, with a maximum effect immediately prior to the peak enrichment of Aβ42.

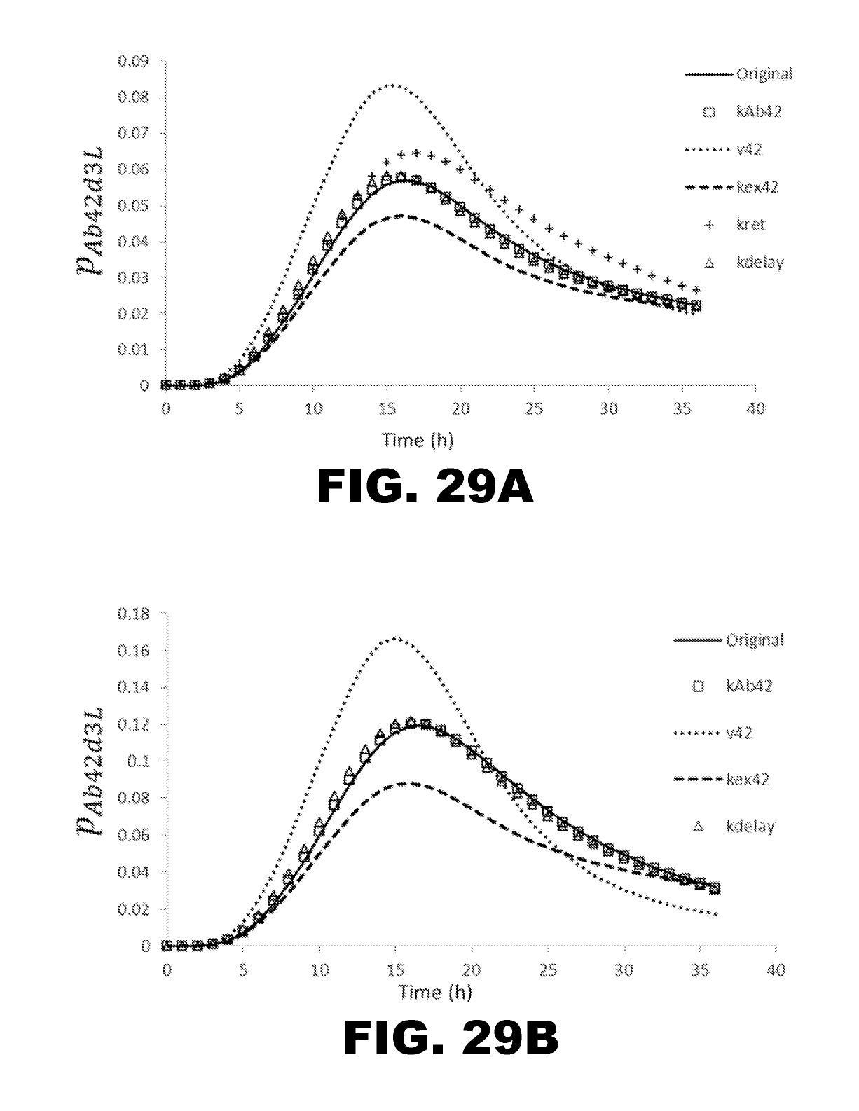

For the non-carrier, increases in v₄₂ or k_{CSF} also increased p_{Ab42d3L} between hours 5 and 24, with a maximum effect about 2 h prior to the peak enrichment of Aβ42. However, increases in v₄₂ or k_{CSF} decreased p_{Ab42d3L} between hours 24 and 36. The effects of increases in v₄₂ or k_{CSF} on actual kinetic curves are shown in FIGS. 29A and B (for these figures, the rate equations were solved numerically, increasing one of the parameter values by 0.1 h⁻¹ while holding all other parameters constant). Increasing v₄₂ results in the labeling curve rising earlier, peaking higher, and falling more quickly. However, in the mutation carrier (FIG. 29A), the quicker fall is halted after about 28 h, likely due to the effects of the exchange compartment.

Returning to FIG. 28, the next most important parameter that affected p_{Ab42d3L} was kₑₓ₄₂, the rate constant for entry of Aβ42 into the exchange compartment. An increase in this parameter lowered the peak p_{Ab42d3L} and flattened the tail of the curve in both participants (FIGS. 29A and B). Increasing the rate constant for exit of Aβ42 from the exchange compartment (kᵣₑₜ) lead to increase in p_{Ab42d3L} for the mutation carrier (FIG. 28A and FIG. 29A), but this only became substantial after the peak in Aβ42 enrichment. As expected, kᵣₑₜ had no effect with the non-carrier because no exchange was present in this participant (kₑₓ₄₂=0). The other parameters had only small effects on p_{Ab42d3L}, including k_{C99}, k_{Ab42}, k_{CSF} and k_{del} (k_{Ab42} and v_{C99} have identical sensitivities because both are constituents of k_{Ab}, which governs the irreversible loss of C99). Changes in the rate of irreversible loss of APP/C99 thus have much less of an effect on the Aβ42 labeling curve than the rate of irreversible loss of Aβ42 itself. Thus, substantial differences in labeling curves between subjects most likely reflect changes in the irreversible loss of Aβ42 and/or the presence of short term exchange, assuming that anatomical differences can be neglected.

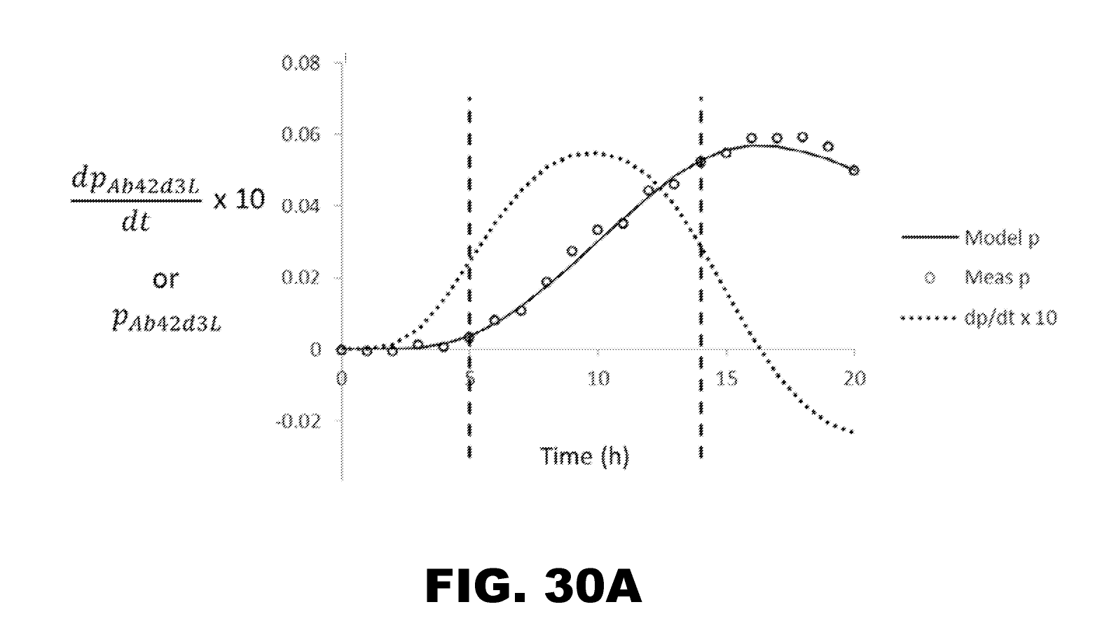

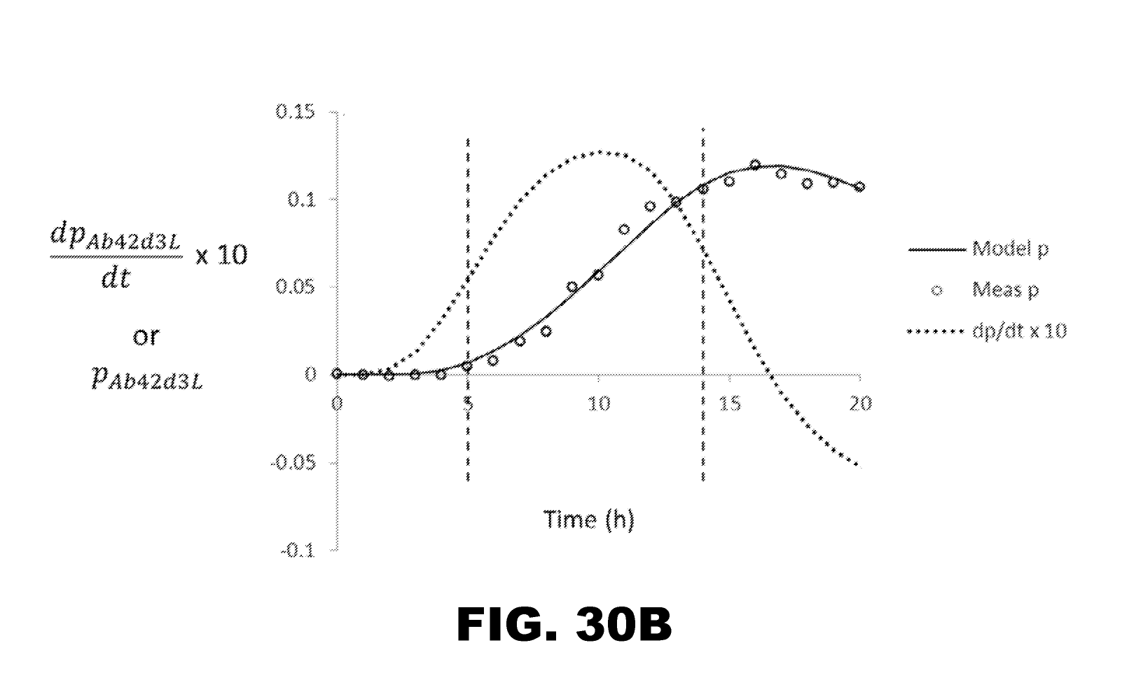

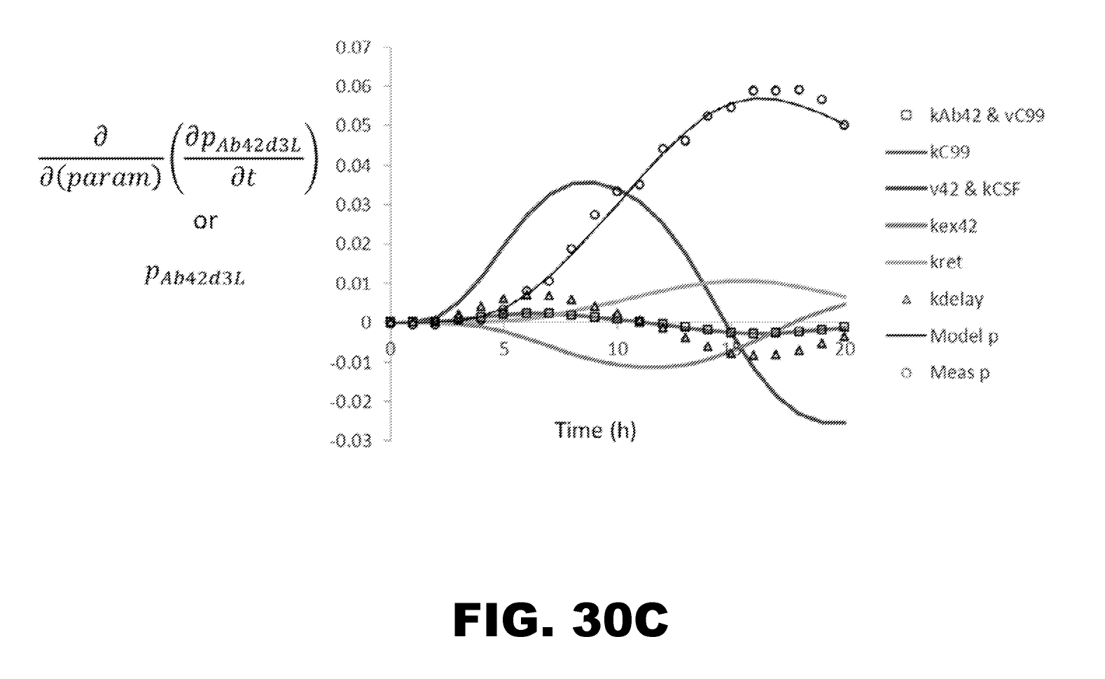

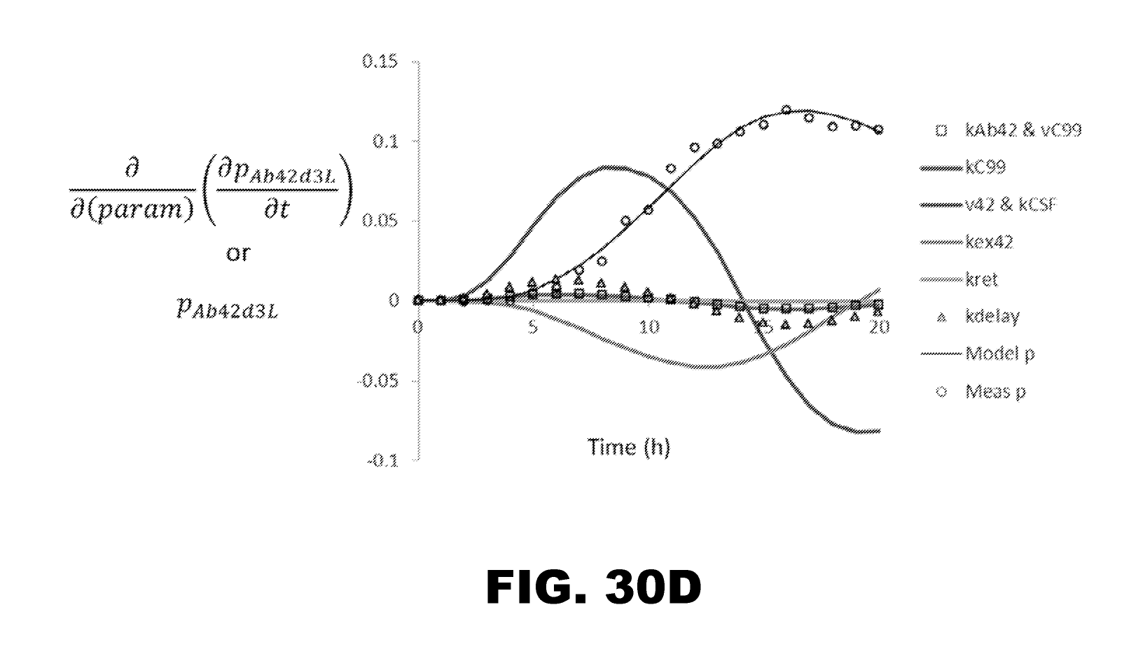

The sensitivity of the FSR to parameter changes in the model parameters was also examined (FIG. 30), which is simply the sensitivity of the time derivative of p_{Ab42d3L} (i.e. the slope of the labeling curve). Using the parameter k_{Ab42} as an example, this is:

$$\frac{\partial }{\partial {k}_{Al : 42}} ( \frac{\partial {p}_{At 42 d 3 L}}{\partial t} ) = \frac{{\partial }^{2} {p}_{Ab 42 d 3 L}}{\partial {k}_{Ab 42} \partial t} = \frac{\partial }{\partial t} ( \frac{\partial {p}_{Ab 42 d3L}}{\partial {k}_{Ab 42}} ) = \frac{\partial {S}_{kAb 42}}{\partial t} . \qquad Eqn . ( 13.3 )$$

FIGS. 30A and B shows the actual value of ∂p_{Ab42d3L}/∂t around the upslope of the labeling enrichment curve (scaled by 10 for readability). The value of ∂p_{Ab42d3L}/at varies considerably between 5 and 14 h, and resembles the result of fitting the middle portion of a sigmoidal curve to a straight line. FIGS. 30C and D shows the sensitivity of ∂p_{Ab42d3L}/∂t to changes in different parameters, and the measured p_{Ab42d3L} and model p_{Ab42d3L} in the region of the upslope are shown on all plots.

For both participants, the largest effect on ∂p_{Ab42d3L}/∂t (and thus FSR) came from v₄₂ and k_{CSF}. The next largest effect on FSR was from kₑₓ₄₂, which had an opposite effect from v₄₂ and k_{CSF}. Thus, if both of these parameters are increased (as was noted in participants with plaques), they will tend to cancel each other out. The parameter kᵣₑₜ had a modest effect on FSR, while the other parameters had even less effect.

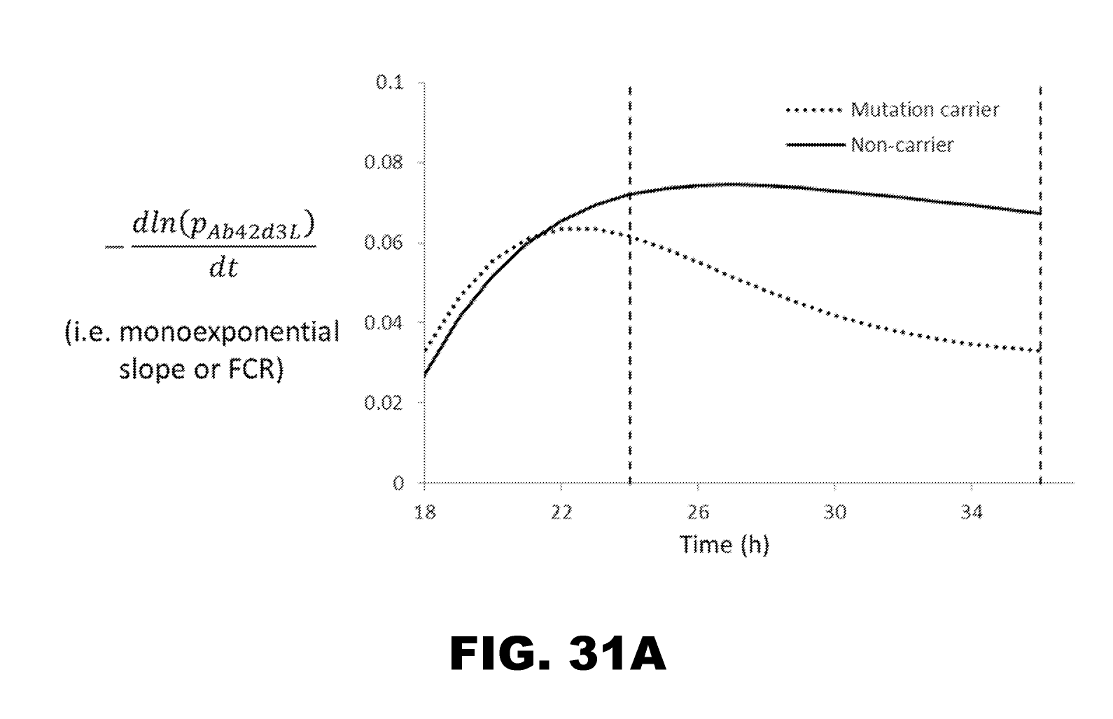

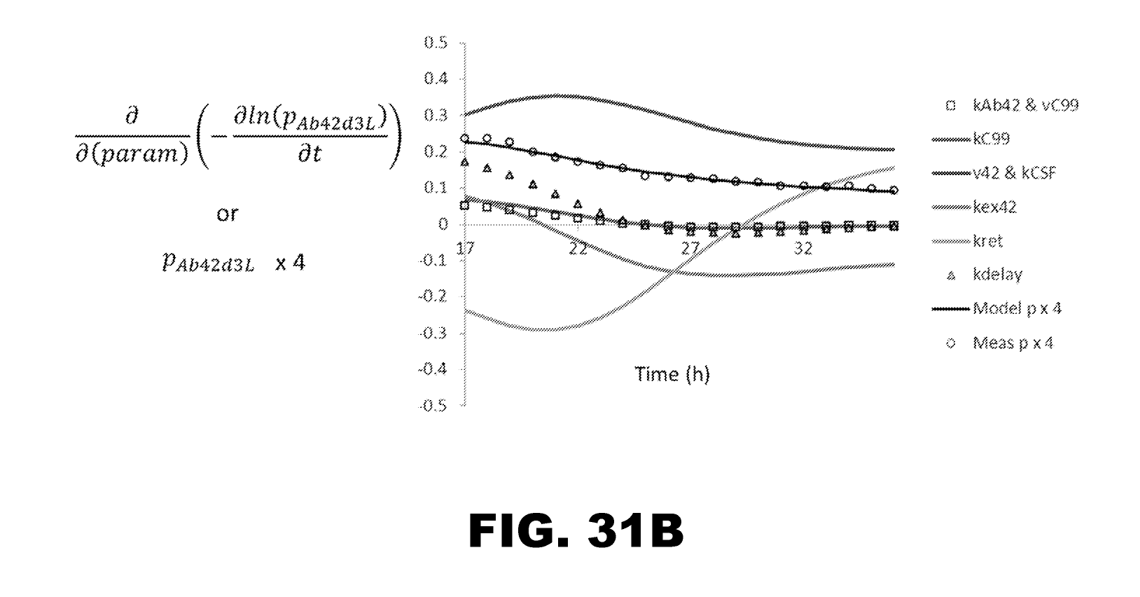

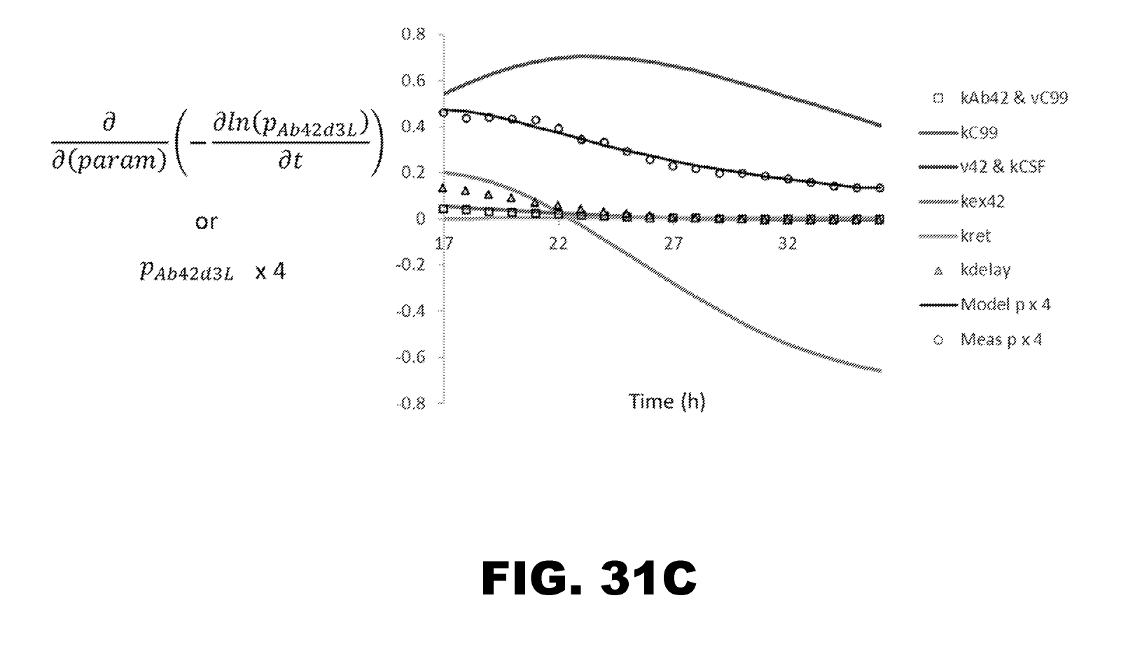

The sensitivity of the monoexponential FCR was calculated (FIG. 31), which is simply the sensitivity of the time derivative of the natural logarithm of p_{Ab42d3L}

$$\begin{matrix}\frac{d {S}_{kAb 42}^{log}}{d t} = \frac{d}{d {k}_{Ab 42}} ( \frac{d ln ( {p}_{Ab 42 d 3 L} )}{d t} ) \\ = \frac{d}{d {k}_{Ab 42}} ( \frac{d ln ( {p}_{Ab 42 d 3 L} )}{d {p}_{Ab 42 d 3 L}} \frac{d {p}_{Ab 42 d 3 L}}{d t} ) \\ = \frac{d}{d {k}_{Ab 42}} ( \frac{1}{{p}_{Ab 42 d 3 L}} \frac{d {p}_{Ab 42 d 3 L}}{d t} ) .\end{matrix} \qquad Eqn . ( 13.4 )$$

In FIG. 31A, the actual −∂ ln(p)/∂t for each participant is plotted. When −∂ ln(p)/∂t is relatively flat, this indicates a good monoexponential fit. For the non-carrier, −∂ ln(p)/∂t was relatively flat between 24 and 36 h, the exact region used previously to determine the monoexponential FCR [7]. For the mutation carrier with plaques, however, the curve is not flat, meaning that it would not be fit as well by a monoexponential function. Overall, −∂ ln(p)/∂t has a smaller mean value for the mutation carrier with plaques compared to the non-carrier, suggesting (incorrectly) decreased ‘clearance’ (i.e. irreversible loss) of Aβ42 in the mutation carrier with plaques compared to the normal control, when in fact irreversible loss is increased but masked by exchange.

The sensitivity of −∂ ln(p)/∂t to changes in parameters is presented in FIGS. 31B and C, along with the measured p_{Ab42d3L} and model p_{Ab42d3L} scaled by 4 for readability. The sensitivity analysis on a log scale shows that increase in v₄₂ or k_{CSF} lead to increase in monoexponential FCR (i.e. increases in −∂ ln(p)/∂t between 24 and 36 h), while increases in kₑₓ₄₂ would result in a decreased monoexponential FCR. The parameter kᵣₑₜ had a complicated effect on −∂ ln(p)/∂t, decreasing monoexponential FCR up to 30 h, but increasing it after that. The parameters k_{Ab42}, v_{C99} and k_{C99} had nearly negligible effects on monoexponential FCR.

The goal of the isotope-labeling study was to determine k_{Ab42}, which governs the production rate of Aβ42, and (v₄₂+k_{CSF}), which govern the irreversible loss rate of Aβ42. The sensitivity analysis demonstrated that most of the variation in the Aβ42 labeling curve between subjects is likely due to differences in v₄₂, k_{CSF}, kₑₓ₄₂ and kᵣₑₜ. However, k_{Ab42} may be reliably estimated because it has a large and direct effect on the concentration of Aβ42 in CSF. The sensitivity of the CSF Aβ42 concentration is the derivative of Eqn. (11.5.6) with respect to the various parameters. For example, for k_{Ab42}:

$${S}_{kAb 12}^{conc} = \frac{\partial {c}_{Ab 42 d 3 L}}{\partial {k}_{Ab 42}} = \frac{{k}_{CSF} {k}_{C 99} {k}_{APP}}{{k}_{del} ( {k}_{C 99} + {v}_{APF} ) ( {v}_{42} + {k}_{CSF} )} ( \frac{1}{{k}_{Ab}} - \frac{{k}_{Ab 42}}{{k}_{Ab}^{2}} ) . \qquad Eqn . ( 13.5 )$$

The sensitivity of Aβ42 CSF concentration to changes in the different parameters is presented in Table 10. The most important parameters that evoke changes in the CSF concentration of Aβ42 are k_{Ab42}, v₄₂ and k_{CSF}. The production rate constant of Aβ42 from C99 (k_{Ab42}) was the most important parameter in determining the CSF concentration in the non-carrier, and second only to k_{CSF} in the mutation carrier. Increases in k_{Ab42} or k_{CSF} are predicted to result in increases in the CSF concentration of Aβ42, whereas an increase in v₄₂ causes a reduction in CSF Aβ42 concentration because v₄₂ represents shunting of Aβ42 away from the CSF. For this reason, the model predicts that CSF Aβ42 concentration is decreased due to shunting to irreversible loss, perhaps including deposition into plaques. Thus, most of the information about the rate of production of Aβ42 is provided by the concentration of Aβ42 in CSF, while the shape of the isotopic enrichment curve tends to provide information about irreversible loss and exchange of Aβ42.

TABLE 10
Sensitivity of CSF concentrations of
Aβ42 to changes in listed parameters.

| S^{conc} with | Mutation- | Non- |
|---|---|---|
| respect to: | carrier | carrier |
| k_{APP} | 0.024 | 0.032 |
| v_{APP} | −0.83 | −1.4 |
| k_{C99} | 0 | 0 |
| v_{C99} | −0.83 | −1.4 |
| k_{Aβ38} | −0.83 | −1.4 |
| k_{Aβ40} | −0.83 | −1.395 |
| k_{Aβ42} | 12.7 | 30.7 |
| v₃₈ | 0 | 0 |
| v₄₀ | 0 | 0 |
| v₄₂ | −4.7 | −9.6 |
| k_{delay} | −0.83 | −1.4 |
| k_{CSF} | 14.3 | 8.8 |
| kₑₓ₃₈ | 0 | 0 |
| kₑₓ₄₀ | 0 | 0 |
| kₑₓ₄₂ | 0 | 0 |
| kᵣₑₜ | 0 | 0 |

### Example 14

### Effects of Scaling Factors and Baseline Correlation

Sensitivity analysis is not helpful to analyze the effects of the scaling factors. However, the scaling factors affect the overall size of the fitted curve, which allows other parameters to be adjusted in combination to better fit different regions of the curve. Examining FIG. 28, it is easy to imagine how changes in different parameter could reshape different parts of the curve. In Appendix K, the effects of removing the scaling factors are examined for both subjects. The parameters v₃₈, v₄₀, v₄₂, k_{CSF} appear to move in opposite directions from k_{C99}, v_{C99}, k_{Ab38}, k_{Ab40} and k_{delay}. In the mutation carrier with plaques, when the scaling factor is removed, the first group of parameters is increased and the second group is decreased. The opposite occurs in the non-mutation carrier, probably because this subject had a scaling factor less than one, while the mutation carrier had a scaling factor greater than one. Interestingly, the production rate constant k_{Ab42} was increased in both subjects when the scaling factor was removed. The effects of baseline correction were also studied. The baseline was considered to be the first five time points, and their average was subtracted from all data points. Removing the baseline correction improved the fit for the non-mutation carrier only. Overall, the scaling factors might be needed due to instrument calibration errors, isotopic dilution or the presence of other processes not well-captured by the current model.

### Example 15

### Relationship Between Production Rate Constants, Irreversible Loss Rate Constants, and CSF Concentration

The ratio of production rate constants for Aβ42 relative to Aβ40 is simply Eqn. (11.5.6) divided Eqn. (11.5.5):

$$\frac{{k}_{Ab 42}}{{k}_{Ab 40}} = \frac{{[ A {β}_{42} ]}_{CSF}}{{[ A {β}_{40} ]}_{CSF}} ( \frac{{v}_{42} + {k}_{CSF}}{{v}_{40} + {k}_{CSF}} ) . \qquad Eqn . ( 15.1 )$$

This shows that if the CSF concentration ratio of Aβ42:Aβ40 is to remain constant, increases in irreversible loss of Aβ42 relative to Aβ40 must be accompanied by increases in production of Aβ42 relative to Aβ40. However, if production is held constant and irreversible loss of Aβ42 relative to Aβ40 increases, as may occur in the presence of plaques, then the CSF concentration of Aβ42 relative to Aβ40 will decline, as has been observed [13]. This equation also shows that an increase in production without an increase in irreversible loss (perhaps due to an absence of plaques) should result in an increase in CSF concentration of Aβ42 relative to Aβ40. This has also been observed in mutation carriers that are much younger than their expected age of onset [13]. An important observation is that exchange of Aβ42 has no impact on the steady state CSF concentration, because the flux of mass into the exchange compartment is identical to the flux of mass out if at a steady state.

### Example 16

### Discussion of Examples 11-14

The sensitivity analysis demonstrated that the overall shape of the Aβ labeling curves was affected by all of the parameters in the model, although some parameters had much larger effects than others. Previously, the FSR of the labeling curve between 5 and 14 hours was used to estimate production kinetics of Aβ peptides [7]. The sensitivity analysis demonstrates that the Aβ isotopic enrichment upslope is not highly affected by differences in production rate constants between subjects. Rather, the FSR likely reflected primarily irreversible loss and exchange, although no differences in FSR were found between Alzheimer's subjects and controls. However, in this region of the labeling curves, increased irreversible loss and increased exchange will tend to act in opposite directions, potentially canceling out each other's effects on FSR. On the other hand, as expected, the monoexponential FCR is strongly affected by the rate of Aβ irreversible loss in the absence of short-term exchange. However, the presence of exchange complicates the use of monoexponential FCR as a reliable measure of the true turnover rate. Much more information about the system is gleaned by fitting the entire time course to the new compartmental model, which is rooted in the biology and physiology of the system. In addition, the simultaneous use of CSF concentrations along with the labeling data allows determination of rate constants for both production and irreversible loss of Aβ peptides.

In other models, it was suggested that only the first 15 h of labeling data were required to fully describe the kinetics of the system [14]. While it is possible that the irreversible loss rate might be reasonably well estimated from the upslope of the labeling curve in normal participants, the current sensitivity analysis shows that the presence of exchange will affect the upslope of the curve, potentially muting the effects of increased irreversible loss (FIGS. 30C and D). FIG. 25A illustrates that, in the mutation carrier, the largest difference between the Aβ40 and Aβ42 labeling curves occurs in the time period between about 19 and 30 h. Within this time frame, the effects of increased irreversible loss are declining, while the effects of exchange peak at about 19 h (FIGS. 28A and B). The sensitivity curve for v42 is not a perfect mirror image of that for kex42 and thus analyzing the full time course is the best hope for separating out the effects of irreversible loss and exchange.

The FSR has also been used to analyze the effects of α-secretase inhibitors on the labeling of Aβ peptides in humans and non-human primates [21] and [22]. Large changes in the upslope of the labeling curves were noted. This is not inconsistent with the present analysis. Although the sensitivity of the FSR to changes in production rate constants is small, it is not zero. In the case of inhibition of α-secretase, this should result in a large decrease in the production rate constants, resulting in a decrease in the FSR. As illustrated in FIG. 27, FSR is in fact a measure of production, although it is affected by other parameters as well. The transient introduction of the α-secretase inhibitors results in a non-steady state system, although the importance of the non-steady state nature of the system is difficult to estimate.

Several caveats about the compartmental model must be mentioned. Flow processes likely dictate the rate at which Aβ peptides transit from brain to the lumbar space. These processes are approximated here as a sequential series of compartments. More elaborate models that account for brain and subarachnoid space anatomy and flow may allow more accurate determination of the rates of Aβ peptide irreversible loss and production. Thus, the sensitivities reported here are those of the current compartmental model, not of the underlying biological system, which has yet to be fully elucidated. The current dataset is also not rich enough to identify the rates of production and irreversible loss of APP and C99. Additional kinetic data relevant to the production and irreversible loss of APP and C99 would certainly improve the estimation of Aβ production and irreversible loss rate constants. Also, measurement of concentrations of various Aβ peptides has a large impact on the estimates of the production rate constants for the Aβ peptides, and improvements in the precision and accuracy of concentration measurements would greatly aid future studies.

These data demonstrated that the FSR and monoexponential FCR previously used to characterize production and irreversible loss of Aβ peptides actually reflect the values of multiple parameters within a complicated system, and are not pure measures of production or irreversible loss. In steady-state studies, it is shown that estimation of the production rate is greatly enabled by combining isotope labeling data with concentration or pool sizes measurements. This also provides a mechanism for the observed decrease in CSF concentration of Aβ42 in Alzheimer's disease. The irreversible loss and exchange rate constants for Aβ peptides dominate the shape of the isotopic enrichment time course curve, and both constants may be readily determined by fitting the entire time course to the compartmental model. The later phases of the labeling process are better suited to resolve the irreversible loss and exchange processes of Aβ42. The conclusions of this study should enhance the design and interpretation of isotope-labeling experiments applied in the central nervous system.

### REFERENCES FOR EXAMPLES 11-16

[1] Hardy J, et al. “The amyloid hypothesis of Alzheimer's disease: progress and problems on the road to therapeutics.” Science, 2002, pp. 353-356, Vol. 297.
[2] Haass C, et al. “Take five—BACE and the [gamma]-secretase quartet conduct Alzheimer's amyloid [beta]-peptide generation.” EMBO J., 2004, pp. 483-488, Vol. 23.
[3] Bitan, G., et al. “Amyloid beta-protein (Abeta) assembly: Abeta 40 and Abeta 42 oligomerize through distinct pathways.” Proc. Natl. Acad. Sci. U.S.A., 2003, pp. 330-335, Vol. 100.
[4] Pellarin, R., et al. “Interpreting the aggregation kinetics of amyloid peptides.” J. Mol. Biol., 2006, pp. 882-892, Vol. 360.
[5] Bateman, R. J., et al. “Human amyloid-beta synthesis and clearance rates as measured in cerebrospinal fluid in vivo.” Nat. Med., 2006, pp. 856-861, Vol. 12.
[6] Wolfe, R. R., et al. “Isotope Tracers in Metabolic Research: Principles and Practice of Kinetic Analysis.” John Wiley & Sons, Hoboken, N.J. (2005)
[7] Mawuenyega, K. G., et al. “Decreased clearance of CNS beta-amyloid in Alzheimer's disease.” Science, 2010, p. 1774, Vol. 330.
[8] Elbert, D. L., et al. “Fractional synthesis and clearance rates for amyloid beta reply.” Nat. Med., 17 (2011), pp. 1179-1180.
[9] Potter, R., et al. “Increased in vivo Amyloid-β42 production, exchange, and irreversible loss in presenilin mutations carriers.” Sci. Transl. Med., 2013, pp. 189ra77, Vol. 5.
[10] Jankowsky, J. L., et al. “Mutant presenilins specifically elevate the levels of the 42 residue beta-amyloid peptide in vivo: evidence for augmentation of a 42-specific gamma secretase.” Hum. Mol. Genet., 2004, pp. 159-170, Vol. 13.
[11] Cobelli, C., et al. “Parameter and structural identifiability concepts and ambiguities: a critical review and analysis.” Am. J. Physiol., 1980, pp. R7-R24, Vol. 239.
[12] Bellman, R., et al. “On structural identifiability.” Math. Biosci., 1970, pp. 329-339, Vol. 7.
[13] Bateman, R. J., et al. “Clinical and biomarker changes in dominantly inherited Alzheimer's disease.” N. Engl. J. Med., 2012, pp. 795-804, Vol. 367.
[14] Edland, S. D., et al. “Fractional synthesis and clearance rates for amyloid beta.” Nat. Med., 17 (2011), pp. 1178-1179 (author reply 9-80).
[15] Ramakrishnan, R. “Studying apolipoprotein turnover with stable isotope tracers: correct analysis is by modeling enrichments.” J. Lipid Res., 2006, pp. 2738-2753, Vol. 47.
[16] Zilversmit, D. B. “The design and analysis of isotope experiments.” Am. J. Med., 1960, pp. 832-848, Vol. 29.
[17] Patterson, B. W. “Use of stable isotopically labeled tracers for studies of metabolic kinetics: an overview.” Metabolism, 1997, pp. 322-329, Vol. 46.
[18] Bateman, R. J., et al. “Fluctuations of CSF amyloid-beta levels: implications for a diagnostic and therapeutic biomarker.” Neurology, 2007, pp. 666-669, Vol. 68.
[19] Welander, H., et al. “Abeta43 is more frequent than Abeta40 in amyloid plaque cores from Alzheimer disease brains.” J. Neurochem., 2009, pp. 697-706, Vol. 110.
[20] Portelius, E., et al. “Distinct cerebrospinal fluid amyloid beta peptide signatures in sporadic and PSEN1 A431E-associated familial Alzheimer's disease.” Mol. Neurodegener., 2010, pp. 2, Vol. 5.
[21] Bateman, R. J., et al. “A gamma-secretase inhibitor decreases amyloid-beta production in the central nervous system.” Ann. Neurol., 2009, pp. 48-54, Vol. 66.
[22] Cook, J. J., et al. “Acute gamma-secretase inhibition of nonhuman primate CNS shifts amyloid precursor protein (APP) metabolism from amyloid-beta production to alternative APP fragments without amyloid-beta rebound.” J. Neurosci., 2010, pp. 6743-6750, Vol. 30.

Having described the invention in detail, it will be apparent that modifications and variations are possible without departing from the scope of the invention defined in the appended claims. Those of skill in the art should, however, in light of the present disclosure, appreciate that many changes could be made in the specific embodiments that are disclosed and still obtain a like or similar result without departing from the spirit and scope of the invention, therefore all matter set forth herein is to be interpreted as illustrative and not in a limiting sense.

While the present disclosure has been described with reference to various embodiments, it will be understood that these embodiments are illustrative and that the scope of the disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. An amyloid kinetics modeling system comprising at least one computing system further comprising at least one processor, at least one data storage device, a memory, and one or more hardware-implemented modules; wherein the at least one data storage device includes stored instructions which when executed by the processor cause the one or more hardware-implemented modules to generate a model for simulating a time course of enrichment kinetics of at least one Aβ isoform, the system comprising:
a plasma module to generate an infusion rate of a labeled moiety into the plasma of a patient determined by an infusion rate constant, and to simulate transport of the labeled moiety across the blood brain barrier (BBB) of the patient determined by one or more transport constants;
a brain tissue module to determine a rate of incorporation of the labeled moiety into APP and formation of C99 according to a degradation rate constant;
an amyloid kinetics module to determine a rate of cleavage of the C99 to form at least one Aβ isoform according to at least one isoform formation rate constant, and the amyloid kinetics module to simulate subsequent kinetics of the at least one Aβ isoform within the brain of the patient;
a CSF module to determine a rate of transport of the at least one Aβ isoform into the CSF of the patient
a model tuning module to iteratively adjust a set of model parameters defining a dynamic response of the model to input data regarding a measured time history of plasma leucine enrichment and wherein the model tuning module generates base enrichment data that is received at the plasma module to optimize predicted enrichment kinetics against measured enrichment kinetics of the at least one Aβ isoform in the patient; and
a GUI module to generate one or more forms used to receive inputs to the system and to generate one or more displays of data generated by the one or more hardware-implemented modules.

2. The system of claim 1, wherein the plasma module comprises plasma amino acid compartment to simulate a plasma concentration of at least one amino acid, wherein the plasma concentration of the at least one amino acid is determined using labeled amino acid input data comprising a measured time history of an infusion of a labeled amino acid into a patient.

3. The system of claim 2, wherein the brain tissue module further comprises:
a) an APP compartment to simulate a total amount of APP, and wherein the brain tissue module determines the rate of incorporation of the labeled moiety into APP using the labeled amino acid data received from the plasma module; and
b) a C99 compartment to simulate a total amount of C99 c-terminal fragments; wherein the brain tissue module determines a C99 formation rate comprising a rate of formation of the C99 c-terminal fragments simulated in the C99 compartment and determines a C99 clearance rate comprising a rate of disappearance of the C99 c-terminal fragments from the C99 compartment.

4. The system of claim 1, wherein the amyloid kinetics module comprises a soluble Aβ42 isoform compartment to simulate an amount of a soluble Aβ42 isoform and a recycled Aβ42 compartment to simulate a total amount of incorporated Aβ42 isoform; wherein the amyloid kinetics module:
determines an Aβ42 isoform formation rate comprising a rate of formation of soluble Aβ42 isoform from the C99 c-terminal fragments of the C99 compartment;
determines an Aβ42 isoform clearance rate comprising a rate of disappearance of Aβ42 isoforms from the soluble Aβ42 isoform compartment; and
determines an Aβ42 incorporation rate comprising a rate of transformation of the soluble Aβ42 isoform to the incorporated Aβ42 isoform.

5. The system of claim 4, wherein the amyloid kinetics module further comprises a soluble comparison Aβ isoform compartment to simulate an amount of a soluble comparison Aβ isoform; wherein the amyloid kinetics module:
determines a comparison Aβ isoform formation rate comprising a rate of formation of soluble comparison Aβ isoform from the C99 c-terminal fragments; and
determines a comparison Aβ isoform clearance rate comprising a rate of disappearance of soluble comparison Aβ isoforms from the soluble comparison Aβ isoform compartment.

6. The system of claim 1, wherein the CSF module comprises a CSF Aβ42 compartment to simulate a total amount of CSF Aβ42 isoforms; wherein the CSF module:
determines a CSF Aβ42 transfer rate comprising a rate of transfer of soluble Aβ42 isoform from the soluble Aβ42 compartment of the amyloid kinetics module to the CSF Aβ42 compartment; and
determines a CSF Aβ42 clearance rate comprising a rate of disappearance of CSF Aβ42 from the CSF Aβ42 pool.

7. The system of claim 6, wherein the comparison Aβ isoform is chosen from Aβ38 and Aβ40.

8. The system of claim 6, wherein the CSF module further comprises a CSF comparison Aβ isoform compartment to simulate a total amount of CSF comparison Aβ isoforms; wherein the CSF module determines:
a CSF comparison Aβ isoform transfer rate comprising a rate of transfer of soluble comparison Aβ isoform from the soluble comparison Aβ isoform compartment to the CSF comparison Aβ isoform compartment; and
determines a CSF comparison Aβ isoform clearance rate comprising a rate of disappearance of CSF comparison Aβ isoform from the CSF comparison Aβ isoform compartment.

9. The system of claim 8, wherein the comparison Aβ isoform is chosen from Aβ38 and Aβ40.

10. The system of claim 1 further comprising a blood enrichment module to determine transport of the at least one Aβ isoform into the blood of the patient.

11. A system for estimating the kinetics of amyloid-beta (Aβ) in the CNS of a patient, the system comprising at least one processor, at least one data storage device, a memory, and one or more hardware-implemented modules; wherein the at least one data storage device includes stored instructions which when executed by the processor cause the one or more hardware-implemented modules:
a) simulate a plasma amino acid compartment comprising a plasma concentration of at least one amino acid;
b) estimate an APP incorporation rate comprising a rate of incorporation of the at least one amino acid from the plasma amino acid compartment into an APP molecule in a simulated APP compartment;
c) estimate the APP compartment comprising a total amount of APP molecules;
d) estimate a C99 formation rate comprising a rate of formation of a C99 c-terminal fragment in a simulated C99 compartment from the APP molecules, the C99 compartment comprising a total amount of the C99 c-terminal fragments;
e) estimate a C99 clearance rate comprising a rate of disappearance of the C99 c-terminal fragment from the C99 compartment;
f) estimate at least one free Aβ isoform formation rate, each free Aβ isoform formation rate comprising a rate of formation of a free Aβ isoform in a simulated free Aβ compartment from the C99 c-terminal fragments, the free Aβ; compartment comprising the total amount of all free Aβ isoforms;
g) estimate at least one free Aβ isoform clearance rate, each free Aβ isoform clearance rate comprising a rate of disappearance of one of the free Aβ isoforms from the free Aβ compartment;
h) estimate at least one free Aβ incorporation rate, each free Aβ incorporation rate comprising a rate of transformation of a free Aβ isoform to an incorporated Aβ isoform in a simulated recycled Aβ compartment, and
i) estimate at least one Aβ recycling rate, each Aβ recycling rate comprising a rate of recycling an incorporated Aβ isoform in the recycled Aβ compartment back into a free Aβ isoform in the free Aβ compartment;
j) estimate at least one CSF Aβ transfer rate, each Aβ transfer rate comprising a rate of transfer of one free Aβ isoform from the free Aβ compartment to a simulated CSF Aβ compartment, the CSF Aβ compartment comprising the total amount of CSF Aβ isoforms; and
k) estimate at least one CSF Aβ clearance rate, each CSF Aβ clearance rate comprising a rate of disappearance of one CSF Aβ isoform from the CSF Aβ compartment.

12. The system of claim 11, wherein the Aβ isoforms are chosen from Aβ38, Aβ40, and Aβ42.

13. The system of claim 12, wherein:
at least a portion of the plasma amino acid compartment comprises a plasma concentration of at least one labeled amino acid;
at least a portion of the APP compartment comprises an amount of enriched APP molecules incorporating the at least one labeled amino acid;
at least a portion of the C99 compartment further comprises an amount of enriched C99 c-terminal fragments formed from the amount of enriched APP molecules; and
at least a portion of the Aβ isoforms further comprises an amount of enriched Aβ isoforms formed from the amount of enriched C99 c-terminal fragments.

14. The system of claim 11, wherein instructions executed by the processor cause the one or more hardware-implemented modules to estimate at least one CSF Aβ delay, each CSF Aβ delay comprising a delay in the transfer of one free Aβ isoform from the free Aβ compartment to the CSF Aβ compartment.

15. The system of claim 11, wherein the at least one CSF Aβ transfer rate is represented by a fluid flow of ISF within the brain.

16. A non-transitory compute readable medium including instructions for generating an amyloid kinetics modeling system and executing a simulation of the modeling system to estimate a time course of enrichment kinetics of at least one Aβ isoform, the instructions, executable by a processor, comprising:
generating an infusion rate of a labeled moiety into the plasma of a patient determined by an infusion rate constant,
simulating transport of the labeled moiety across the blood brain barrier (BBB) of the patient determined by one or more transport constants;
determining a rate of incorporation of the labeled moiety into APP and formation of C99 according to a degradation rate constant;
determining a rate of cleavage of the C99 to form at least one Aβ isoform according to at least one isoform formation rate constant;
simulating subsequent kinetics of the at least one Aβ isoform within the brain of the patient;
determining a rate of transport of the at least one Aβ isoform into the CSF of the patient;
iteratively adjusting a set of model parameters defining a dynamic response of the model to input data regarding a measured time history of plasma leucine enrichment;
generating base enrichment data that is used to optimize predicted enrichment kinetics against measured enrichment kinetics of the at least one Aβ isoform in the patient;
generating one or more forms used to receive inputs to the system; and
generating one or more displays of data.

17. The non-transitory compute readable medium of claim 16, wherein the instructions further comprise determining transport of the at least one Aβ isoform into the blood of the patient.

18. The non-transitory compute readable medium of claim 16, wherein the instructions further comprise simulating a plasma amino acid compartment to simulate a plasma concentration of at least one amino acid, wherein the plasma concentration of the at least one amino acid is determined using labeled amino acid input data comprising a measured time history of an infusion of a labeled amino acid into a patient.

19. The non-transitory compute readable medium of claim 2, wherein the instructions further comprise:
simulating a total amount of APP;
determining the rate of incorporation of the labeled moiety into APP using the labeled amino acid data;
simulating a total amount of C99 c-terminal fragments;
determining a C99 formation rate comprising a rate of formation of the C99 c-terminal fragments simulated in the C99 compartment; and
determining a C99 clearance rate comprising a rate of disappearance of the C99 c-terminal fragments from the C99 compartment.

20. The non-transitory compute readable medium of claim 1, wherein the instructions further comprise:
generating a soluble Aβ42 isoform compartment to simulate an amount of a soluble Aβ42 isoform;
generating a recycled Aβ42 compartment to simulate a total amount of incorporated Aβ42 isoform;
determining an Aβ42 isoform formation rate comprising a rate of formation of soluble Aβ42 isoform from the C99 c-terminal fragments of the C99 compartment;
determining an Aβ42 isoform clearance rate comprising a rate of disappearance of Aβ42 isoforms from the soluble Aβ42 isoform compartment; and
determining an Aβ42 incorporation rate comprising a rate of transformation of the soluble Aβ42 isoform to the incorporated Aβ42 isoform.

21. The non-transitory compute readable medium of claim 4, wherein the instructions further comprise:
generating a soluble comparison Aβ isoform compartment to simulate an amount of a soluble comparison Aβ isoform;
determining a comparison Aβ isoform formation rate comprising a rate of formation of soluble comparison Aβ isoform from the C99 c-terminal fragments; and
determining a comparison Aβ isoform clearance rate comprising a rate of disappearance of soluble comparison Aβ isoforms from the soluble comparison Aβ isoform compartment.

22. The non-transitory compute readable medium of claim 1, wherein the instructions further comprise:
generating a CSF Aβ42 compartment to simulate a total amount of CSF Aβ42 isoforms;
determining a CSF Aβ42 transfer rate comprising a rate of transfer of soluble Aβ42 isoform from the soluble Aβ42 compartment to the CSF Aβ42 compartment; and
determining a CSF Aβ42 clearance rate comprising a rate of disappearance of CSF Aβ42 from the CSF Aβ42 pool.

23. The system of non-transitory compute readable medium 22, wherein the comparison Aβ isoform is chosen from Aβ38 and Aβ40.

24. The non-transitory compute readable medium of claim 22, wherein the instructions further comprise:
generating a CSF comparison Aβ isoform compartment to simulate a total amount of CSF comparison Aβ isoforms;
determining a CSF comparison Aβ isoform transfer rate comprising a rate of transfer of soluble comparison Aβ isoform from the soluble comparison Aβ isoform compartment to the CSF comparison Aβ isoform compartment; and
determines a CSF comparison Aβ isoform clearance rate comprising a rate of disappearance of CSF comparison Aβ isoform from the CSF comparison Aβ isoform compartment.

25. The non-transitory compute readable medium of claim 24, wherein the comparison Aβ isoform is chosen from Aβ38 and Aβ40.


SEQUENCE LISTING (1 sequence)

SEQ ID NO: 1 - protein, 42 aa, Homo sapiens [sequence omitted]